(19) United States
(12) Patent Application Publication
(10) Pub. No.: US 2023/0384333 A1
(43) Pub. Date: Nov. 30, 2023

(54) GPCR SCREENING METHOD TO IDENTIFY NON-HALLUCINOGENIC COMPOUNDS

(71) Applicant: The Regents of the University of California, Oakland, CA (US)

(72) Inventors: Chunyang DONG, Oakland, CA (US); Calvin LY, Oakland, CA (US); David OLSON, Oakland, CA (US); Lin TIAN, Oakland, CA (US)

(21) Appl. No.: 18/248,934

(22) PCT Filed: Oct. 13, 2021
(86) PCT No.: PCT/US2021/054669

§ 371 Date: Apr. 13, 2023

Related U.S. Application Data
(60) Provisional application No. 63/091,041, filed on Oct. 13, 2020.
(60) Provisional application No. 63/182,669, filed on Apr. 30, 2021.

(51) Int. Cl. G01N 33/94; G01N 33/58; C07K 14/72

(52) U.S. Cl. CPC G01N 33/9466; G01N 33/582; C07K 14/723; G01N 2333/726

(57) ABSTRACT

New fluorescent biosensors are provided for use in methods of detecting a ligand-induced hallucinogenic conformational change of a G Protein-Coupled Receptor (GPCR), detecting a hallucinogenic compound, detecting a non-hallucinogenic antidepressant compound, measuring the hallucinogenic potential of a compound, measuring the antipsychotic potential of a compound, and suitable kits thereof.

### CROSS-REFERENCES TO RELATED APPLICATIONS

This application claims priority to U.S. Provisional Application No. 63/091,041, filed Oct. 13, 2020, and 63/182,669, filed Apr. 30, 2021, each of which is incorporated herein in its entirety for all purposes.

### STATEMENT AS TO RIGHTS TO INVENTIONS MADE UNDER FEDERALLY SPONSORED RESEARCH AND DEVELOPMENT

This invention was made with Government support under Grant Nos. R01GM128997 and U01NS013522 awarded by the National Institutes of Health. The Government has certain rights in this invention.

### BACKGROUND OF THE INVENTION

G protein coupled receptors (GPCRs) sense small differences in the molecular structures of ligands and translate these into protein conformational ensembles with distinct functional consequences relevant to drug discovery. The serotonin 2A receptor (5-HT2AR) is an excellent example, being the target of atypical antipsychotics, classic hallucinogens, and neural plasticity-promoting psychoplastogens. Tools capable of directly assessing 5-HT2AR conformations are currently lacking. Here, we report the development of psychLight—a genetically encoded fluorescent biosensor capable of reporting ligand-induced hallucinogenic conformations of the 5-HT2AR. Using a stable cell line expressing psychLight, we were able to predict the hallucinogenic potential of several novel compounds and identify a non-hallucinogenic, psychedelic-inspired small molecule with neural plasticity-promoting and long-lasting antidepressant properties. Additionally, psychLight permits imaging of cortical and subcortical serotonin dynamics in freely behaving mice with millisecond resolution. The hallucinogen sensor described here will enable the rapid identification of designer drugs of abuse and facilitate the development of safer, next-generation neurotherapeutics.

Approximately 35% of all FDA-approved medications target GPCRs, as these receptors are implicated in a variety of diseases and can be readily controlled with small molecules. Due to their abilities to access a myriad of conformational states, GPCRs can activate numerous canonical and non-canonical signaling pathways through G proteins, arrestins, or other effectors depending on the specific conformational ensemble stabilized by the ligand. Therefore, two ligands binding to the same GPCR can elicit entirely different functional effects—a phenomenon known as functional selectivity, or biased agonism. Recent drug discovery efforts have attempted to exploit differences in functional selectivity to identify pharmaceuticals with fewer side effects.

Current technologies for assessing functional selectivity rely on measuring the recruitment of specific proteins (e.g., β-arrestin) or the accumulation of downstream signaling molecules (e.g., cAMP). Few, if any, directly probe the conformational states of the GPCR responsible for producing specific effects. Moreover, there is a dearth of methods for measuring non-canonical functional selectivity in a high-throughput manner, and none of the above-mentioned methods are amenable to in vivo use.

Recently, we reported a modular strategy for creating genetically encoded fluorescent sensors based on the structures of GPCRs. Here, we applied this strategy to develop a fluorescent biosensor capable of detecting hallucinogenic conformations of the serotonin 2A receptor (5-HT2AR). Ligands of the 5-HT2AR were some of the first small molecules to demonstrate biased agonism. Since that initial report, 5-HT2A ligands have been shown to couple to a variety of signal transduction pathways via 5-HT2A monomers and heterodimers leading to distinct transcriptome profiles and behavioral effects. Furthermore, 5-HT2AR ligands represent some of the most important drugs in neuropsychiatry including atypical antipsychotics like clozapine, hallucinogens such as lysergic acid diethylamide (LSD), and neural plasticity-promoting compounds known as psychoplastogens.

Psychedelics are a class of psychoplastogens being reinvestigated as potential medicines due to their long history of demonstrating clinical efficacy for treating diseases such as depression, post-traumatic stress disorder (PTSD), and substance use disorder (SUD). The 5-HT2AR has been shown to mediate both the hallucinogenic and psychoplastogenic effects of psychedelics; however, it is currently unclear if both are necessary for the therapeutic effects of these drugs. Recent rodent studies using subhallucinogenic doses and non-hallucinogenic congeners suggest that the hallucinogenic effects of 5-HT2A ligands might not be necessary to produce positive behavioral outcomes. Moreover, we recently reported that the hallucinogenic and psychoplastogenic effects of 5-HT2A ligands can be decoupled through careful chemical design. Given the importance of 5-HT2A ligands in medicine and the critical need to develop a cellular assay for hallucinogenic potential, we used the structure of the 5-HT2AR to engineer a fluorescence-based hallucinogen sensor known as psychLight.

Using psychLight we were able to identify novel hallucinogenic drugs of abuse as well as a non-hallucinogenic compound with neural plasticity-promoting and antidepressant properties similar to hallucinogenic psychedelics. Finally, as psychLight displays high sensitivity to serotonin (5-HT), we were able to use it to probe brain region-specific 5-HT release in freely behaving mice during fear conditioning. Our results not only demonstrate that psychLight can be used for the in vivo detection of 5-HT with high spatial and temporal precision, they also have important implications for the identification of designer hallucinogens as well as safe and effective next-generation medicines for treating neuropsychiatric diseases.

### BRIEF SUMMARY OF THE INVENTION

In one embodiment, the present invention provides a method of detecting a ligand-induced hallucinogenic conformational change of a G Protein-Coupled Receptor (GPCR), the method comprising: contacting the ligand with a fluorescent biosensor under conditions for the ligand to bind to the fluorescent biosensor, wherein the fluorescent biosensor comprises the GPCR, and a circularly permuted green fluorescent protein (cpGFP) integrated into the third intracellular loop (IL3) of the GPCR; and measuring the change in fluorescence of the biosensor, thereby detecting the conformational change.

In another embodiment, the present invention provides a method of detecting a hallucinogenic compound, the method comprising: contacting a compound with a fluorescent biosensor under conditions for the compound to bind to the fluorescent biosensor, wherein the fluorescent biosensor comprises a G Protein-Coupled Receptor (GPCR), and a circularly permuted green fluorescent protein (cpGFP) integrated into the third intracellular loop (IL3) of the GPCR; and measuring the change in fluorescence of the biosensor, wherein an increase in fluorescence indicates the presence of the hallucinogenic compound, thereby detecting the hallucinogenic compound.

In another embodiment, the present invention provides a method of detecting a non-hallucinogenic antidepressant compound, the method comprising: contacting a compound with a fluorescent biosensor under conditions for the compound to bind to the fluorescent biosensor, wherein the fluorescent biosensor comprises a G Protein-Coupled Receptor (GPCR), and a circularly permuted green fluorescent protein (cpGFP) integrated into the third intracellular loop (IL3) of the GPCR; and measuring the change in fluorescence of the biosensor, wherein a decrease in fluorescence indicates the presence of the non-hallucinogenic antidepressant compound, thereby detecting the non-hallucinogenic antidepressant compound.

In another embodiment, the present invention provides a fluorescent biosensor comprising: a 5-HT2A receptor; and a circularly permuted green fluorescent protein (cpGFP) integrated in the third intracellular loop (IL3) of the 5-HT2A receptor.

In another embodiment, the present invention provides a fluorescent biosensor comprising: a 5-HT2A receptor; a circularly permuted green fluorescent protein (cpGFP) integrated in the third intracellular loop (IL3) of the 5-HT2A receptor; and an ER export peptide on the C-terminus.

In another embodiment, the present invention provides a method of measuring the hallucinogenic potential of a compound, comprising contacting the compound with a fluorescent biosensor of the present invention, and measuring the agonist effect of the compound on the fluorescent biosensor.

In another embodiment, the present invention provides a method of measuring the antipsychotic potential of a compound, comprising contacting the compound with a fluorescent biosensor of the present invention, and measuring the agonist or antagonist effect of the compound on the fluorescent biosensor.

In another embodiment, the present invention provides a method of identifying a hallucinogenic compound from a non-hallucinogenic compound, the method comprising: contacting a compound with a fluorescent biosensor under agonist conditions and measuring a first fluorescence signal of the compound, wherein an increase in the first fluoresence signal compared to a first control indicates the compound is hallucinogenic; contacting the compound with the fluorescent biosensor under antagonist conditions and measuring a second fluoresence signal of the compound, wherein a decreased second fluoresence signal compared to a second control indicates the compound is non-hallucinogenic; and combining the first fluoresence signal and the second fluorescence signal to calculate a ligand score where a positive ligand score identifies the compound as a hallucinogenic compound and a negative ligand score identifies the compound as a non-hallucinogenic compound.

In another embodiment, the present invention provides a kit comprising a fluorescent biosensor of the present invention.

In another embodiment, the present invention provides a cell comprising a fluorescent biosensor of the present invention.

### BRIEF DESCRIPTION OF THE DRAWINGS

FIG. 1. Development and Characterization of PsychLight. (A) Modeled structure of psychLight1 consisting of the human 5-HT2AR linked to a cpGFP. Blue and green represent the N-terminals of the 5-HT2AR and the cpGFP, respectively, while red and yellow represent the C-terminals. (B) Sequence alignment of the B2AR and D1R with the 5-HT2AR. A fluorescent module connected to the receptor via two linking regions replaced IL3. The LSS and DQL linker regions were taken from the structure of GCaMP. Two variable amino acids (XX) flanked the cpGFP. (C) Concentration response studies using psychLight1 expressed in HEK293T cells revealed that agonists (5-HT), but not antagonist/inverse agonists (KETSN, MDL) activate the sensor. (D) Concentration-response studies reveal that hallucinogens, but not non-hallucinogenic congeners, activate psychLight. Hallucinogen/non-hallucinogen pairs representing the ergoline, tryptamine, and amphetamine families of psychedelics are shown. (E) Non-hallucinogenic 5-HT2AR ligands can reduce the signal generated by 5-HT in a concentration-dependent manner. Thus, they bind to the sensor, but exhibit no efficacy. (F) Hallucinogenic potencies in humans correlate well with psychLight potencies but not with Emax values. (G) A HEK293T cell line stably expressing psychLight2 (PSYLI2) was created using adeno-associated virus. The assay is suitable for use in a 96-well plate format. The sensor exhibits constitutive activity (light green), and thus, can be used to detect hallucinogenic agonists (dark green) and non-hallucinogenic inverse agonists (white). (H) Small changes to the structure of 5-HT can impact the Emax values obtained using PSYLI2 cells.

FIG. 2. PsychLight Enables the Rapid Determination of Hallucinogenic Potential. (A) Dot plot indicating ΔF/F values for the assay run in agonist mode. Dotted lines indicate 1 STD from the mean of the VEH (DMSO) control. Values great than 1 STD are likely to be hallucinogenic 5-HT2AR ligands. Colors correspond to specific compounds in B. (B-D) Data from the psychLight assay run in agonist (B) and antagonist mode (C) were used to calculate a ligand score (D). Ligand scores that are black indicate that a compound is unlikely to be a ligand for the 5-HT2AR (black set to −4.2, the value for VEH control). Ligand scores that are red and blue indicate hallucinogenic and non-hallucinogenic 5-HT2AR ligands, respectively. Compounds with ligand scores exceeding the values for LSD (20.9) and MDL100907 (−57.9), are shown as the brightest red and blue colors, respectively.

FIG. 3. PsychLight Predicts the Hallucinogenic Properties of 5-Halo-DMT Derivatives. (A) Structures of 5-halo-DMT derivatives. The sizes of the circles correspond to the relative Van der Waals radii of the halogens. (B) PsychLight predicts that 5-F-DMT and 5-Cl-DMT, but not 5-Br-DMT, to be hallucinogenic. (C) Schematic showing that hallucinogenic 5-HT2A ligands produce head-twitch response (HTR) behavior, making this test an excellent in vivo assay for hallucinogenic potential. (D) HTR studies confirm hallucinogenic properties of 5-halo-DMT derivatives predicted by psychLight. (E) Activation of psychLight correlates with HTR, but not with effects on locomotion.

FIG. 4. AAZ-A-154 is a Non-Hallucinogenic 5-HT2AR Ligand with Antidepressant Properties. (A) Structural similarity between AAZ-A-154 and the hallucinogenic psychoplastogen 5-MeO-DMT. (B) AAZ-A-154 does not produce a HTR at any dose. (C) A high dose of AAZ-A-154 (100 mg/kg) reduces locomotion. (D) Representative images demonstrating that AAZ-A-154 promotes dendritic branching. (E-F) Nmax values (F) of the Sholl plots (E) indicate that AAZ-A-154 increases dendritic arbor complexity. (G) The effects of AAZ-A-154 on dendritic growth is blocked by the 5-HT2AR antagonist ketanserin (KETSN). (H) AAZ-A-154 (20 mg/kg) produces fast (30 min) and long-lasting (1 week) antidepressant-like effects in the FST comparable to ketamine. (I) Neither WT nor VMAT2-HET mice exhibited a preference for either of two bottles containing water (W-W). When given the choice between water and a 1% sucrose solution (W-S), only WT mice displayed a sucrose preference. The day after sucrose preference was initially assessed, both groups were given a single injection of AAZ-A-154 (15 mg/kg). Sucrose preference was assessed immediately following compound administration and again at various points over the course of 28 days. AAZ-A-154 eliminates anhedonia in VMAT2-HET mice for up to 16 days. Total fluid consumption was not different between genotypes at a̅n̅y̅ t̅i̅m̅e̅ p̅o̅i̅n̅t̅.̅ N̅=̅ 11 mic̅e̅/̅g̅e̅n̅o̅t̅y̅p̅e̅.̅ V̅E̅H̅=̅vehicle, KET=ketamine, KETSN=ketanserin, AAZ=AAZ-A-154.

FIG. 5. PsychLight Can Measure 5-HT Dynamics In Vivo. (A) 2P time-lapse images of a dendrite expressing psychLight2. (B) ΔG/R before and 5 min after 5-HT bath application (7 ROIs, 4 cells). (C) GFP signals imaged by 2P line scanning following 2P 5-HT uncaging (1 pulse of 10 ms duration). (D) Averaged traces and summary ΔG/R from stimulated (filled bar; 76 ROIs, 11 cells) and mock-stimulated (open bar; 32 ROIs, 6 cells) neurons. (E) Schematic for fiber photometry experiments performed in the DRN, BNST, BLA, and OFC following viral expression of psychLight. A total of 15 tone/shock pairings were presented before and after administration of escitalopram (ESC, 10 mg/kg). (F-I) Fiber photometry measuring 5-HT dynamics in response to auditory fear conditioning in the DRN (F), the BNST (G), the BLA (H), and the OFC (I). Mice were injected with either AAV9.hSynapsin-psychLight2 (G-I) or AAV8.hSynapsin-psychLight2 (F), followed by optical fiber implantation into the corresponding brain regions. Gray and pink boxes indicate tones and foot shocks, respectively. Black and green trace indicate conditions without (−ESC) and with (+ESC) escitalopram, respectively. Shaded area represents SEM.

FIG. 6. Engineering a Sensor for Hallucinogenic Conformations of the 5-HT2A Receptor. (A) 5-HT2AR insertion sites for a fluorescent module containing a cpGFP were screened. The top-performing variant advanced to the next stage. (B) After an insertion site had been selected, the residues flanking the cpGFP were varied. The top-performing variant advanced to the next stage. (C) Point mutations in the cpGFP were made. The top-performing variant was named psychLight1. The top-performing variants are circled in red.

FIG. 7. Comparison between PsychLight1 and PsychLight2. (A) Representative images of HEK293T cells or embryonic rat hippocampal neurons expressing either psychLight1 or psychLight2. scale bars=20 μm. (B) Representative line scans across the soma (HEK293T cells) or dendrites (neurons). The edges of the cells are highlighted in gray. Both psychLight1 and psychLight2 are expressed on the cell membranes of HEK293T cells. However, neuronal expression of psychLight1 is primarily intracellular. PsychLight2 is expressed on the surface of neurons to a greater extent. (C) PsychLight1 and psychLight2 respond similarly to positive controls (i.e., 5-HT or 5-MeO-DMT at 10 μM) and negative controls (i.e., 6-MeO-DMT or ketamine at 10 μM). The fluorescence intensities of psychLight1 and psychLight2 were measured using a confocal microscope and a high content imager, respectively. 5-HT=serotonin, 5-MeO=5-MeO-DMT, 6-MeO=6-MeO-DMT, KET=ketamine.

FIG. 8. LSD and LIS Exhibit Differences in Functional Selectivity at GPCR-Based Sensors. (A) Both LSD and LIS activate dLight as demonstrated by a comparison between their Eₘₐₓ values. However, only the hallucinogenic compound (i.e., LSD) can activate psychLight2. (B) The percent change in Eₘ. values for the activation of dLight vs psychLight are presented. The psychLight signal produced by LIS is reduced by 99.4% relative to dLight, while the LSD signal is only reduced by 69.3%. LSD=lysergic acid diethylamide; LIS=lisuride.

FIG. 9. Chemical Structures of the Compounds Synthesized in House. FIG. 9 shows he syntheses and characterization data for any novel compounds are shown in the supporting information.

FIG. 10. PsychLight is Only Activated by 5-HT. PsychLight fluorescence is not affected by glutamate, GABA, DA, or NE. The structures of these neurotransmitters and neuromodulators are shown.

FIG. 11. Development of a fluorescent sensor based on the 5-HT2A receptor. (A) Simulated structure of psychLight consisting of 5-HT2AR (gray), a linker (magenta) and a cpGFP (green). (B) Representative images of cultured dissociated hippocampal neurons transiently expressing psychLight1 and psychLight2. Scale bar, 20 ₘm. (C) PsychLight1-expressing HEK293T cells respond to ligands in a concentration-dependent manner. (D) PsychLight1 is activated by hallucinogenic 5-HT2A ligands, but not non-hallucinogenic compounds when treated at 10 ₘM. ****p<0.0001, **p<0.01 and *p<0.05, one-way ANOVA compared to KETSN with Dunnett's test. (E and F) Two-photon imaging of cultured cortical slices expressing psychLight2 (pL2) following bath application of 5-HT. (E) Representative images of a dendrite expressing psychLight2 (pL2) and tdTomato (tdT) before and after bath application of 50 mM 5-HT (imaged at 920 nm). (F) Fluorescence intensity changes in pL2 were normalized to the tdT signal, (_{D}pL2/tdT=111.1%±1.8%, n=7 region of interests [ROIs] from 4 cells; ****p<0.0001, unpaired t test). Scale bar, 1 mm. (G and H) Two-photon 5-HT uncaging evoked psychLight responses. (G) Representative apical dendrites imaged during two-photon uncaging of serotonin. Representative single-trial traces of fluorescent intensity changes (DF/F %) of pL2 and tdT are shown in response to single pulse uncaging (10 ms). Averaged traces of _{D}pL2/tdT in response to uncaging of Rubi-5HT (bottom) and without were shown. Scale bars, 1 ₘm. (H) Characterization of peak response of green to red ratio (_{D}pL2/tdT) normalized to the baseline for pL2 in response to single-pulse uncaging with and without RuBi-5-HT (_{D}pL2/tdT=111.2%±0.7%, n=76 ROIs from 11 cells (Rubi-5-HT); 101.3%±1.0% n=32 ROIs from 6 cells [mock 2P]), ****p<0.0001, unpaired t test. (I-K) Two-photon imaging of endogenous 5-HT release triggered by electrical stimuli in acute slices. (I) A representative two-photon image of BNST acute slice expressing psychLight2. Scale bar, 50 ₘm. (J) Single-trial response of psychLight2 to electrical stimuli (0.5 s, 4 V, 40 Hz, 20 pulses). (K) The averaged off-kinetics of two groups of ROIs exhibiting fast and slow off rates. (40 pulses: Tau_{fast}=0.997±0.0376 s, n=5 trials; Tau_{slow}=3.998±0.6103 s, n=6 trails), **p<0.01, unpaired t test. (L) Averaged-trial traces of psychLight2 in response to electrical stimuli in the presence of escitalopram (ESC, 50 ₘM), granisetron (GRN, 10 ₘM), and tetrodotoxin (TTX, 1 ₘM). Shaded area represents SEM. (M) Peak fluorescence changes in the absence (aCSF, n=11 trials from 3 mice) and presence of compounds (ESC, n=2 trials from 3 mice, ***p=0.0002; GRN, n=8 trials from 3 mice, ****p<0.0001; TTX n=9 trials from 3 mice, 40 pulses, one-way ANOVA compared to aCSF with Sidak's test). Data are represented by mean±SEM.

FIG. 12. PsychLight enables the detection of endogenous serotonin dynamics during fear conditioning using fiber photometry. (A) Expression of psychLight2 in the DRN, BNST, BLA and OFC near the location of fiber implantation. Scale bars, 500 mm. (B) Schematic illustrating the design of auditory fear conditioning experiments (30-s tone co-terminating with a 1.5-s foot shock, n=15 presentations). (C-F) Single-trial heatmap and averaged-trial traces of serotonin dynamics in DRN (C, n=135 trials from 9 animals), BNST (D, n=120 trials from 8 animals), BLA (E, n=90 trials from 6 animals), and OFC (F, n=90 trials from 6 animals) in response to a tone (blue) and foot shock (pink). ROC plots indicate true detection rate (TDR) against false-positive rate (FPR), and d⁰ is calculated by avg(Z score_{shock})/std(Z score_{baseline}). Average traces indicated by solid lines; shaded area represents SEM. DRN, dorsal raphe nucleus; BNST, bed nucleus of the stria terminalis; BLA, basolateral amygdala; OFC, orbitofrontal cortex.

FIG. 13. PsychLight is activated by hallucinogenic drugs in vivo and in vitro (A-C) PsychLight2 in vivo responses to drugs as measured by fiber photometry. (A) Expression of psychLight2 in the prelimbic cortex near the site of fiber implantation. Scale bar, 500 mm. (B) Averaged-trial traces of psychLight2 responses shown as Z score following injection of 50 mg/kg 5-MeO (magenta, i.p.). The number of head-twitch responses (bars) were also recorded and binned into 1-min intervals (n=3 animals). (C) Averaged-trial traces of psychLight2 responses following the injection either of the saline (VEH; top black) or an antagonist (4 mg/kg KETSN, bottom blue, i.p.) (n=3 animals). Average traces indicated by solid lines; shaded area represents SEM. (D-G) Concentration-response studies using HEK293T cells transiently expressing psychLight1. Hallucinogens of the ergoline, tryptamine, and amphetamine classes of psychedelics (magenta) were tested along with their non-hallucinogenic congeners (blue). Hallucinogens activated psychLight1 while their non-hallucinogenic congeners did not. n=3 cells from 3 different cell passages; Error bars represent SEM, *p<0.05, **p<0.01, ***p<0.001, ****p<0.0001, compared to the non-psychedelic drug, two-way ANOVA. (H) PsychLight1 _{EC50} values, but not _{Emax} values, correlate with hallucinogen potencies in humans. (I) PsychLight1 _{Emax} values differentiate hallucinogens and non-hallucinogens, but other measures of 5-HT2AR activation (e.g., phosphoinositide [PI]hydrolysis, Ca²⁺ mobilization, [³⁵S]GTPyS binding) do not. Data represented by the heatmap with a double color gradient from values above 0 (magenta to black) and data below 0 (black to blue). Data are normalized to 5-HT values within each experiment. Data for PI hydrolysis, Ca²⁺ mobilization, and [³⁵S]GTPyS binding were obtained from previous reports. PI hydrolysis data for 6-F-DET were estimated based on graphical data presented in Rabin et al. (2002). N/A indicates that the data are not available.

FIG. 14. Development of a medium-throughput psychLight-based pharmacological assay. (A) A lentivirus expressing psychLight2 under the EF1a promoter was used to engineer a HEK293T cell line stably expressing psychLight2 (PSYLI2). (B and C) Structure-function studies using a variety of structurally related tryptamines. (B) Structures of compounds. (C) PSYLI2 fluorescence in response to compound treatments (10 mM). Data are represented by mean±SEM, ****p<0.0001, **p<0.01 and *p<0.05, one-way ANOVA multiple comparison with Tukey's test. (D) A series of hallucinogenic and non-hallucinogenic compounds with known 5-HT2AR affinities were tested in agonist (abscissa) and antagonist (ordinate) modes. Dotted lines represent 1 SD from the VEH control (white). Hallucinogenic and non-hallucinogenic 5-HT2AR ligands are shown in red and blue, respectively. Compounds with weak affinity for the 5-HT2AR (*1-10 mM) are shown in gray, whereas compounds that are known to not bind to the 5-HT2AR are shown in black. Dots indicate averaged DF/F values (n=3 replicates from 3 passages of cells). (E) Heatmap of ligand scores. Ligand scores greater than 0 indicate compounds more likely to be hallucinogenic while scores less than 0 indicate compounds that are more likely to be non-hallucinogenic ligands of the 5-HT2AR.

FIG. 15. PsychLight accurately predicts hallucinogenic potentials of previously un-tested compounds. (A) Structures of 5-halo-DMT derivatives and AAZ-A-154. Colored circles indicate the relative size of the halogen atom compared to each other. (B) Both 5-F-DMT and 5-Cl-DMT produce positive ligand scores and induce head-twitches in mice. In contrast, 5-Br-DMT produces a negative ligand score and does not induce a HTR (n=4 mice). (C) All 5-halo-DMTs produce dose-dependent decreases in locomotion (n=4 mice). (D) Schild regression analysis reveals that AAZ-A-154 is a psychLight competitive antagonist (n=3 replicates from 1 passage of cells). (E) AAZ-A-154 does not trigger a HTR at any dose compared to that triggered by 5-MeO-DMT (n=4 mice). (F) AAZ-A-154 only decreases locomotion at a very high dose (100 mg/kg) (n=4 mice). Data are represented as mean±SEM. ****p<0.0001, ***p<0.001, and *p<0.05, versus the vehicle control, one-way ANOVA with Dunnett's test.

FIG. 16. A predicted non-hallucinogenic compound with antidepressant potential. (A) Representative images demonstrating that AAZ-A-154 promotes dendritic branching. Scale bar, 20 mm. (B) Maximal number of crossings _{(Nmax)} from Sholl plots (n=51-60 neurons). ****p<0.0001, ***p<0.001, one-way ANOVA with Dunnett's test. (C) The effects of AAZ (100 nM) on dendritic growth can be blocked by the 5-HT2R antagonist ketanserin (KETSN, 1 mM, n=39-58 neurons). ****p<0.0001, one-way ANOVA with Dunnett's test. (D) Schematic depicting the forced swim test design. AAZ-A-154 (20 mg/kg) produces fast (30 min) and long-lasting (1 week) antidepressant-like effects in the FST comparable to ketamine (KET) (n=12). ****p<0.0001, ***p<0.001, and *p<0.05, one-way ANOVA with Dunnett's test. Sucrose preference test reveals that AAZ (15 mg/kg) reduces anhedonia in VMAT2-HET mice for at least 12 days. W-W, water-water pairing; W-S, water-sucrose (1%) pairing. When given the choice between water and a 1% sucrose solution (W-S), only WT mice displayed a sucrose preference. Total fluid consumption was not different between genotypes at any time point. n=11 mice/genotype; data are represented as means and SEMs, **p<0.01 and *p<0.05, WT versus VMAT2-HET, repeated-measures ANOVA with Bonferroni corrected pairwise comparisons.

FIG. 17. Engineering a sensor for hallucinogenic conformations of the 5-HT2A receptor. A. Sequence alignment of b2A, DRD1, and 5-HT2A receptors. Initial insertion site of the LSSLI-cpGFP-NHDQL module was between K263 and S316 of the 5-HT2AR. The original cpGFP flanking residues (i.e., LI-cpGFP-NH) were chosen based on the sequence of dLight1.3. B. After the initial insertion site was determined, a total of 781 variants were screened to optimize psychLight. We screened 766 variants related to the aa composition of the linkers. The top performer (i.e., GY-MH, blue) advanced to the next stage of screening. Next, we performed insertion site optimization of the fluorescent module. A point mutation in TM5 (i.e., E264Q) and removal of 1 aa from TM6 yielded a better variant (red). (TM5-2aa, TM6-1aa=−4.2±0.8%. TM5-1aa, TM6-1aa=−0.5±0.8%. TM5+4aa, TM6-1aa=10.1±2.2%. Parent (no mutation)=22.1±3.9%. TM5+1aa, TM6-1aa=37.2±3.2%. TM6-1aa=40.8±1.7%. TM5 E264Q, TM6 N317K=44.2±1.8%. TM5 E264Q, TM6-1aa=53.2±0.9%. n=4 replicates from 4 passages of cells). Next, we made point mutations in intracellular loop 2. The top-performing variant was named psychLight1 (magenta) (P180G/I181G=33.7±6.8%. P180A/I181A=34.8±2.5%. Parent (no mutation)=49.4±5.1%. Q178A/I181A=49.4±5.1%. I181G=68.5±1.3%. P180A/I181G=72.9±5.7%. I181A=85.2±3.9%). n=3 replicates from 3 passages of cells. Data are represented as mean±SEM. ****p<0.0001, ***p<0.001, **p<0.01 and *p<0.05, one-way ANOVA compares to parent variant within the group with Dunnett's test. C-D. Optimization of psychLight membrane localization. Representative images of HEK293T cells expressing either psychLight1 or psychLight2. Scale bar: 20 mm. Both psychLight1 and psychLight2 are expressed on the cell membranes of HEK293T cells. However, neuronal expression of psychLight1 is primarily intracellular PsychLight2 is expressed on the surface of neurons to a greater extent. AFU=Arbitrary Fluorescence Units.

FIG. 18. Control experiments of psychLight recordings in HEK cells ex-vivo and in vivo. A-B. Application of 5-HT and KETSN to HEK293T cells in different orders reveals blocking effect of KETSN. A. The order of addition is 10 μM 5-HT followed by 10 μM KETSN and then 10 μM 5-HT. B. The order of addition is 10 μM KETSN followed by 10 μM 5-HT. C. Field stimulation of BNST brain slices followed by two photon imaging of psychLight with either aCSF (green) or a solution of 5-HT2A antagonist KETSN (10 μM, black). The effect of field stimulation was blocked by KETSN. N=5 slices. D. Quantification of the peak response in C (aCSF: 3.71±0.57%, KETSN: 0±0.36%, ***p<0.001, unpaired t test). E. Fiber photometry recordings of psychLight0-a control sensor that cannot bind to 5-HT due to a key point mutation (D155A). F. Side by side comparison of psychLight2 and psychLight0 (ctrl) fluorescence during foot shock, indicating psychLight2 is primarily detecting changes in 5-HT concentrations and not simply motion artifacts. DR-psychLight: −6.069±1.165%, DR−ctrl: −1.588±0.5303%, BLA−psychLight: −15.63±1.581%, BLA-ctrl: −1.253±0.7031%, BNST-psychLIght: −16.44±1.034%, BNST ctrl: −2.408±1.033%, OFC-psychLight: −13.79±2.835%, OFC-ctrl: 0.5163±0.3395%, *p<0.05, **p<0.01, ***p<0.001, ****p<0.0001, unpaired t test compared between each brain region. 5-HT=5-hydroxytryptamine; KETSN=ketanserin; ctrl=psychLight0; DRN=dorsal raphe nucleus; BNST=bed nucleus of the stria terminalis; BLA=basolateral amygdala; OFC=orbitofrontal cortex.

FIG. 19. Confocal imaging of psychLight1-expressing HEK293T cells after compound treatments and PSYLI2 characterization. A-B. The non-hallucinogenic ligands lisuride (A) and 6-MeO-DMT (B) can compete off 5-HT resulting in a reduction in psychLight fluorescence. C. PsychLight1 and psychLight2 respond similarly to positive controls (i.e., 5-HT or 5-MeO-DMT at 10 mM) and negative controls (i.e., 6-MeO-DMT or ketanserin at 10 mM). The fluorescence intensities of psychLight1 and psychLight2 were measured using a confocal microscope and a high content imager, respectively (Confocal with psychLight1: psychLight15-HT=77.3±2.4%, n=3; psychLight15-MeO=48.4±3.1%, n=3; psychLight16-MeO=−1.1±4.6%, n=3; psychLight1KETSN=−1.7±0.3%, n=3. High content imager with psychLight2: psychLight25-HT=38.2±2.4%, n=8; psychLight25-MeO=24.1±4.6% n=6; psychLight26-MeO=−3.0±2.7%, n=6; psychLight2KETSN=−3.5±1.6%), n=3. ****p<0.0001, **p<0.01 and *p<0.05, one-way ANOVA compared to 5-HT with Dunnett's test. D. Bar graph showing PSYLI2 response to 10 mM 5-HT (44.8±0.8%) and 10 mM KETSN (−12.7±0.5%), and frequency distribution of both positive (magenta, 5-HT) and negative (blue, KETSN) control treatments (Z-factorPSYLI2=0.6), n=42. ****p<0.0001, unpaired t test.

FIG. 20. Characterization of PSYLI2 cells and their use in high content screening. A. Schematic depicting the workflow for a screening campaign using both agonist and antagonist modes. B. Agonist mode screen of a compound library using PSYLI2 cells. C. Antagonist mode screen of a compound library using PSYLI2 cells. D. Concentration-response studies using PSYL2 cells (top) and a cell free wells (bottom) reveal that BOL-148 and bromocriptine produce fluorescence artifacts at 10 mM (****p<0.0001, ***p<0.001, **p<0.01 and *p<0.05, one-way ANOVA compares to parent variant within the group with Dunnett's test). Data are represented as mean±SEM 5-HT=5-hydroxytryptamine; KETSN=ketanserin; BOL-148=2-bromolysergic acid diethylamide.

FIG. 21. Chemical structures of compounds synthesized in house. Syntheses and characterization data for compounds are shown in the supporting information.

FIG. 22. Schild regression analysis for non-hallucinogenic 5-HT2AR ligands and off target characterization for AAZ-A-154. A-D. Schild analysis using 5-HT (100 nM) as the agonist and 4 concentrations of non-hallucinogenic compounds (i.e., LIS, 6-MeO, apomorphine, benztropine). LIS=lisuride, 6-MeO=6-MeO-DMT, APOM=apomorphine, BZTP=benztropine. E. Data from agonist and antagonist mode of 7 receptor-based sensors (Agonist mode: 0.1% DMSO or 10 mM AAZ-A-154 was added; Antagonist mode: 0.1% DMSO or 10 uM AAZ-A-154 was added in the presence of 100 nM dopamine (DA), norepinephrine (NE), U-50488 (U50) or serotonin(5-HT)). ****p<0.0001, ns p>0.05. One-way ANOVA. Tukey's multiple comparisons test.

FIG. 23 shows the sequence of PsychLight1 (SEQ ID NO: 52).

FIG. 24 shows the sequence of PsychLight2 (SEQ ID NO: 53).

### DETAILED DESCRIPTION OF THE INVENTION

### I. General

The present invention provides fluorescent biosensors for detection of a hallucinogenic compound.

### II. Definitions

Unless specifically indicated otherwise, all technical and scientific terms used herein have the same meaning as commonly understood by those of ordinary skill in the art to which this invention belongs. In addition, any method or material similar or equivalent to a method or material described herein can be used in the practice of the present invention. For purposes of the present invention, the following terms are defined.

“A,” “an,” or “the” refers to not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

The terms “identical” or percent “identity,” in the context of two or more nucleic acids or polypeptide sequences, refer to two or more sequences or subsequences that are the same or have a specified percentage of amino acid residues or nucleotides that are the same (i.e., share at least about 80% identity, for example, at least about 85%, 90%, 91%, 92%, 93%, 94%, 95%, 96%, 97%, 98%, 99% or 100% identity over a specified region to a reference sequence, e.g., any of SEQ ID NOs: 1-44, as described herein, when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, for example, over a region that is 50, 100, 200, 300, 400 amino acids or nucleotides in length, or over the full-length of a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins to fluorescent proteins, circularly permuted fluorescent proteins, and GPCR nucleic acids and proteins, the BLAST and BLAST 2.0 algorithms and the default parameters are used.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “isolated,” and variants thereof when applied to a protein (e.g., a population of GPCRs having an integrated cpFP sensor), denotes that the protein is essentially free of other cellular components with which it is associated in the natural state. It is preferably in a homogeneous state. It can be in either a dry or aqueous solution, or solubilized. Purity and homogeneity are typically determined using known techniques, such as polyacryl amide gel electrophoresis or high performance liquid chromatography. A protein that is the predominant species present in a preparation is substantially purified.

The term “purified” denotes that a protein (e.g., a population of GPCRs having an integrated cpFP sensor) gives rise to essentially one band in an electrophoretic gel. Particularly, it means that the nucleic acid or protein is at least 80%, 85% or 90% pure, more preferably at least 95% pure, and most preferably at least 99% pure.

“Agonism” refers to the activation of a receptor or enzyme by a modulator, or agonist, to produce a biological response.

“Agonist” refers to a modulator that binds to a receptor or enzyme and activates the receptor to produce a biological response. By way of example only, “5HT_{2A} agonist” can be used to refer to a compound that exhibits an EC₅₀ with respect to 5HT_{2A} activity of no more than about 100 μM. In some embodiments, the term “agonist” includes full agonists or partial agonists. “Full agonist” refers to a modulator that binds to and activates a receptor with the maximum response that an agonist can elicit at the receptor. “Partial agonist” refers to a modulator that binds to and activates a given receptor, but has partial efficacy, that is, less than the maximal response, at the receptor relative to a full agonist.

“Positive allosteric modulator” refers to a modulator that binds to a site distinct from the orthosteric binding site and enhances or amplifies the effect of an agonist.

“Antagonism” refers to the inactivation of a receptor or enzyme by a modulator, or antagonist. Antagonism of a receptor, for example, is when a molecule binds to the receptor and does not allow activity to occur.

“Antagonist” or “neutral antagonist” refers to a modulator that binds to a receptor or enzyme and blocks a biological response. An antagonist has no activity in the absence of an agonist or inverse agonist but can block the activity of either, causing no change in the biological response.

“Change in fluorescence” refers to an increase or decrease in the intensity or wavelength of the emitted light for a compound following exposure to light having a shorter wavelength. For example, the change in intensity can be an increase or decrease of 1% to more than 100%. A change in wavelength for fluorescence can be from about 1 nm to more than 500 nm. Fluorescent wavelengths are typically between 250 and 700 nm, so a change in fluorescence can be from one wavelength between 250 and 700 nm to another wavelength between 250 and 700 nm.

“Hallucinogenic compound” or “hallucinogen” refers to a compound causing hallucinations in a subject.

“Hallucinogenic potential” refers to the ability of a compound to induce changes in perception characteristic of classic serotonergic psychedelics such as LSD and psilocybin.

“Antipsychotic potential” refers to the ability of a compound to treat one or more psychotic disorders known to one of skill in the art.

### III. FLUORESCENT BIOSENSOR

PCT Publication No. WO2018/098262 is incorporated herein by reference in its entirety for all purposes.

1. Fluorescent Sensors

Provided are fluorescent sensors designed to integrate into the third intracellular loop of a G protein-coupled receptor (GPCR). In some embodiments, the sensors comprise the following polypeptide structure: L1-cpFP-L2, wherein:
(1) L1 comprises a peptide linker having LSS at the N-terminus and from 5 to 13 amino acid residues, wherein each amino acid residue can be any naturally occurring amino acid;
(2) cpFP comprises a circularly permuted fluorescent protein, wherein the circularly permuted N-terminus is positioned within beta strand seven of a non-permuted fluorescent protein; and
(3) L2 comprises a peptide linker having DQL at the C-terminus and from 5 to 6 amino acid residues, wherein each amino acid residue can be any naturally occurring amino acid.

Generally, the fluorescent sensors are integrated into a GPCR, e.g., into the third intracellular loop. The GPCR internal fluorescent sensors are polypeptides that can be produced using any method known in the art, including synthetic and recombinant methodologies. When produced recombinantly, the GPCR internal fluorescent sensor polypeptides can be expressed in eukaryotic or prokaryotic host cells.

a. Circularly Permuted Fluorescent Protein

The circularly permuted fluorescent protein (cpFP) can be from any known fluorescent protein known in the art. In some embodiments, the circularly permuted protein is from a green fluorescent protein (GFP) or a red fluorescent protein (RFP), e.g., from mCherry, mEos2, mRuby2, mRuby3, mClover3, mApple, mKate2, mMaple, mCardinal, mNeptune, far-red single-domain cyanbacteriochrome WP_016871037 or far-red single-domain cyanbacteriochrome anacy 2551g3. Generally, the N-terminus of the circularly permuted is an amino acid residue within the seventh beta strand of the fluorescent protein in its non-circularly permuted form. Within the seventh beta strand of the fluorescent protein, in some embodiments, the circularly permuted N-terminus of the cpFP is positioned within the motif YN(Y/F)(N/I)SHNV, e.g., of a non-permuted green fluorescent protein, or within the motif WE(A/P/V)(S/L/N/T)(S/E/T)E(R/M/T/K)(M/L) of a non-permuted red fluorescent protein. In some embodiments, the circularly permuted N-terminus is positioned at the amino acid residue corresponding to residue 7 (e.g., N) of the amino acid motif YN(Y/F)(N/I)SHNV of a non-permuted green fluorescent protein. In some embodiments, the circularly permuted N-terminus is positioned at the amino acid residue corresponding to residue 3 (e.g., (A/P/U/V/P)), 4 (e.g., (LSN)), 5 (e.g., S/T)), 6 (e.g., E) or 7 (e.g., R/M/K/T)) of the amino acid motif WE(A/P/V)(S/L/N/T)(S/E/T)E(R/M/T/K)(M/L) of a non-permuted red-fluorescent protein.

In some embodiments, the circularly permuted fluorescent protein is from a photo-convertible or photoactivable fluorescent protein. Numerous photo-convertible or photoactivable fluorescent proteins are known in the art, and their circularly permuted forms can be used in the present sensors. See, Rodriguez, et al., Trends Biochem Sci. (2016) Nov. 1. pii: S0968-0004(16)30173-6; Ai, et al., Nat Protoc. 2014 April; 9(4):910-28; Kyndt, et al., Photochem Photobiol Sci. 2004 June; 3(6):519-30; Meyer, et al., Photochem Photobiol Sci. 2012 October; 11(10):1495-514. In some embodiments, the photo-convertible or photoactivable fluorescent protein is selected from the group consisting of photoactivable green fluorescent protein (paGFP), mCherry, mEos2, mRuby2, mRuby3, mClover3, mApple, mKate2, mMaple, far-red single-domain cyanbacteriochrome WP_016871037 and far-red single-domain cyanbacteriochrome anacy 2551g3.

In some embodiments, the circularly permuted fluorescent protein is from a fluorescent protein having at least about 90% sequence identity, e.g., at least about 91%, 92%, 93%, 94%, 95%, 96%, 97%, 98%, 99% or 100% sequence identity, to a non-permuted fluorescent protein selected from the group consisting of SEQ ID NOs: 1-14. In some embodiments, the circularly permuted fluorescent protein is from a green fluorescent protein having at least about 90% sequence identity, e.g., at least about 91%, 92%, 93%, 94%, 95%, 96%, 97%, 98%, 99% or 100% sequence identity, to SEQ ID NO: 1, wherein the tyrosine at residue position 69 of SEQ ID NO:1 is replaced with a tryptophan (Y69W) to generate a cyan fluorescent protein (CFP) sensor. In some embodiments, the circularly permuted fluorescent protein is from a green fluorescent protein having at least about 90% sequence identity, e.g., at least about 91%, 92%, 93%, 94%, 95%, 96%, 97%, 98%, 99% or 100% sequence identity, to SEQ ID NO: 1, wherein the threonine at residue position 206 of SEQ ID NO:1 is replaced with a tyrosine (T206Y) to generate a yellow fluorescent protein (YFP) sensor. In some embodiments, the circularly permuted fluorescent protein has at least about 90% sequence identity, e.g., at least about 91%, 92%, 93%, 94%, 95%, 96%, 97%, 98%, 99% or 100% sequence identity, to a circularly permuted fluorescent protein selected from the group consisting of SEQ ID NOS: 15-18.

Numerous circularly permuted fluorescent proteins are described in the art, and may find use in the present fluorescent sensors. The choice of a particular circularly permuted fluorescent protein for use in a fluorescent protein sensor may depend on the desired emission spectrum for detection, and include, but is not limited to, circularly permuted fluorescent proteins with green, blue, cyan, yellow, orange, red, or far-red emissions. A number of circularly permuted fluorescent proteins are known and can be used in the present sensors. See, e.g., Pedelacq et al. (2006) Nat. Biotechnol. 24:79-88 for a description of circularly permuted superfolder GFP variant (cpsfGFP), Zhao et al. (2011) Science 333:1888-1891 for a description of circularly permuted mApple; Shui et al. (2011) PLoS One; 6(5):e20505 for a description of circularly permuted variants of mApple and mKate; Carlson et al. (2010) Protein Science 19:1490-1499 for a description of circularly permuted red fluorescent proteins, Gautam et al. (2009) Front. Neuroeng. 2:14 for a description of circularly permuted variants of enhanced green fluorescent protein (EGFP) and mKate, Zhao et al. (2011) Science 333(6051):1888-1891 for a description of a circularly permuted variant of mApple; Liu et al. (2011) Biochem. Biophys. Res. Commun. 412(1):155-159 for a description of circularly permuted variants of Venus and Citrine, Li et al. (2008) Photochem. Photobiol. 84(1):111-119 for a description of circularly permuted variants of mCherry, and Perez-Jimenez et al. (2006) J. Biol. Chem. December 29; 281(52):40010-40014 for a description of circularly permuted variants of enhanced yellow fluorescent protein (EYFP). Further illustrative circularly permuted fluorescent proteins are described in e.g., Honda, et al., PLoS One. 2013 May 22; 8(5):e64597; Schwartzlander, et al., Biochem J. 2011 Aug. 1; 437(3):381-7; Miyawaki, et al., Adv Biochem Eng Biotechnol. 2005; 95:1-15; Tantama, et al., Prog Brain Res. 2012; 196:235-63; Mizuno, et al., J Am Chem Soc. 2007 Sep. 19; 129(37):11378-83; Chiang, et al., Biotechnol Lett. 2006 April; 28(7):471-5; and in U.S. Patent Publication Nos. 2015/0132774; 2010/0021931; and 2008/0178309.

b. N-Terminal and C-Terminal Linkers

The G protein-coupled receptor (GPCR) internal fluorescent sensors have an N-terminal linker (L1) and a C-terminal linker (L2). In some embodiments, L1 comprises a peptide linker having from 2 to 13 amino acid residues, e.g., 2 to 3, 4, 5, 6, 7, 8, 9, 10, 11, 12 or 13 residues, wherein each amino acid residue can be any naturally occurring amino acid. In some embodiments, L2 comprises a peptide linker having from 2 to 5 amino acid residues, e.g., 2 to 3, 4 or 5 residues, wherein each amino acid residue can be any naturally occurring amino acid. In some embodiments, L1 and L2 are peptides that independently have 2, 3, 4, 5, or 6 amino acid residues. In some embodiments, L1 comprises LSSLI and L2 comprises NHDQL. In some embodiments, L1 comprises LSSXIX2 and L2 comprises X3X4DQL, wherein X1, X2, X3, X4 are independently any amino acid. In some embodiments, L1 comprises QLQKIDLSSXlX2 and L2 comprises X3X4DQL, wherein X1, X2, X3, X4 are independently any amino acid. In some embodiments, X1X2 is selected from the group consisting of leucine-isoleucine (LI), alanine-valine (AV), isoleucine-lysine (IK), serine-arginine (SR), lysine-valine (KV), leucine-alanine (LA), cysteine-proline (CP), glycine-methionine (GM), valine-arginine (VR), asparagine-valine (NV), arginine-valine (RV), arginine-glycine (RG), leucine-glutamate (LE), serine-glycine (SG), valine-aspartate (VD), alanine-phenylalanine (AF), threonine-aspartate (TD), methionine-arginine (MR), leucine-glycine (LG), arginine-glutamine (RQ), serine-tryptophan (SW), serine-glycine (SG), valine-aspartate (VD), leucine-glutamate (LE), alanine-phenylalanine (AF), serine-tryptophan (SW), arginine-glycine (RG), threonine-aspartate (TD), leucine-glycine (LG), arginine-glutamine (RQ), threonine-tyrosine (TY), leucine-leucine (LL), valine-leucine (VL), threonine-glutamine (TQ), valine-phenylalanine (VF), threonine-threonine (TT), leucine-valine (LV), valine-isoleucine (VI), valine-valine (VV), proline-valine (PV), glycine-valine (GV), serine-valine (SV), phenylalanine-valine (FV), cysteine-valine (CV), glutamate-valine (EV), glutamine-valine (QV), and lysine-valine (KV), arginine-tryptophan (RW), glycine-aspartate (GD), alanine-leucine (AL), proline-methionine (PM), glycine-arginine (GR), glycine-tyrosine (GY), isoleucine-cysteine (IC), and glycine-leucine (GL). In some embodiments, X3X4 is selected from the group consisting of asparagine-histidine (NH), threonine-arginine (TR), isoleucine-isoleucine (II), proline-proline (PP), leucine-phenylalanine (LF), valine-threonine (VT), glutamine-glycine (QG), alanine-leucine (AL), proline-arginine (PR), arginine-glycine (RG), threonine-leucine (TL), threonine-proline (TP), glycine-valine (GV), threonine-threonine (TT), cysteine-cysteine (CC), alanine-threonine (AT), leucine-proline (LP), tyrosine-proline (YP), tryptophan-proline (WP), serine-leucine (SL), glutamate-arginine (ER), methionine-cysteine (MC), methionine-histidine (MH), tryptophan-leucine (YL), leucine-serine (LS), arginine-proline (RP), lysine-proline (KP), tyrosine-proline (YP), tryptophan-proline (WP), serine-serine (SS), glycine-valine (GV), valine-serine (VS), glutamine-asparagine (QN), lysine-serine (KS), lysine-threonine (KT), lysine-histidine (KH), lysine-valine (KV), lysine-glutamine (KQ), lysine-arginine (KR), cysteine-proline (CP), alanine-proline (AP), serine-proline (SP), isoleucine-proline (IP), tyrosine-proline (YP), threonine-proline (TP), arginine-proline (RP), aspartate-histidine (DH), histidine-tyrosine (HY), glycine-glycine (GG), proline-histidine (PH), serine-threonine (ST), arginine-serine (RS), arginine-histidine (RH), and tryptophan-proline (WP). In some embodiments, X1X2 comprises alanine-valine (AV) and X3X4 comprises lysine-proline (KP); threonine-arginine (TR); aspartate-histidine (DH); threonine-threonine (TT); serine-serine (SS); glycine-valine (GV); cysteine-cysteine (CC); valine-serine (VS); glutamine-asparagine (QN); lysine-serine (KS); lysine-threonine (KT); lysine-histidine (KH); lysine-valine (KV); lysine-glutamine (KQ); lysine-arginine (KR); lysine-proline (KP); cysteine-proline (CP); alanine-proline (AP); serine-proline (SP); isoleucine-proline (IP); tyrosine-proline (YP); threonine-proline (TP); or arginine-proline (RP); XTX2 comprises leucine-valine (LV) and X3X4 comprises threonine-arginine (TR), lysine-proline (KP) or valine-threonine (VT); X1X2 comprises arginine-valine (RV) and X3X4 comprises threonine-arginine (TR), lysine-proline (KP) or threonine-proline (TP); XTX2 comprises arginine-glycine (RG) and X3X4 comprises tyrosine-leucine (YL) or threonine-arginine (TR); XTX2 comprises serine-arginine (SR) and X3X4 comprises leucine-phenylalanine (LF) or proline-proline (PP); XTX2 comprises proline-methionine (PM) and X3X4 comprises proline-histidine (PH) or serine-serine (SS); XTX2 comprises valine-valine (VV) and X3X4 comprises threonine-arginine (TR) or lysine-proline (KP); XTX2 comprises leucine-isoleucine (LI) and X3X4 comprises threonine-arginine (TR); XTX2 comprises threonine-tyrosine (TY) and X3X4 comprises threonine-arginine (TR); XTX2 comprises isoleucine-lysine (IK) and X3X4 comprises isoleucine-isoleucine (II); X1X2 comprises cysteine-proline (CP) and X3X4 comprises alanine-leucine (AL); XTX2 comprises glycine-methionine (GM) and X3X4 comprises proline-arginine (PR); X1X2 comprises leucine-alanine (LA) and X3X4 comprises glutamine-glycine (QG); X1X2 comprises valine-arginine (VR) and X3X4 comprises arginine-glycine (RG); X1X2 comprises serine-glycine (SG) and X3X4 comprises tyrosine-proline (YP); X1X2 comprises valine-aspartate (VD) and X3X4 comprises tryptophan-proline (WP); X1X2 comprises leucine-glutamate (LE) and X3X4 comprises leucine-proline (LP); X1X2 comprises alanine-phenylalanine (AF) and X3X4 comprises serine-leucine (SL); X1X2 comprises serine-tryptophan (SW) and X3X4 comprises arginine-proline (RP); X1X2 comprises threonine-aspartate (TD) and X3X4 comprises glutamate-arginine (ER); X1X2 comprises leucine-glycine (LG) and X3X4 comprises methionine-histidine (MH); X1X2 comprises arginine-glutamine (RQ) and X3X4 comprises leucine-serine (LS); X1X2 comprises methionine-arginine (MR) and X3X4 comprises methionine-cysteine (MC); X1X2 comprises leucine-leucine (LL) and X3X4 comprises threonine-arginine (TR); X1X2 comprises valine-leucine (VL) and X3X4 comprises threonine-arginine (TR); X1X2 comprises threonine-glutamine (TQ) and X3X4 comprises threonine-arginine (TR); X1X2 comprises valine-phenylalanine (VF) and X3X4 comprises threonine-arginine (TR); X1X2 comprises threonine-threonine (TT) and X3X4 comprises threonine-arginine (TR); X1X2 comprises valine-isoleucine (VI) and X3X4 comprises threonine-arginine (TR); X1X2 comprises proline-valine (PV) and X3X4 comprises lysine-proline (KP); X1X2 comprises glycine-valine (GV) and X3X4 comprises lysine-proline (KP); X1X2 comprises serine-valine (SV) and X3X4 comprises lysine-proline (KP); X1X2 comprises asparagine-valine (NV) and X3X4 comprises lysine-proline (KP); X1X2 comprises phenylalanine-valine (FV) and X3X4 comprises lysine-proline (KP); X1X2 comprises cysteine-valine (CV) and X3X4 comprises lysine-proline (KP); X1X2 comprises glutamate-valine (EV) and X3X4 comprises lysine-proline (KP); X1X2 comprises glutamine-valine (QV) and X3X4 comprises lysine-proline (KP); X1X2 comprises lysine-valine (KV) and X3X4 comprises lysine-proline (KP); X1X2 comprises arginine-tryptophan (RW) and X3X4 comprises histidine-tyrosine (HY); X1X2 comprises glycine-aspartate (GD) and X3X4 comprises glycine-glycine (GG); X1X2 comprises alanine-leucine (AL) and X3X4 comprises asparagine-histidine (NH); X1X2 comprises glycine-arginine (GR) and X3X4 comprises serine-threonine (ST); X1X2 comprises glycine-tyrosine (GY) and X3X4 comprises arginine-serine (RS); X1X2 comprises isoleucine-cysteine (IC) and X3X4 comprises arginine-histidine (RH); or X1X2 comprises glycine-leucine (GL) and X3X4 comprises tryptophan-proline (WP). In some embodiments, L1 comprises LSSLIX1 and L2 comprises X2NHDQL, wherein X1, X2 are independently any amino acid. In some embodiments, X1 is selected from the group consisting of I, W, V, L, F, P, N, Y and D; and X2 is selected from the group consisting of G, N, M, R T, S, K, L, Y, H, F, E, I and W. In some embodiments, X1 is I and X2 is N or S; X1 is W and X2 is M, T, F, E or I; X1 is V and X2 is R, H or T; X1 is L and X2 is T; X1 is F and X2 is S; X1 is P and X2 is K or S; X1 is Y and X2 is S, L; or X1 is D and X2 is W.

2. G Coupled Protein Receptors with Integrated Sensors

In some embodiments, the fluorescent sensors are incorporated or integrated into the third intracellular loop of a G protein-coupled receptor (GPCR). This can be readily accomplished employing recombinant techniques known in the art. Generally, any amino acid within the third loop region of a GPCR may serve as an insertion site for a cpFP (e.g., before or after, or as a replacement). In some embodiments, the cpFP sensor is inserted between two amino acid residues within the middle third of the third intracellular loop of a G protein-coupled receptor (GPCR). As necessary or appropriate, one, two, three, four, or more, amino acid residues within the third intracellular loop of the wild-type G protein-coupled receptor may be removed in order that the loop can accommodate the sensor. In some embodiments for inserting a cpFP into the third intracellular loop, the third intracellular loop and part of the sixth transmembrane sequence (TM6) (e.g., for a beta2 adrenergic receptor RQLQ - - - cpFP - - - CWLP) can be used as a module system to transfer to other GPCRs.

As is standard or customary in the art, the “third intracellular loop” or “third cytoplasmic loop” is with reference to N-terminus of the GPCR that is integrated into the extracellular membrane of a cell and refers to the third segment of a GPCR polypeptide that is located in the cytoplasmic or intracellular side of the extracellular membrane. It is phrase commonly used by those of skill in the art. See, e.g., Kubale, et al., Int J Mol Sci. (2016) Jul. 19;17(7); Clayton, et al., J Biol Chem. (2014) Nov. 28; 289(48):33663-75; Gómez-Moutón, et al., Blood. (2015) Feb. 12; 125(7):1116-25; Terawaki, et al., Biochem Biophys Res Commun. 2015 Jul. 17-24; 463(1-2):64-9; Gabl, et al., PLoS One. 2014 Oct. 10; 9(10):e109516; Fukunaga, et al., Mol Neurobiol. 2012 February; 45(1):144-52; Nakatsuma, et al., Biophys J. 2011 Apr. 20; 100(8):1874-82; Shioda, et al., J Pharmacol Sci. 2010; 114(1):25-31; Shpakov, et al., Dokl Biochem Biophys. 2010 March-Apr; 431:94-7; Takeuchi, et al., J Neurochem. 2004 June; 89(6):1498-507.

Accordingly, provided are G protein-coupled receptors comprising a cpFP sensor, as described above and herein, wherein the sensor is integrated into the third intracellular loop of the G protein-coupled receptor.

In some embodiments, the G protein-coupled receptor is a class A type or alpha G protein-coupled receptor. In some embodiments, the G protein-coupled receptor is selected from the group consisting of an adrenoceptor or adrenergic receptor, an opioid receptor, a 5-Hydroxytryptamine (5-HT) receptor, a dopamine receptor, a muscarinic acetylcholine receptor, an adenosine receptor, a glutamate metabotropic receptor, a gamma-aminobutyric acid (GABA) type B receptor, corticotropin-releasing factor (CRF) receptor, a tachykinin or neurokinin (NK) receptor, an angiotensin receptor, an apelin receptor, a bile acid receptor, a bombesin receptor, a bradykinin receptor, a cannabinoid receptor, a chemokine receptor, a cholecystokinin receptor, a complement peptide receptor, an endothelin receptor, a formylpeptide receptor, a free fatty acid receptor, a galanin receptor, a ghrelin receptor, a glycoprotein hormone, a gonadotrophin-releasing hormone receptor, a G protein-coupled estrogen receptor, an histamine receptor, a leukotriene receptor, a lysophospholipid (LPA) receptor, a lysophospholipid (SIP) receptor, a melanocortin receptor, a melatonin receptor, a neuropeptide receptor, a neurotensin receptor, an orexin receptor, a P2Y receptor, a prostanoid or prostaglandin receptor, somatostatin receptor, a tachykinin receptor, a thyrotropin-releasing hormone receptor, a urotensin receptor, and a vasopressin/oxytocin receptor. In some embodiments, the G protein-coupled receptor is selected from the group consisting of an adrenoceptor beta 1 (ADRB1), adrenoceptor beta 2 (ADRB2), adrenoceptor alpha 2A (ADRA2A), a mu (μ)-type opioid receptor (OPRM), a kappa (κ)-type opioid receptor (OPRK), a delta (δ)-type opioid receptor (OPRD), a dopamine receptor D1 (DRD1), a 5-hydroxy-tryptamine receptor 2A (5-HT2A), a melatonin receptor type 1B (MTNR1B), an adenosine A1 receptor (ADORA1), a cannabinoid receptor (type-1) (CNR1), a histamine receptor H1 (HRH1), a neuropeptide Y receptor Y1 (NPY1R), a cholinergic receptor muscarinic 2 (CHRM2), a hypocretin (orexin) receptor 1 (HCRTR1), a tachykinin receptor 1 (TACR1) (a.k.a. neurokinin 1 receptor (NK1R)), a corticotropin releasing hormone receptor 1 (CRHRI), a glutamate metabotropic receptor 1 (GRM1), and a gamma-aminobutyric acid (GABA) type B receptor subunit 1 (GABBRI). In some embodiments, the G protein-coupled receptor is selected from the group consisting of: Metabotropic Glutamate Receptor type-3 (MGLUR3); Metabotropic Glutamate Receptor type-5 (MGLUR5); Gamma-aminobutyric acid Receptor type-2 (GABAB1); Gamma-aminobutyric acid Receptor type-2 (GABAB2); Cannabinoid Receptor type-1 (CB1); Gonadotropin-Releasing Hormone Receptor (GNRHR); Vasopressin Receptor type-1 (VIA); Oxytocin Receptor (OTR); Adenosine Receptor type-2 (A2A); Beta-2 Adrenergic Receptor (B2AR); Dopamine Receptor type-1 (DRD1); Dopamine Receptor type-2 (DRD2); Acetylcholine Muscarinic Receptor type-2 (M2R); Histamine Receptor type-1 (H1R); Serotonin Receptor type-2A (5HT2A); Serotonin Receptor type-2B (5HT2B); Tachykinin Receptor type-1 (NK1); Tachykinin Receptor type-2 (NK2); Tachykinin Receptor type-3 (NK3); Melatonin Receptor type-1B (MTNRIB); P2 purinoceptor type Y1 (P2Y1); Angiotensin-II Receptor type-1 (AT1); Kappa Opioid Receptor type-1 (KORI); Mu Opioid Receptor type-1 (MOR1); and Delta Opioid Receptor type-1 (DOR1).

In some embodiments, the receptor is mutated to be signaling incompetent or incapable. To prevent internalization and arrestin-dependent signaling for any GPCR, GRK6 phosphorylation sites can be replaced with alanine residues. The residue numbers and location of the G protein-coupled receptor kinase 6 (GRK6) residues vary between different GPCRs. On the Beta2AR, the GRK6 residues are SS355, 356 (residues 624-625 of SEQ ID NO: 22). Alternatively or additionally, G-protein dependent signaling can be prevented or inhibited by mutating a specific residue that is mostly conserved among many GPCRs. This residue corresponds to Phenylalanine (F) 139 (residue F163 of SEQ ID NO: 22) on the Beta2AR. This conserved residue that facilitates G protein dependent signaling varies from GPCR to GPCR.

In some embodiments, the G protein-coupled receptor comprising an integrated cpFP sensor comprises a beta2 adrenergic receptor having at least 90% sequence identity, e.g., at least about 91%, 92%, 93%, 94%, 95%, 96%, 97%, 98%, 99% or 100% sequence identity, to SEQ ID NO: 22 or SEQ ID NO:32. In some embodiments, the sensor replaces one or more or all of amino acid residues QLQKIDKSEGRFHVQNLS (residues 253-270 of SEQ ID NO:22) and the carboxy-terminus of L2 abuts KEHK (residues 536-539 of SEQ ID NO:22). In some embodiments, the sensor replaces one or more or all of amino acid residues QLQKIDKSEGRFHVQNLS (residues 253-270 of SEQ ID NO:22) and the carboxy-terminus of L2 abuts FCLK (residues 533-536 of SEQ ID NO:22). In some embodiments, one or more of amino acid residues F139, S355 and S356 (residues 163 and 624-625 in SEQ ID NO: 22) of the beta2 adrenergic receptor are replaced with alanine residues to render the beta2 adrenergic receptor signaling incompetent. In some embodiments, X at amino acid residue 163 in SEQ ID NO: 22 or at residue 139 of SEQ ID NO:32 is any amino acid or an amino acid selected from the group consisting of A, F, G, I, L, M, S, T and V, particularly A. In some embodiments when the G protein-coupled receptor is a beta2 adrenergic receptor, the cpFP sensor is inserted into the third intracellular loop between residues AKRQ and LQKI, e.g., between residues 253 and 254 of SEQ ID NO:22. In some embodiments, the insertion sites of the cpGFP into a beta2 adrenergic receptor can be any amino acids in the region of KSEGRFHVQLSQVEQDGRTGHGL of the third loop. In some embodiments when the G protein-coupled receptor is a beta2 adrenergic receptor, the cpFP sensor is inserted into the third intracellular loop between residues QNLS and AEVK, e.g., between residues 270 and 271 of SEQ ID NO:22. In some embodiments when the G protein-coupled receptor is a beta2 adrenergic receptor, the cpFP sensor is inserted into the third intracellular loop between residues EAKR and QLQK, e.g., between residues 252 and 253 of SEQ ID NO:22. In some embodiments when the G protein-coupled receptor is a beta2 adrenergic receptor, the cpFP sensor is inserted into the third intracellular loop between residues KRQL and QKID, e.g., between residues 254 and 255 of SEQ ID NO:22. In some embodiments when the G protein-coupled receptor is a beta2 adrenergic receptor, L1 of the cpFP sensor is alanine-valine (AV) and L2 of the cpFP sensor is threonine-arginine (TR) or lysine-proline (KP).

In some embodiments, the G protein-coupled receptor comprising an integrated cpFP sensor comprises a mu (μ)-type opioid receptor having at least 90% sequence identity, e.g., at least about 91%, 92%, 93%, 94%, 95%, 96%, 97%, 98%, 99% or 100% sequence identity, to SEQ ID NO:24 or SEQ ID NO:37. In some embodiments, amino acid residue V199 (residue 199 in SEQ ID NO: 24) of the mu (μ)-type opioid receptor is replaced with an alanine residue to render the mu (μ)-type opioid receptor signaling incompetent. In some embodiments, X at amino acid residue 199 in SEQ ID NO: 24 or at residue 175 of SEQ ID NO:37 is any amino acid or an amino acid selected from the group consisting of A, F, G, I, L, M, S, T and V, particularly A. In some embodiments when the G protein-coupled receptor is a mu (μ)-type opioid receptor, the cpFP sensor is inserted into the third intracellular loop between residues RMLS and GS, e.g., between residues 292 and 293 of SEQ ID NO:24. In some embodiments when the G protein-coupled receptor is a mu (μ)-type opioid receptor, L1 of the cpFP sensor is isoleucine-lysine (IK) and L2 of the cpFP sensor is isoleucine-isoleucine (II).

In some embodiments, the G protein-coupled receptor comprising an integrated cpFP sensor comprises a dopamine receptor D1 (DRD1) having at least 90% sequence identity, e.g., at least about 91%, 92%, 93%, 94%, 95%, 96%, 97%, 98%, 99% or 100% sequence identity, to SEQ ID NO: 26 or SEQ ID NO:30. In some embodiments, the N-terminus of L1 abuts IAQK (residues 244-247 of SEQ ID NO:26), the C-terminus of L2 abuts KRET (residues 534-537 of SEQ ID NO:26), the sensor replacing residues 248 to 533 of SEQ ID NO:26. In some embodiments, amino acid residue F129 (residue 153 in SEQ ID NO: 26 or residue 129 of SEQ ID NO:30) of the dopamine receptor D1 (DRD1) is replaced with an alanine residue to render the dopamine receptor D1 (DRD1) signaling incompetent. In some embodiments, X at amino acid residue 153 in SEQ ID NO: 26 or at residue 129 of SEQ ID NO:30 is any amino acid or an amino acid selected from the group consisting of A, F, G, I, L, M, S, T and V, particularly A. In some embodiments when the G protein-coupled receptor is a dopamine receptor D1 (DRD1), the cpFP sensor is inserted into the third intracellular loop between residues AKNC and QTTT, e.g., between residues 265 and 266 of SEQ ID NO:21. In some embodiments when the G protein-coupled receptor is a dopamine receptor D1 (DRD1), L1 of the cpFP sensor is serine-arginine (SR) and L2 of the cpFP sensor is proline-proline (PP).

In some embodiments, the G protein-coupled receptor comprising an integrated cpFP sensor comprises a 5 hydroxy-tryptamine 2A (5-HT2A) receptor having at least 90% sequence identity, e.g., at least about 91%, 92%, 93%, 94%, 95%, 96%, 97%, 98%, 99% or 100% sequence identity, to SEQ ID NO: 28 or SEQ ID NO:33. In some embodiments, the N-terminus of L1 abuts SLQK (residues 284-287 of SEQ ID NO:28), the C-terminus of L2 abuts NEQK (residues 586-589 of SEQ ID NO:28), the sensor replacing residues 288 to 585 of SEQ ID NO:28. In some embodiments, amino acid residue 1181 (residue 205 in SEQ ID NO: 28) of the 5-hydroxy-tryptamine 2A (5-HT2A) receptor is replaced with an alanine residue to render the 5-hydroxy-tryptamine 2A (5-HT2A) receptor signaling incompetent. In some embodiments, X at amino acid residue 205 in SEQ ID NO: 28 or at residue 181 of SEQ ID NO:33 is any amino acid or an amino acid selected from the group consisting of A, F, G, I, L, M, S, T and V, particularly A. In some embodiments when the G protein-coupled receptor is a 5-hydroxy-tryptamine 2A (5-HT2A) receptor, the cpFP sensor is inserted into the third intracellular loop between residues TRAK and LASF, e.g., between residues 301 and 302 of SEQ ID NO:23. In some embodiments when the G protein-coupled receptor is a 5-hydroxy-tryptamine 2A (5-HT2A) receptor, L1 of the cpFP sensor is serine-arginine (SR) and L2 of the cpFP sensor is leucine-phenylalanine (LF).

In some embodiments, the G protein-coupled receptor comprising an integrated cpFP sensor comprises an adrenoceptor beta 1 (ADRB1) having at least 90% sequence identity, e.g., at least about 91%, 92%, 93%, 94%, 95%, 96%, 97%, 98%, 99% or 100% sequence identity, to SEQ ID NO:31. In some embodiments, X at amino acid residue 164 in SEQ ID NO: 31 is any amino acid or an amino acid selected from the group consisting of A, F, G, I, L, M, S, T and V, particularly A. In some embodiments, the G protein-coupled receptor comprising an integrated cpFP sensor comprises an adenosine A2a receptor (ADORA2A) having at least 90% sequence identity, e.g., at least about 91%, 92%, 93%, 94%, 95%, 96%, 97%, 98%, 99% or 100% sequence identity, to SEQ ID NO: 34. In some embodiments, X at amino acid residue 110 in SEQ ID NO: 34 is any amino acid or an amino acid selected from the group consisting of A, F, G, I, L, M, S, T and V, particularly A. In some embodiments, the G protein-coupled receptor comprising an integrated cpFP sensor comprises an adrenoceptor alpha 2A (ADRA2A) having at least 90% sequence identity, e.g., at least about 91%, 92%, 93%, 94%, 95%, 96%, 97%, 98%, 99% or 100% sequence identity, to SEQ ID NO: 35. In some embodiments, X at amino acid residue 139 in SEQ ID NO: 35 is any amino acid or an amino acid selected from the group consisting of A, F, G, I, L, M, S, T and V, particularly A. In some embodiments, the G protein coupled-receptor comprising an integrated cpFP sensor comprises a kappa receptor delta 1 (OPRKI1) having at least 90% sequence identity, e.g., at least about 91%, 92%, 93%, 94%, 95%, 96%, 97%, 98%, 99% or 100% sequence identity, to SEQ ID NO: 36. In some embodiments, X at amino acid residue 164 in SEQ ID NO: 36 is any amino acid or an amino acid selected from the group consisting of A, F, G, I, L, M, S, T and V, particularly A. In some embodiments, the G protein-coupled receptor comprising an integrated cpFP sensor comprises an opioid receptor delta 1 (OPRD1) having at least 90% sequence identity, e.g., at least about 91%, 92%, 93%, 94%, 95%, 96%, 97%, 98%, 99% or 100% sequence identity, to SEQ ID NO: 38. In some embodiments, X at amino acid residue 154 in SEQ ID NO: 38 is any amino acid or an amino acid selected from the group consisting of A, F, G, I, L, M, S, T and V, particularly A. In some embodiments, the G protein couple receptor comprising an integrated cpFP sensor comprises a melatonin receptor 1B (MTNR1B) having at least 90% sequence identity, e.g., at least about 91%, 92%, 93%, 94%, 95%, 96%, 97%, 98%, 99% or 100% sequence identity, to SEQ ID NO: 39. In some embodiments, X at amino acid residue 146 in SEQ ID NO: 39 is any amino acid or an amino acid selected from the group consisting of A, F, G, I, L, M, S, T and V, particularly A. In some embodiments, the G protein-coupled receptor comprising an integrated cpFP sensor comprises a cannabinoid receptor type 1 (CNR1) having at least 90% sequence identity, e.g., at least about 91%, 92%, 93%, 94%, 95%, 96%, 97%, 98%, 99% or 100% sequence identity, to SEQ ID NO: 40. In some embodiments, X at amino acid residue 222 in SEQ ID NO: 40 is any amino acid or an amino acid selected from the group consisting of A, F, G, I, L, M, S, T and V, particularly A. In some embodiments, the G protein-coupled receptor comprising an integrated cpFP sensor comprises a histamine receptor H1 (HRH1) having at least 90% sequence identity, e.g., at least about 91%, 92%, 93%, 94%, 95%, 96%, 97%, 98%, 99% or 100% sequence identity, to SEQ ID NO: 41. In some embodiments, X at amino acid residue 133 in SEQ ID NO: 41 is any amino acid or an amino acid selected from the group consisting of A, F, G, I, L, M, S, T and V, particularly A. In some embodiments, the G protein-coupled receptor comprising an integrated cpFP sensor comprises a neuropeptide Y receptor Y1 (NPY1R) having at least 90% sequence identity, e.g., at least about 91%, 92%, 93%, 94%, 95%, 96%, 97%, 98%, 99% or 100% sequence identity, to SEQ ID NO: 42. In some embodiments, the G protein-coupled receptor comprising an integrated cpFP sensor comprises a muscarinic cholinergic receptor type 2 (CHRM2) having at least 90% sequence identity, e.g., at least about 91%, 92%, 93%, 94%, 95%, 96%, 97%, 98%, 99% or 100% sequence identity, to SEQ ID NO: 43. In some embodiments, X at amino acid residue 129 in SEQ ID NO: 43 is any amino acid or an amino acid selected from the group consisting of A, F, G, I, L, M, S, T and V, particularly A. In some embodiments, the G protein-coupled receptor comprising an integrated cpFP sensor comprises a hypocretin (orexin) receptor 1 (HCRTR1) having at least 90% sequence identity, e.g., at least about 91%, 92%, 93%, 94%, 95%, 96%, 97%, 98%, 99% or 100% sequence identity, to SEQ ID NO: 44. In some embodiments, X at amino acid residue 152 in SEQ ID NO: 44 is any amino acid or an amino acid selected from the group consisting of A, F, G, I, L, M, S, T and V, particularly A.

3. Production of Circularly Permuted Fluorescent Protein Sensors and GPCRs with an Integrated cpFP Sensor

Fluorescent protein sensors can be produced in any number of ways, all of which are well known in the art. In one embodiment, the fluorescent protein sensors are generated using recombinant techniques. One of skill in the art can readily determine nucleotide sequences that encode the desired polypeptides using standard methodology and the teachings herein. Oligonucleotide probes can be devised based on the known sequences and used to probe genomic or cDNA libraries. The sequences can then be further isolated using standard techniques and, e.g., restriction enzymes employed to truncate the gene at desired portions of the full-length sequence. Similarly, sequences of interest can be isolated directly from cells and tissues containing the same, using known techniques, such as phenol extraction and the sequence further manipulated to produce the desired truncations. See, e.g., Green and Sambrook, Molecular Cloning: A Laboratory Manual (Fourth Edition), 2012, Cold Spring Harbor Laboratory Press and Ausubel, et al., eds. Current Protocols in Molecular Biology, 1987-2016, John Wiley & Sons (http://onlinelibrary.wiley.com/book/10.1002/0471142727), for a description of techniques used to obtain, isolate and manipulate nucleic acids. In some embodiments, Circular Polymerase Extension Cloning (CPEC) can be used to insert a polynucleotide encoding a cpFP sensor into a polynucleotide encoding a GPCR. See, e.g., Quan, et al., Nat Protoc, 2011. 6(2): p. 242-51.

The sequences encoding polypeptides can also be produced synthetically, for example, based on the known sequences. The nucleotide sequence can be designed with the appropriate codons for the particular amino acid sequence desired. The complete sequence is generally assembled from overlapping oligonucleotides prepared by standard methods and assembled into a complete coding sequence. See, e.g., Edge (1981) Nature 292:756; Nambair et al. (1984) Science 223:1299; Jay et al. (1984) J. Biol. Chem. 259:6311; Stemmer et al. (1995) Gene 164:49-53.

Recombinant techniques are readily used to clone sequences encoding polypeptides useful in the present fluorescent protein sensors that can then be mutagenized in vitro by the replacement of the appropriate base pair(s) to result in the codon for the desired amino acid. Such a change can include as little as one base pair, effecting a change in a single amino acid, or can encompass several base pair changes. Alternatively, the mutations can be effected using a mismatched primer that hybridizes to the parent nucleotide sequence (generally cDNA corresponding to the RNA sequence), at a temperature below the melting temperature of the mismatched duplex. The primer can be made specific by keeping primer length and base composition within relatively narrow limits and by keeping the mutant base centrally located. See, e.g., Innis et al, (1990) PCR Applications: Protocols for Functional Genomics; Zoller and Smith, Methods Enzymol. (1983) 100:468. Primer extension is effected using DNA polymerase, the product cloned and clones containing the mutated DNA, derived by segregation of the primer extended strand, selected. Selection can be accomplished using the mutant primer as a hybridization probe. The technique is also applicable for generating multiple point mutations. See, e.g., Dalbie-McFarland et al. Proc. Natl. Acad. Sci. USA (1982) 79:6409.

Once coding sequences have been isolated and/or synthesized, they can be cloned into any suitable vector or replicon for expression. As will be apparent from the teachings herein, a wide variety of vectors encoding modified polypeptides can be generated by creating expression constructs which operably link, in various combinations, polynucleotides encoding polypeptides having deletions or mutations therein.

Numerous cloning vectors are known to those of skill in the art, and the selection of an appropriate cloning vector is a matter of choice. Examples of recombinant DNA vectors for cloning and host cells which they can transform include the bacteriophage λ (E. coli), pBR322 (E. coli), pACYC177 (E. coli), pKT230 (gram-negative bacteria), pGV1106 (gram-negative bacteria), pLAFRI (gram-negative bacteria), pME290 (non-E. coli gram-negative bacteria), pHV14 (E. coli and Bacillus subtilis), pBD9 (Bacillus), pIJ61 (Streptomyces), pUC6 (Streptomyces), YIp5 (Saccharomyces), YCp19 (Saccharomyces) and bovine papilloma virus (mammalian cells). See, generally, Green and Sambrook, supra; and Ausubel, supra.

Insect cell expression systems, such as baculovirus systems, can also be used and are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif (“MaxBac” kit).

Plant expression systems can also be used to produce the fluorescent protein sensors described herein. Generally, such systems use virus-based vectors to transfect plant cells with heterologous genes. For a description of such systems see, e.g., Porta et al., Mol. Biotech. (1996) 5:209-221; and Hackland et al., Arch. Virol. (1994) 139:1-22.

Viral systems, such as a vaccinia based infection/transfection system, as described in Tomei et al., J. Virol. (1993) 67:4017-4026 and Selby et al., J. Gen. Virol. (1993) 74:1103-1113, will also find use. In this system, cells are first transfected in vitro with a vaccinia virus recombinant that encodes the bacteriophage T7 RNA polymerase. This polymerase displays exquisite specificity in that it only transcribes templates bearing T7 promoters. Following infection, cells are transfected with the DNA of interest, driven by a T7 promoter. The polymerase expressed in the cytoplasm from the vaccinia virus recombinant transcribes the transfected DNA into RNA that is then translated into protein by the host translational machinery. The method provides for high level, transient, cytoplasmic production of large quantities of RNA and its translation product(s). Other viral systems that find use include adenovirus, adeno-associated virus, lentivirus and retrovirus.

The gene can be placed under the control of a promoter, ribosome binding site (for bacterial expression) and, optionally, an operator (collectively referred to herein as “control” elements), so that the DNA sequence encoding the desired polypeptide is transcribed into RNA in the host cell transformed by a vector containing this expression construction. The coding sequence may or may not contain a signal peptide or leader sequence. Both the naturally occurring signal peptides and heterologous sequences can be used. Leader sequences can be removed by the host in post-translational processing. See, e.g., U.S. Pat. Nos. 4,431,739; 4,425,437; 4,338,397. Such sequences include, but are not limited to, the TPA leader, as well as the honey bee mellitin signal sequence.

Other regulatory sequences may also be desirable which allow for regulation of expression of the protein sequences relative to the growth of the host cell. Such regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

The control sequences and other regulatory sequences may be ligated to the coding sequence prior to insertion into a vector. Alternatively, the coding sequence can be cloned directly into an expression vector that already contains the control sequences and an appropriate restriction site.

In some cases it may be necessary to modify the coding sequence so that it may be attached to the control sequences with the appropriate orientation; i.e., to maintain the proper reading frame. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis, are well known to those skilled in the art. See, generally, Green and Sambrook, supra; and Ausubel, supra.

The expression vector is then used to transform an appropriate host cell. A number of mammalian cell lines are known in the art and include immortalized cell lines available from the American Type Culture Collection (ATCC), such as, but not limited to, Chinese hamster ovary (CHO) cells, HeLa cells, HEK 293T cells, baby hamster kidney (BHK) cells, monkey kidney cells (COS), human hepatocellular carcinoma cells (e.g., Hep G2), Vero293 cells, as well as others. Similarly, bacterial hosts such as E. coli, Bacillus subtilis, and Streptococcus spp., will find use with the present expression constructs. Yeast hosts useful include inter alia, Saccharomyces cerevisiae, Candida albicans, Candida maltosa, Hansenula polymorphs, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

Depending on the expression system and host selected, the fluorescent protein sensors are produced by growing host cells transformed by an expression vector described above under conditions whereby the protein of interest is expressed. The selection of the appropriate growth conditions is within the skill of the art.

4. Fluorescent Biosensors

In some embodiments, the present invention provides a fluorescent biosensor comprising: a 5-HT2A receptor; and a circularly permuted green fluorescent protein (cpGFP) integrated in the third intracellular loop (IL3) of the 5-HT2A receptor.

In some embodiments, the cpGFP is inserted between Lys263 and Ser316 of the 5-HT2A receptor. In some embodiments, the 5-HT2A comprises the polypeptide LSSGY-cpGFP-MHDQL (SEQ ID NO:49). In some embodiments, the 5-HT2A comprises the polypeptide LSSX1X2-cpGFP-X3X4DQL (SEQ ID NO:51), wherein X1, X2, X3, X4 are independently any amino acid.

In some embodiments, X1X2 is selected from the group consisting of leucine-isoleucine (LI), alanine-valine (AV), isoleucine-lysine (IK), serine-arginine (SR), lysine-valine (KV), leucine-alanine (LA), cysteine-proline (CP), glycine-methionine (GM), valine-arginine (VR), asparagine-valine (NV), arginine-valine (RV), arginine-glycine (RG), leucine-glutamate (LE), serine-glycine (SG), valine-aspartate (VD), alanine-phenylalanine (AF), threonine-aspartate (TD), methionine-arginine (MR), leucine-glycine (LG), arginine-glutamine (RQ), serine-tryptophan (SW), serine-glycine (SG), valine-aspartate (VD), leucine-glutamate (LE), alanine-phenylalanine (AF), serine-tryptophan (SW), arginine-glycine (RG), threonine-aspartate (TD), leucine-glycine (LG), arginine-glutamine (RQ), threonine-tyrosine (TY), leucine-leucine (LL), valine-leucine (VL), threonine-glutamine (TQ), valine-phenylalanine (VF), threonine-threonine (TT), leucine-valine (LV), valine-isoleucine (VI), valine-valine (VV), proline-valine (PV), glycine-valine (GV), serine-valine (SV), phenylalanine-valine (FV), cysteine-valine (CV), glutamate-valine (EV), glutamine-valine (QV), and lysine-valine (KV), arginine-tryptophan (RW), glycine-aspartate (GD), alanine-leucine (AL), proline-methionine (PM), glycine-arginine (GR), glycine-tyrosine (GY), isoleucine-cysteine (IC), and glycine-leucine (GL). In some embodiments, X3X4 is selected from the group consisting of asparagine-histidine (NH), threonine-arginine (TR), isoleucine-isoleucine (II), proline-proline (PP), leucine-phenylalanine (LF), valine-threonine (VT), glutamine-glycine (QG), alanine-leucine (AL), proline-arginine (PR), arginine-glycine (RG), threonine-leucine (TL), threonine-proline (TP), glycine-valine (GV), threonine-threonine (TT), cysteine-cysteine (CC), alanine-threonine (AT), leucine-proline (LP), tyrosine-proline (YP), tryptophan-proline (WP), serine-leucine (SL), glutamate-arginine (ER), methionine-cysteine (MC), methionine-histidine (MH), tryptophan-leucine (YL), leucine-serine (LS), arginine-proline (RP), lysine-proline (KP), tyrosine-proline (YP), tryptophan-proline (WP), serine-serine (SS), glycine-valine (GV), valine-serine (VS), glutamine-asparagine (QN), lysine-serine (KS), lysine-threonine (KT), lysine-histidine (KH), lysine-valine (KV), lysine-glutamine (KQ), lysine-arginine (KR), cysteine-proline (CP), alanine-proline (AP), serine-proline (SP), isoleucine-proline (IP), tyrosine-proline (YP), threonine-proline (TP), arginine-proline (RP), aspartate-histidine (DH), histidine-tyrosine (HY), glycine-glycine (GG), proline-histidine (PH), serine-threonine (ST), arginine-serine (RS), arginine-histidine (RH), and tryptophan-proline (WP). In some embodiments, X1X2 comprises alanine-valine (AV) and X3X4 comprises lysine-proline (KP); threonine-arginine (TR); aspartate-histidine (DH); threonine-threonine (TT); serine-serine (SS); glycine-valine (GV); cysteine-cysteine (CC); valine-serine (VS); glutamine-asparagine (QN); lysine-serine (KS); lysine-threonine (KT); lysine-histidine (KH); lysine-valine (KV); lysine-glutamine (KQ); lysine-arginine (KR); lysine-proline (KP); cysteine-proline (CP); alanine-proline (AP); serine-proline (SP); isoleucine-proline (IP); tyrosine-proline (YP); threonine-proline (TP); or arginine-proline (RP); X1X2 comprises leucine-valine (LV) and X3X4 comprises threonine-arginine (TR), lysine-proline (KP) or valine-threonine (VT); X1X2 comprises arginine-valine (RV) and X3X4 comprises threonine-arginine (TR), lysine-proline (KP) or threonine-proline (TP); X1X2 comprises arginine-glycine (RG) and X3X4 comprises tyrosine-leucine (YL) or threonine-arginine (TR); X1X2 comprises serine-arginine (SR) and X3X4 comprises leucine-phenylalanine (LF) or proline-proline (PP); X1X2 comprises proline-methionine (PM) and X3X4 comprises proline-histidine (PH) or serine-serine (SS); X1X2 comprises valine-valine (VV) and X3X4 comprises threonine-arginine (TR) or lysine-proline (KP); XlX2 comprises leucine-isoleucine (LI) and X3X4 comprises threonine-arginine (TR); X1X2 comprises threonine-tyrosine (TY) and X3X4 comprises threonine-arginine (TR); X1X2 comprises isoleucine-lysine (IK) and X3X4 comprises isoleucine-isoleucine (II); X1X2 comprises cysteine-proline (CP) and X3X4 comprises alanine-leucine (AL); X1X2 comprises glycine-methionine (GM) and X3X4 comprises proline-arginine (PR); X1X2 comprises leucine-alanine (LA) and X3X4 comprises glutamine-glycine (QG); X1X2 comprises valine-arginine (VR) and X3X4 comprises arginine-glycine (RG); X1X2 comprises serine-glycine (SG) and X3X4 comprises tyrosine-proline (YP); X1X2 comprises valine-aspartate (VD) and X3X4 comprises tryptophan-proline (WP); X1X2 comprises leucine-glutamate (LE) and X3X4 comprises leucine-proline (LP); X1X2 comprises alanine-phenylalanine (AF) and X3X4 comprises serine-leucine (SL); X1X2 comprises serine-tryptophan (SW) and X3X4 comprises arginine-proline (RP); X1X2 comprises threonine-aspartate (TD) and X3X4 comprises glutamate-arginine (ER); X1X2 comprises leucine-glycine (LG) and X3X4 comprises methionine-histidine (MH); X1X2 comprises arginine-glutamine (RQ) and X3X4 comprises leucine-serine (LS); X1X2 comprises methionine-arginine (MR) and X3X4 comprises methionine-cysteine (MC); X1X2 comprises leucine-leucine (LL) and X3X4 comprises threonine-arginine (TR); X1X2 comprises valine-leucine (VL) and X3X4 comprises threonine-arginine (TR); X1X2 comprises threonine-glutamine (TQ) and X3X4 comprises threonine-arginine (TR); X1X2 comprises valine-phenylalanine (VF) and X3X4 comprises threonine-arginine (TR); X1X2 comprises threonine-threonine (TT) and X3X4 comprises threonine-arginine (TR); X1X2 comprises valine-isoleucine (VI) and X3X4 comprises threonine-arginine (TR); X1X2 comprises proline-valine (PV) and X3X4 comprises lysine-proline (KP); X1X2 comprises glycine-valine (GV) and X3X4 comprises lysine-proline (KP); X1X2 comprises serine-valine (SV) and X3X4 comprises lysine-proline (KP); X1X2 comprises asparagine-valine (NV) and X3X4 comprises lysine-proline (KP); X1X2 comprises phenylalanine-valine (FV) and X3X4 comprises lysine-proline (KP); X1X2 comprises cysteine-valine (CV) and X3X4 comprises lysine-proline (KP); X1X2 comprises glutamate-valine (EV) and X3X4 comprises lysine-proline (KP); X1X2 comprises glutamine-valine (QV) and X3X4 comprises lysine-proline (KP); X1X2 comprises lysine-valine (KV) and X3X4 comprises lysine-proline (KP); X1X2 comprises arginine-tryptophan (RW) and X3X4 comprises histidine-tyrosine (HY); X1X2 comprises glycine-aspartate (GD) and X3X4 comprises glycine-glycine (GG); X1X2 comprises alanine-leucine (AL) and X3X4 comprises asparagine-histidine (NH); X1X2 comprises glycine-arginine (GR) and X3X4 comprises serine-threonine (ST); X1X2 comprises glycine-tyrosine (GY) and X3X4 comprises arginine-serine (RS); X1X2 comprises isoleucine-cysteine (IC) and X3X4 comprises arginine-histidine (RH); or X1X2 comprises glycine-leucine (GL) and X3X4 comprises tryptophan-proline (WP). In some embodiments, X1 is L, X2 is I, X3 is N and X4 is H. In some embodiments, X1 is G, X2 is Y, X3 is M, and X4 is H.

In some embodiments, the 5-HT2A comprises the polypeptide LSSLI-cpGFP-NHDQL (SEQ ID NO:50). In some embodiments, the polypeptide LSSGY-cpGFP-MHDQL (SEQ ID NO:49) is inserted between Lys²⁶³ and Ser³¹⁶ of the 5-HT2A receptor. In some embodiments, the 5-HT2A receptor comprises a transmembrane helix 5 (TM5) comprising a point mutation E264Q. In some embodiments, the 5-HT2A receptor comprises transmembrane helix 6 (TM6) comprising a deletion of Ser³¹⁶. In some embodiments, the 5-HT2A receptor comprises intracellular loop 2 (ICL2) comprising a point mutation Ile^{181A}. In some embodiments, the fluorescent biosensor comprises: the 5-HT2A receptor; the polypeptide LSSGY-cpGFP-MHDQL (SEQ ID NO:49) inserted between Lys²⁶³ and Ser³¹⁶ of the 5-HT2A receptor; the transmembrane helix 5 (TM5) of the 5-HT2A receptor comprises the point mutation E264Q; the transmembrane helix 6 (TM6) of the 5-HT2A receptor comprises the deletion of Ser³¹⁶; and the intracellular loop 2 (ICL2) of the 5-HT2A receptor comprises the point mutation Ile^{181A}. In some embodiments, the fluorescent biosensor comprises the sequence of SEQ ID NO:52.

In some embodiments, the fluorescent biosensor further comprises an ER export peptide on the C-terminus. In some embodiments, the present invention provides a fluorescent biosensor comprising: a 5-HT2A receptor; a circularly permuted green fluorescent protein (cpGFP) integrated in the third intracellular loop (IL3) of the 5-HT2A receptor; and an ER export peptide on the C-terminus. In some embodiments, the ER export peptide is FCYENEV.

In some embodiments, the fluorescent biosensor comprises: a 5-HT2A receptor; a circularly permuted green fluorescent protein (cpGFP) inserted between Lys263 and Ser316 of the 5-HT2A receptor; and an ER export peptide on the C-terminus, wherein the ER export peptide is FCYENEV.

In some embodiments, the fluorescent biosensor comprises: the 5-HT2A receptor; the polypeptide LSSGY-cpGFP-MHDQL (SEQ ID NO:49) inserted between Lys²⁶³ and Ser³¹⁶ of the 5-HT2A receptor; the transmembrane helix 5 (TM5) of the 5-HT2A receptor comprises the point mutation E264Q; the transmembrane helix 6 (TM6) of the 5-HT2A receptor comprises the deletion of Ser³¹⁶; the intracellular loop 2 (ICL2) of the 5-HT2A receptor comprises the point mutation Ile^{181A}; and an ER export peptide on the C-terminus of the fluorescent biosensor, wherein the ER export peptide is FCYENEV. In some embodiments, the fluorescent biosensor comprises the sequence of SEQ ID NO:53.

### IV. Methods

In some embodiments, the present invention provides a method of detecting a ligand-induced hallucinogenic conformational change of a G Protein-Coupled Receptor (GPCR), the method comprising: contacting the ligand with a fluorescent biosensor under conditions for the ligand to bind to the fluorescent biosensor, wherein the fluorescent biosensor comprises the GPCR, and a circularly permuted green fluorescent protein (cpGFP) integrated into the third intracellular loop (IL3) of the GPCR; and measuring the change in fluorescence of the biosensor, thereby detecting the conformational change.

In some embodiments, the GPCR is a 5-HT receptor. In some embodiments, the GPCR is a 5-HT2A receptor. In some embodiments, the cpGFP is inserted between Lys263 and Ser316 of the 5-HT2A receptor.

In some embodiments, the 5-HT2A comprises the polypeptide LSSGY-cpGFP-MHDQL (SEQ ID NO:49). In some embodiments, the polypeptide LSSGY-cpGFP-MHDQL (SEQ ID NO:49) is inserted between Lys263 and Ser316 of the 5-HT2A receptor. In some embodiments, the transmembrane helix 5 (TM5) comprises the point mutation E264Q. In some embodiments, the transmembrane helix 6 (TM6) comprises the deletion of Ser316. In some embodiments, the intracellular loop 2 (ICL2) comprises the point mutation Ile181A. In some embodiments, the cpGFP comprises GCaMP6.

In some embodiments, the fluorescent biosensor further comprises an ER export peptide on the C-terminus. In some embodiments, the ER export peptide is FCYENEV.

In some embodiments, the present invention provides a method of detecting a hallucinogenic compound, the method comprising: contacting a compound with a fluorescent biosensor under conditions for the compound to bind to the fluorescent biosensor, wherein the fluorescent biosensor comprises a G Protein-Coupled Receptor (GPCR), and a circularly permuted green fluorescent protein (cpGFP) integrated into the third intracellular loop (IL3) of the GPCR; and measuring the change in fluorescence of the biosensor, wherein an increase in fluorescence indicates the presence of the hallucinogenic compound, thereby detecting the hallucinogenic compound.

In some embodiments, the GPCR is a 5-HT receptor. In some embodiments, the GPCR is a 5-HT2A receptor. In some embodiments, the cpGFP is inserted between Lys263 and Ser316 of the 5-HT2A receptor.

In some embodiments, the 5-HT2A comprises the polypeptide LSSGY-cpGFP-MHDQL (SEQ ID NO:49). In some embodiments, the polypeptide LSSGY-cpGFP-MHDQL (SEQ ID NO:49) is inserted between Lys²⁶³ and Ser³¹⁶ of the 5-HT2A receptor. In some embodiments, the transmembrane helix 5 (TM5) comprises the point mutation E264Q. In some embodiments, the transmembrane helix 6 (TM6) comprises the deletion of Ser316. In some embodiments, the intracellular loop 2 (ICL2) comprises the point mutation Ile181A. In some embodiments, the cpGFP comprises GCaMP6.

In some embodiments, the fluorescent biosensor further comprises an ER export peptide on the C-terminus.

In some embodiments, the ER export peptide is FCYENEV.

In some embodiments, the present invention provides a method of detecting a non-hallucinogenic antidepressant compound, the method comprising: contacting a compound with a fluorescent biosensor under conditions for the compound to bind to the fluorescent biosensor, wherein the fluorescent biosensor comprises a G Protein-Coupled Receptor (GPCR), and a circularly permuted green fluorescent protein (cpGFP) integrated into the third intracellular loop (IL3) of the GPCR; and measuring the change in fluorescence of the biosensor, wherein a decrease in fluorescence indicates the presence of the non-hallucinogenic antidepressant compound, thereby detecting the non-hallucinogenic antidepressant compound.

In some embodiments, the GPCR is a 5-HT receptor. In some embodiments, the GPCR is a 5-HT2A receptor. In some embodiments, the cpGFP is inserted between Lys²⁶³ and Ser³¹⁶ of the 5-HT2A receptor.

In some embodiments, the 5-HT2A comprises the polypeptide LSSGY-cpGFP-MHDQL (SEQ ID NO:49). In some embodiments, the polypeptide LSSGY-cpGFP-MHDQL (SEQ ID NO:49) is inserted between Lys²⁶³ and Ser³¹⁶ of the 5-HT2A receptor. In some embodiments, the transmembrane helix 5 (TM5) comprises the point mutation E264Q. In some embodiments, the transmembrane helix 6 (TM6) comprises the deletion of Ser³¹⁶. In some embodiments, the intracellular loop 2 (ICL2) comprises the point mutation Ile^{181A}. In some embodiments, the cpGFP comprises GCaMP6.

In some embodiments, the fluorescent biosensor further comprises an ER export peptide on the C-terminus. In some embodiments, the ER export peptide is FCYENEV.

In some embodiments, the present invention provides a method of measuring the hallucinogenic potential of a compound, comprising contacting the compound with a fluorescent biosensor of the present invention, and measuring the agonist effect of the compound on the fluorescent biosensor.

In some embodiments, the present invention provides a method of measuring the antipsychotic potential of a compound, comprising contacting the compound with a fluorescent biosensor of the present invention, and measuring the agonist or antagonist effect of the compound on the fluorescent biosensor.

In some embodiments, the present invention provides a method of identifying a hallucinogenic compound from a non-hallucinogenic compound, the method comprising: contacting a compound with a fluorescent biosensor under agonist conditions and measuring a first fluorescence signal of the compound, wherein an increase in the first fluoresence signal compared to a first control indicates the compound is hallucinogenic; contacting the compound with the fluorescent biosensor under antagonist conditions and measuring a second fluoresence signal of the compound, wherein a decreased second fluoresence signal compared to a second control indicates the compound is non-hallucinogenic; and combining the first fluoresence signal and the second fluorescence signal to calculate a ligand score where a positive ligand score identifies the compound as a hallucinogenic compound and a negative ligand score identifies the compound as a non-hallucinogenic compound.

In some embodiments, the present invention provides a kit comprising a fluorescent biosensor of the present invention.

In some embodiments, the present invention provides a cell comprising a fluorescent biosensor of the present invention.

### V. Examples

### Example 1. psychLight1 and psychLight2

Results

Development of psychLight

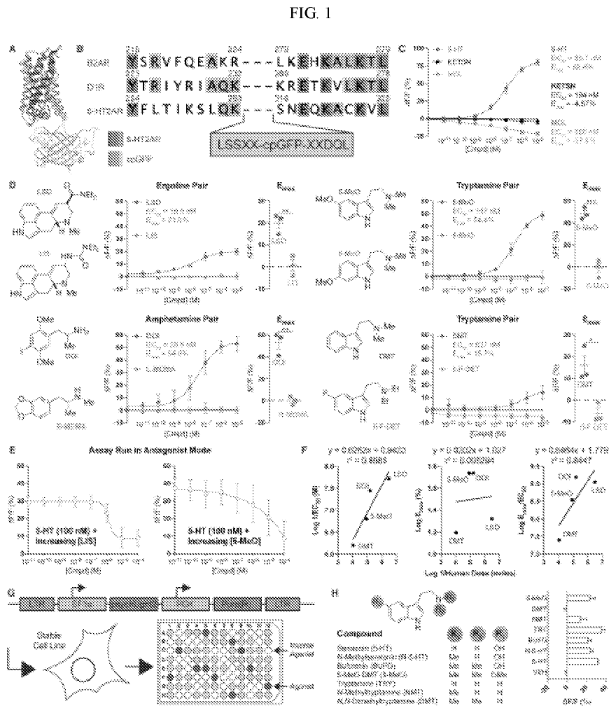

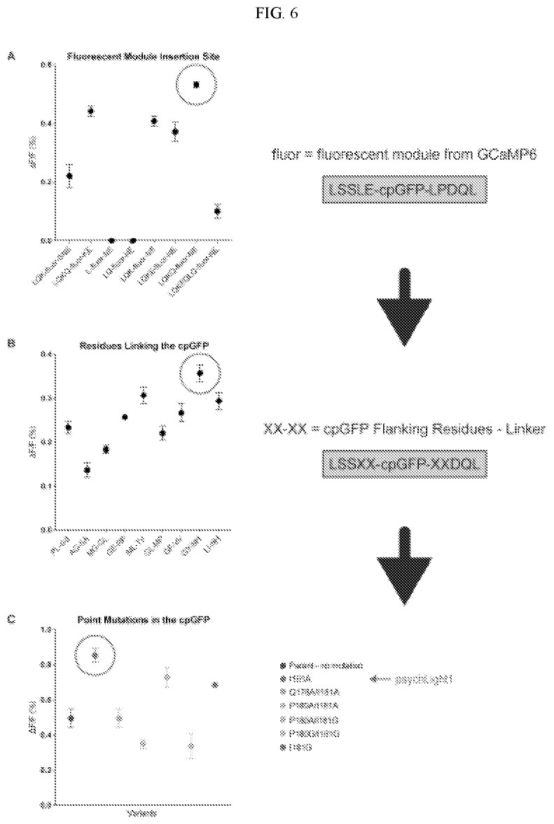

To develop a sensor for the hallucinogenic conformations of the human 5-HT2AR, we envisioned coupling ligand-induced conformational changes to variations in the fluorescence of a circularly permuted green fluorescent protein (cpGFP). A similar modular design was utilized to develop dLight1—a genetically encoded dopamine sensor modeled on the structure of the D1 receptor (D1R). To determine the cpGFP insertion locus most likely to translate ligand-induced conformational changes into large changes in fluorescence intensity, we took advantage of the fact that both the active and inactive structures of the β₂ adrenergic receptor (B2AR) are known and that dLight produces robust changes in fluorescence. After aligning the 5-HT2A receptor with beta-2 adrenergic receptor (B2AR) and the DIR, we decided to replace the third intracellular loop (IL3) with a cpGFP inserted between Lys²⁶³ and Ser³¹⁶ (FIG. 1A and FIG. 1B). The precise insertion site of the cpGFP and the flanking residues were systematically screened to maximize dynamic range in response to 5-HT (FIG. 6). We named the top performing variant psychLight1.

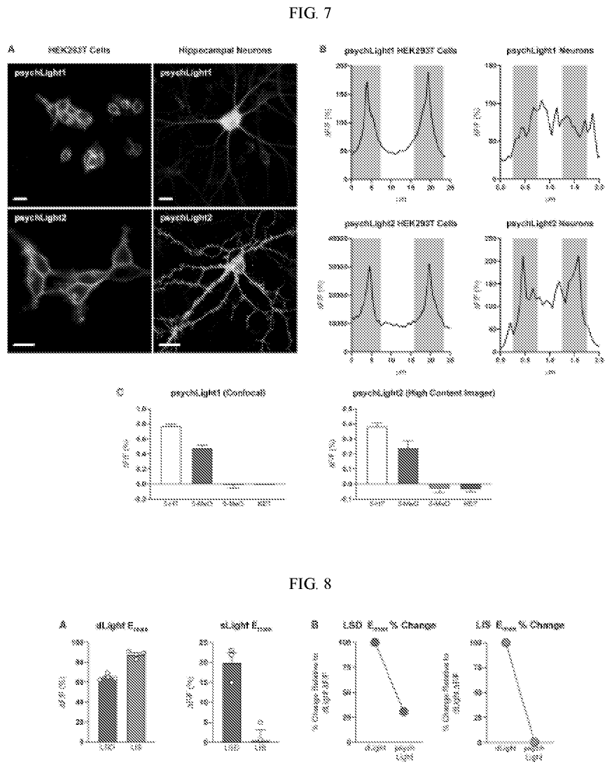

Concentration-response experiments performed with HEK293T cells transiently expressing psychLight1 revealed that 5-HT activates the sensor with an EC₅₀ value of 86.7 nM, which is comparable to values obtained using assays designed to measure G protein and β-arrestin activation (FIG. 1C). The sensor exhibits constitutive activity with unstimulated cells exhibiting low levels of fluorescence (FIG. 7). This property of the sensor enables neutral antagonists (e.g., KETSN) to be distinguished from inverse agonists (e.g., MDL100907) (FIG. 1C) and suggests that psychLight directly reflects specific ligand-dependent conformations of the 5-HT2AR.

When expressed in HEK293T cells, psychLight1 was efficiently trafficked to the plasma membrane; however, the sensor remained primarily intracellular when expressed in dissociated embryonic rat hippocampal neurons (FIG. 7). Fusion of an ER export motif (FCYENEV) to the C-terminus yielded psychLight2, which effectively trafficked to the cell surface, labeling both dendritic shafts and spines (FIG. 7). Control studies revealed that psychLight1 and psychLight2 performed comparably when expressed in HEK293T cells (FIG. 7).

Activation of psychLight Predicts Hallucinogenic Potential

Having established that psychLight responds to 5-HT, but not to antagonists, we next assessed the sensor's ability to differentiate between functionally selective agonists. Previously, Gonzalez-Maeso and co-workers demonstrated that non-hallucinogenic 5-HT2A ligands activate G_{q} through 5-HT2A monomers, while hallucinogenic 5-HT2A ligands can activate G_{i/o} through 5-HT2A-mGluR2 heterodimers, presumably by inducing distinct conformational states. We therefore tested several pairs of hallucinogenic and non-hallucinogenic congeners representing the ergoline, tryptamine, and amphetamine classes of compounds. The propensity of these drugs for causing hallucinations in humans were either known, or inferred from data using well-established rodent models of 5-HT2AR-induced hallucinations such as rat drug discrimination (DD) or mouse head-twitch response (HTR) assays. Results from both DD and HTR studies have been shown to correlate exceptionally well with hallucinogenic potency in humans.

All four hallucinogenic compounds activated psychLight2 with EC₅₀s ranging from 18.8-627 nM. In sharp contrast, none of the non-hallucinogenic congeners were able to activate the sensor, even at concentrations as high as 10 μM (FIG. 1D). The large Eₘₐₓ differences between the hallucinogenic and non-hallucinogenic compounds within a given pair are remarkable given the extremely high degree of structural similarity between the paired molecules (FIG. 1D). Notably, while LSD and LIS displayed functional selectivity for activating psychLight, they both activated dLight to the same extent (FIG. 8).

Next, we attempted to compete off 5-HT (100 nM) with increasing concentrations of non-hallucinogenic compounds. By running the assay in antagonist mode, we were able to demonstrate that non-hallucinogenic compounds like lisuride (LIS) and 6-MeO-DMT (6-MeO) are capable of binding to the receptor despite the fact that they lack efficacy (FIG. 1E). Finally, we found that EC₅₀s for activating psychLight2, but not Eₘₐₓ values, correlated well with human hallucinogenic potencies (r²=0.86, FIG. 1F). The strong correlation is surprising considering the error associated with estimating hallucinogenic potencies in humans and the fact that our cellular assay does not account for potential differences in pharmacokinetics.

Development of a psychLight-Based High-Throughput Screening Assay

Our initial studies indicated that psychLight could be a powerful tool for the high-throughput identification of hallucinogenic designer drugs of abuse as well as non-hallucinogenic medicines targeting 5-HT2A receptors. To achieve this goal, we first generated a cell line stably expressing psychLight2 under the EF1α promoter (FIG. 1G). Using this cell line (PSYLI2) grown in a 96-well plate format, we then confirmed that a widefield high content imager was capable of measuring serotonin-induced changes in psychLight conformations (FIG. 1H). The sensor can detect subtle differences in ligand structure, as it responds to tryptamine-based trace amines to varying degrees (FIG. 1H). Increasing methylation of the basic nitrogen tends to reduce the maximal efficacy (Eₘₐₓ) of the sensor, and a similar structure-activity relationship was previously reported for 5-HT2AR-induced accumulation of [³H]inositol phosphates. Surprisingly, the hydroxyl substituent of 5-HT does not appear to be necessary for achieving full agonism (e.g., 5-HT vs TRY), but it can enhance the activity of partial agonists (e.g., NMT vs N-Me-5-HT, or DMT vs bufotenin) (FIG. 1H). While it has been previously assumed that tryptamine-based trace amines are produced in minute quantities, a recent report suggests that their concentrations in the cortex might be substantially higher than originally thought.

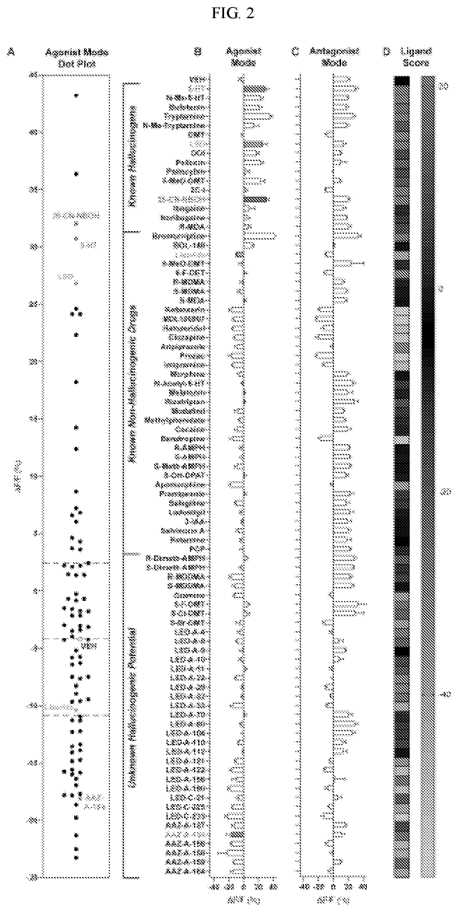

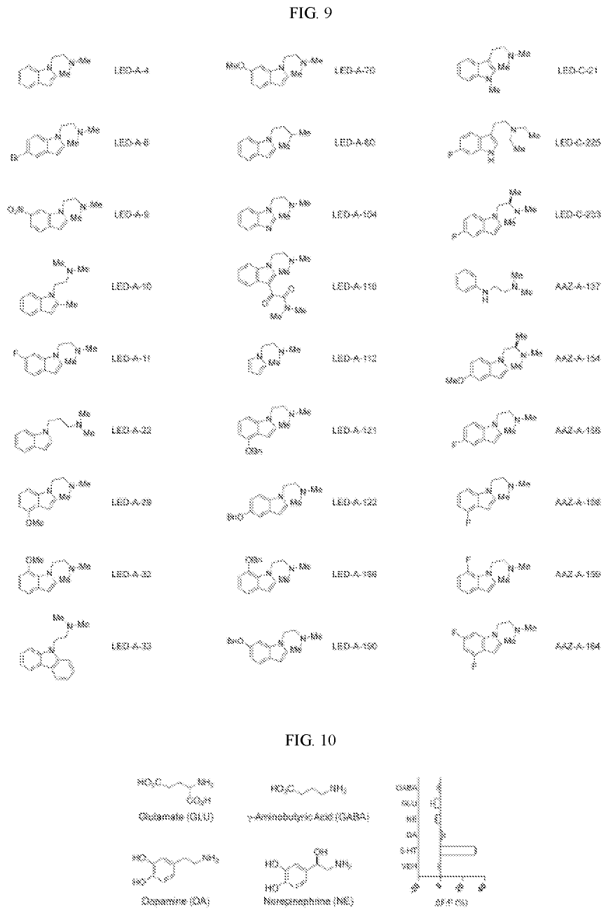

Next, we screened an 87-compound library consisting of known hallucinogens (as defined by human data or predicted based on mouse HTR and/or or DD assays), known non-hallucinogenic 5-HT2A ligands, psychoactive drugs with unknown 5-HT2AR affinity, and novel compounds from our medicinal chemistry program (FIG. 9). When a hit limit of one standard deviation from the vehicle control was applied, the assay was able to reliably differentiate 5-HT2AR-activating hallucinogens from non-hallucinogens FIGS. 2A and B). When the assay was performed in antagonist mode (100 nM 5-HT+10 μM test compound), we were able to identify non-hallucinogenic ligands of the 5-HT2AR (FIG. 2C). To simplify analysis, we defined a “ligand score” (see methods for details) where positive and negative values indicate likely hallucinogenic and non-hallucinogenic ligands of the 5-HT2AR, respectively, while values close to zero indicate compounds that are unlikely to be ligands for the 5-HT2AR (FIG. 2D). The ligand score is particularly useful for identifying compounds that are not likely to be 5-HT2AR ligands.

Of the 16 known serotonergic hallucinogens tested using PSYLI2 cells, DMT was the only false negative (FIGS. 2A and B). Additionally, 35 known non-hallucinogenic psychoactive compounds were screened with only bromocriptine and BOL-148 producing positive signals. While bromocriptine and BOL-148 are both generally considered to be non-hallucinogenic, their lack of hallucinogenic effects could be due to their pharmacokinetics rather than their intrinsic efficacies, especially considering that hallucinations are among the most common side effects reported when these drugs are administered to humans. Finally, non-serotonergic hallucinogens/dissociatives such as salvinorin A, ketamine, and PCP did not activate the sensor FIG. 2C), demonstrating the specificity of this assay for detecting serotonergic hallucinogens.

Identification of Designer Hallucinogenic Drugs Using psychLight

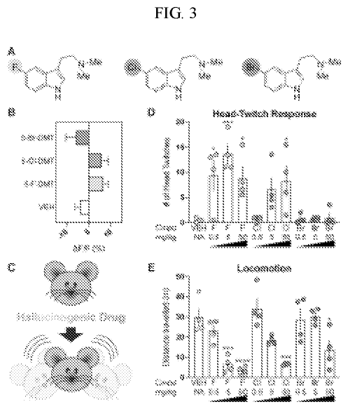

Having validated the potential for psychLight to predict known hallucinogenic and non-hallucinogenic ligands using high-content imaging, we decided to expand the scope of our screen to include 35 compounds synthesized in house with unknown hallucinogenic potential. We identified several potential hallucinogenic hits including R-dimethamphetamine (FIG. 2), but the 5-halo-DMT series really caught our attention due to the high degree of structural similarity between the compounds (FIG. 3A). These compounds only differ by the relative Van der Waals radii of their halogens, yet our psychLight assay predicted that the smaller 5-F-DMT and 5-Cl-DMT would be hallucinogenic, while the larger 5-Br-DMT would not (FIG. 3B). To confirm this prediction in vivo, we performed a three-point dose-response study measuring HTR (FIG. 3C). As expected, both 5-F-DMT and 5-Cl-DMT produced robust HTRs, while 5-Br-DMT did not induce head-twitches at any dose (FIG. 3D). Interestingly, the effects of the compounds on locomotion and did not correlate with HTR, as all drugs at 50 mg/kg significantly reduced locomotion (FIG. 3E).

Identification of a Non-Hallucinogenic Antidepressant Using psychLight

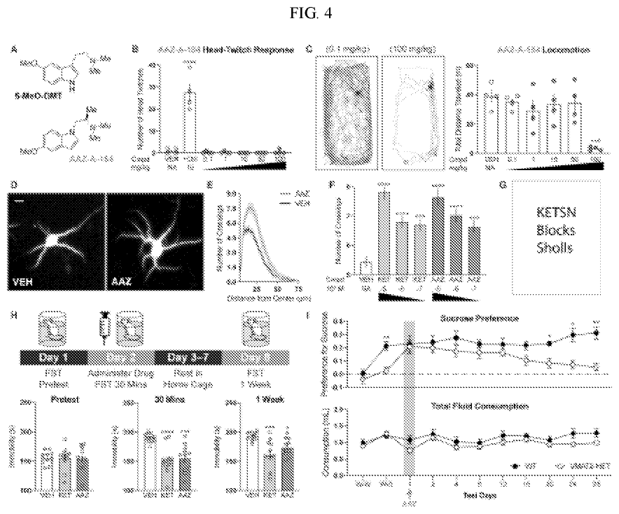

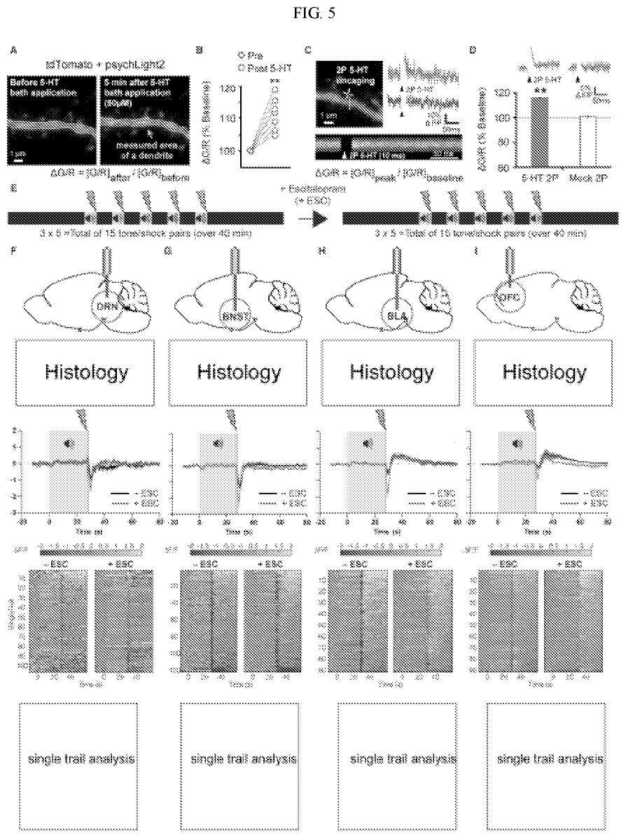

Recent reports have indicated that non-hallucinogenic 5-HT2AR ligands can promote structural neural plasticity in a 5-HT2A-dependent manner. As hallucinogenic and dissociative psychoplastogens are known to produce antidepressant effects, we attempted to use psychLigtht to identify a non-hallucinogenic psychoplastogen with therapeutic properties. We focused our attention on compounds that decreased the intrinsic fluorescence of psychLight (FIGS. 2C and D), and AAZ-A-154 stood out due to its structural similarity to the known hallucinogenic psychoplastogen 5-MeO-DMT (FIG. 4A). We first assessed the hallucinogenic potential of AAZ-A-154 by performing HTR experiments in mice. As expected, AAZ-A-154 did not produce any head-twitches, even up to doses as high as 100 mg/kg (FIG. 4B). However, a high dose of the compound did decrease locomotion (FIG. 4C), indicating that it can still impact behavior without producing hallucinogenic effects.

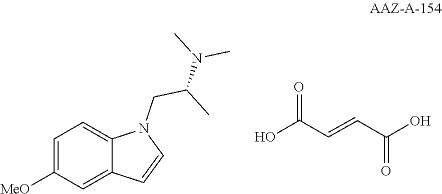 [SMILES: COc1ccc2c(ccn2C[C@@H](C)N(C)C)c1 ; O=C(O)/C=C/C(=O)O]

To assess the antidepressant properties of AAZ-A-154, we first tested its ability to promote the growth of embryonic rat cortical neurons in culture, which is a cellular correlate of antidepressant potential. Treatment with AAZ-A-154 increased dendritic arbor complexity as measured via Sholl analysis to a comparable extent as the state-of-the-art fast-acting antidepressant ketamine, (FIG. 4D-F), and this effect was abrogated by the 5-HT2A antagonist ketanserin (FIG. 4G). As pyramidal neurons in the PFC regulate motivation and reward, changes to the structure/function of these neurons have the potential to produce long-lasting antidepressant effects.

Next, we subjected AAZ-A-154-treated mice to behavioral tests directly relevant to antidepressant potential. First, AAZ-A-154 decreased immobility in the forced swim test (FST) (FIG. 4H)—an effortful behavioral response common to other known psychoplastogens and antidepressants such as ketamine. In these studies, we utilized C57BL/6J mice, as this strain does not respond to traditional antidepressants such as selective serotonin reuptake inhibitors (SSRIs) or tricyclics, thus highlighting the similarity between AAZ-A-154 and next-generation antidepressants like ketamine. Moreover, AAZ-A-154 produces both rapid (30 min) and long-lasting (1 week) antidepressant-like effects after a single administration (FIG. 4H).

To determine if AAZ-A-154 could ameliorate anhedonia, we utilized Vmat2 heterozygous (VMAT2-HET) mice. This genetic model of depression was created because pharmacological inhibition of VMAT2 precipitates depressive-like behaviors, and VMAT2-HET mice display several depressive phenotypes including reduced preference for a 1% sucrose solution over water alone. At baseline, the preference of wild type (WT) animals for sucrose was statistically different than that of VMAT2-HET mice, with WT animals displaying a strong preference for the sucrose solution (FIG. 4I). Immediately following a single administration of AAZ-A-154, the VMAT2-HET mice exhibited a sucrose preference that was indistinguishable from WT controls. This anti-anhedonic effect persisted for at least 16 days before the genotypes began to once again display differences in sucrose preference (FIG. 4I). Importantly, the change in sucrose preference observed for the VMAT2-HET mice cannot be attributed to differential fluid consumption since both genotypes drank similar volumes of liquids across the entire experiment (FIG. 4I). Moreover, the effects of AAZ-A-154 cannot be ascribed to increasing sucrose palatability, as AAZ-A-154 had no effect on WT animals (FIG. 4I).

In Vivo Imaging of Serotonin Dynamics Using psychLight

As psychLight responds robustly to 5-HT, the endogenous ligand for the 5-HT2AR, we were interested in determining if psychLight could be used to probe serotonin transients in vivo. While GPCRs are relatively selective for their endogenous ligands, we first confirmed that psychLight2 exhibited high selectivity for 5-HT. As anticipated, HEK293T cells expressing psychLight do not respond to endogenous neurotransmitters (glutamate, GABA) or monoamines (dopamine, norepinephrine) (FIG. 10). Next, we examined the kinetics of psychLight using two-photon photolysis of RuBi-5-HT in cultured hippocampal? slices. Co-expression of psychLight and tdTomato enable us to normalize for expression level. Bath application of 5-HT (50 μM) led to a significant increase in the ratio of green to red fluorescence intensities measured at nm (psychLight2, G=green) and nm (tdTomato, R=red) (FIGS. 5A and B). Site-specific two-photon photolysis of RuBi-5-HT revealed a rapid (ms) increase in psychLight fluorescence that returned to baseline after XX ms (Tau on and Tau offt FIGS. 5C and D). No increase in fluorescence was observed in cells that expressed GFP lacking the sensing module.

Next, we asked whether psychLight could reliably report serotonin dynamics in awake freely behaving mice. We injected adeno-associated virus encoding psychLight driven by the synapsin promoter (AAV9.hSynapsin.psychLight2 or AAV8.hSynapsin.psychLight2) into the dorsal raphe nucleus (DRN), the bed nuclei of the stria terminalis (BNST), the basolateral amygdala (BLA), and the orbitofrontal cortex (OFC). Then, we implanted a fiber optic cannula directly above each injection site (FIG. 5F-I). After 2-3 weeks of expression, we used fiber photometry to measure 5-HT transients during an auditory fear conditioning experiment consisting of 15 presentations of a 30 s tone co-terminating with a 1.5 s foot shock (0.5 mA) (FIG. 5E).

In the DRN, we observed a robust increase in fluorescence after the onset of foot shock (FIG. 5F), followed by a sharp drop during the shock. These data are consistent with calcium transients recorded in the DRN using GCaMP during auditory fear conditioning. In the BNST, we observed an immediate decrease in fluorescence following the onset of the foot shock that returned to baseline within 4 s (FIG. 5G). A similar initial drop in fluorescence was observed in the BLA and OFC; however, this initial decrease in sensor activity was followed by a large rise in fluorescence signal 2-4 s after the shock (FIGS. 5H and I). Altogether, these data indicate that psychLigth can be used to measure brain-region specific 5-HT dynamics in freely behaving animals.

Finally, we evaluated the effect of an acutely administrated SSRI on serotonin dynamics in this behavioral experiment. Escitalopram (10 mg/kg) was administrated 30 mins prior to imaging. In all brain regions, escitalopram mitigated the reduction in 5-HT levels observed following foot shock (i.e, increased 5-HT levels) (FIG. 5F-I), presumably through inhibition of serotonin transporters. Moreover, in the OFC, escitalopram delayed the return of elevated 5-HT levels to baseline (FIG. 5I).

### DISCUSSION

The 5-HT2A receptor is a prime example of a GPCR capable of accessing multiple conformationally distinct signaling states. Thus, 5-HT2A receptor ligands have demonstrated broad functional effects including antipsychotic, hallucinogenic, and plasticity-promoting properties (e.g., clozapine, LSD, and tabernanthalog, respectively). However, tools capable of directly assessing specific ligand-induced conformational states of this receptor have been lacking. Here, we report psychLight—the first fluorescent sensor capable of detecting hallucinogenic conformations of the 5-HT2A receptor.

Prior to the advent of psychLight, it was necessary to use in vivo behavioral tests to determine the hallucinogenic potential of novel compounds, with the most common being HTR and DD. Now, hallucinogenic potential can be rapidly assessed using a cellular assay with a simple fluorescence readout, drastically reducing the number of animals used in research. Unlike HTR and DD experiments, this cellular assay is not impacted by differences in pharmacokinetics. Thus, psychLight has the potential to provide a more accurate assessment of the hallucinogenic properties of a particular chemical scaffold. For example, 5-HT is generally considered to be non-hallucinogenic due to the fact that it does not readily cross the blood-brain barrier following systemic administration. However, 5-HT produces robust HTR behavior when administered directly to the brain ventricles. PsychLight accurately predicts the ability of these compounds to induce hallucinogenic conformations of the 5-HT2A receptor without the need for in vivo testing.

The development of a high-throughput cellular method for assessing hallucinogenic potential will greatly facilitate at least two important areas of investigation. First, psychLight will enable the rapid identification of chemical scaffolds likely to give rise to designer hallucinogenic drugs of abuse. In contrast to classic psychedelics, these novel drugs are particularly dangerous because they lack human safety data, and thus, have the potential to lead to serious adverse effects or even death. Early knowledge of their potential for abuse will be critical to identify those that pose serious health risks. Here, we used psychLight to identify 5-F-DMT and 5-Cl-DMT as hallucinogenic compounds with previously unknown potential for abuse.

In addition to enabling the identification of novel designer hallucinogens, psychLight can also be used in drug discovery efforts aimed at developing non-hallucinogenic 5-HT2A ligands (e.g., antipsychotics) or non-hallucinogenic analogs of psychedelics (e.g., ergolines and triptans currently in the clinic for treating Parkinson's disease and migraines, respectively). In the past year, non-hallucinogenic psychoplastogens have emerged as a particularly exciting class of 5-HT2A ligands given the broad implications that neural plasticity-promoting compounds have for treating a variety of brain disorders such as depression, PTSD, and substance use disorder. Furthermore, these unique psychoplastogens do not induce hallucinations—a liability that has plagued the clinical development of classic psychedelics.

Here, we used psychLight to identify AAZ-A-154—a non-hallucinogenic analog of a psychedelic compound that promotes neuronal growth and produces long-lasting (>1 week) beneficial behavioral effects in rodent tests relevant to motivation and anhedonia. Tabernanthalog is the only other known non-hallucinogenic psychoplastogen with antidepressant-like properties, and it appears that AAZ-A-154 is not only more potent than tabemanthalog, it also produces more sustained antidepressant effects.

In addition to using psychLight for drug discovery, we demonstrate that this novel sensor can detect serotonin dynamics with high spatiotemporal precision in vivo. Serotonin is an incredibly important neuromodulator, playing key roles in the regulation of mood, memory, aggression, appetite, and sleep, among its many other functions. Therefore, we anticipate that psychLight and other genetically encoded sensors will prove critical for fully understanding the effects of endogenous serotonin on brain function. Taken together, our work outlines a general strategy for engineering GPCR-based conformational sensors, and we anticipate that similar approaches will be used to design sensors capable of directly assessing GPCR conformations relevant to functionally selective ligands.

Creation of PSYLI2 Cell Line Stably Expressing PsychLight2.

The psychLight2 gene was cloned into a pLVX plasmid with the EF1α promotor. The plasmid was transfected into HEK293T cells together with pCMV_delta8.2 and pCMV_VSV_G in a ratio of 10:7:3 using the Qiagen Effectene Transfection kit. After 14 h of incubation, the medium was exchanged for fresh DMEM. After an additional 48 h of incubation, the lentivirus-containing medium was collected, filtered through a 0.45 μm Durapore low-protein binding filter, concentrated using a Centricon-70 ultra filtration unit at 3,500 g for 50 mins, and frozen by storing at −80° C. Next, confluent HEK293T cells grown in 24-well plates were infected with 20 μL of concentrated lentivirus for 48 h. Puromycin selection was performed as described by Tandon and co-workers. Expression was assessed via fluorescence microscopy, and a single cell was selected for expansion. The new cell line, named PSYLI2, was tested and then frozen in 10% DMSO at −80° C. and then transferred to a liquid nitrogen dewar.

High-Content Imaging Experiments. Glass bottom 96-well plates (P96-1.5H-N, Cellvis) were coated with 50 μg/mL of poly-D-lysine (Sigma, P6407-5MG) and 10 μg/mL laminin (Sigma, L2020) overnight in an incubator (37° C., 5% CO₂). Plates were then washed with dPBS (ThermoFisher, 14190-250) and PSYLI2 cells were suspended in DMEM (Fisher, 11995073) containing 10% FBS (Fisher, 26-140-079) with 5% penicillin-streptomycin (Fisher, 15140-163) and plated at a density of 40,000 cells/well 24 hours prior to each experiment. Immediately prior to an experiment, stock solutions of drugs in DMSO (10 mM) were first diluted 1:100 in imaging media distributed across an empty 96-well plate (treatment plate) in triplicates following a randomized plate map. Imaging media consisted of 1×HBSS (Fisher, 14175103) containing 0.5 M MgCl₂ (Sigma, M8266-1KG) and 0.5 M CaCl₂ (Sigma, C5670-50G). Cells grown in a separate 96-well plate (assay plate) were gently washed (3×) with imaging media, and the wells were filled with an appropriate volume of imaging media for the respective experiment (vide infra).

For agonist mode, 180 pL of imaging media was added to each well of the assay plate. Wells were then imaged on a Lecia DMi8 using Leica Application Suite X (V3.6.0.20104) at 40×(N.A.=0.6) with 5 regions of interest (ROI) taken per well using the default 5 ROI pattern for each well with no bias to location and no overlap of the ROIs (exposure=350 ms, LED power=80%). Next, 20 μL from the treatment plate was transferred to the assay plate for a total 1:1000 dilution of drug (10 μM drug, 0.1% DMSO). As positive, negative, and neutral controls, 5-HT (10 μM), ketanserin (10 μM), and DMSO (0.1%) were used, respectively. All final concentrations of drugs were 10 μM (0.1% DMSO) in agonist mode unless stated otherwise. After 5 min of incubation, the same sites were re-imaged using the same settings.

Once imaging was complete, the images were exported, put into a stack, and analyzed using ImageJ Fiji (ver. 1.51v) by using the rectangle function to draw an ROI around the cell membrane (one cell per image) on both the pre- and posttreatment images. ROI sizes within each plate were kept the same and saved to the ROI manager in ImageJ (ctrl+T). Images were then measured using the “multi measure” function in the ROI manager. Measurements were saved and exported. Analysis of the data was done by taking the average of each individual well's ROIs before and after drugs were added. Then the ΔF/F values for each well were calculated using the following equation:

$$\frac{( average after drug - average before drug )}{average before drug ( baseline )}$$

These values were then averaged to obtain the triplicate average (N=3).

For antagonist mode, 160 μL of imaging media was added to each well of the assay plate. Wells were then imaged on a Lecia DMi8 using Leica Application Suite X (V3.6.0.20104) at 40×(N.A.=0.6) with 5 regions of interest (ROI) taken per well using the default 5 ROI pattern for each well with no bias to location and no overlap of the ROIs (exposure=350 ms, LED power=80%). A 100 μM 5-HT stock solution in DMSO was diluted 1:100 in imaging buffer. Next, 20 μL of this solution was added to the assay plate for a final concentration of 111 nM 5-HT (0.1% DMSO). The same 5 ROIs were imaged after 5 min of incubation. Next, 20 μL from the treatment plate was transferred to the assay plate for a total 1:1000 dilution of drug (10 μM drug, 100 nM 5-HT, 0.2% DMSO). All final concentrations of drugs were 10 μM (100 nM 5-HT, 0.2% DMSO) in antagonist mode unless stated otherwise. After 5 min of incubation, the same sites were re-imaged using the same settings.

Once imaging was complete, the images were exported, put into a stack, and analyzed using ImageJ Fiji (ver. 1.51v) by using the rectangle function to draw an ROI around the cell membrane (one cell per image) on both the pre- and posttreatment images. ROI sizes within each plate were kept the same and saved to the ROI manager in ImageJ (ctrl+T). Images were then measured using the “multi measure” function in the ROI manager. Measurements were saved and exported. Analysis of the data was done by taking the average of each individual well's ROIs before and after drugs were added. Then the ΔF/F values for each well were calculated using the following equation:

$$\frac{( average after drug - average after 5 ‐ HT drug )}{average after 5 ‐ HT ( baseline )}$$

These values were then averaged to obtain the triplicate average (N=3). All imaging and incubation (both agonist and antagonist mode) were performed at ambient atmosphere and temperature.

Data Availability

The datasets generated as part of this study are available in the Figshare repository, 10.6084/m9.figshare.11634795.

### Example 2. psychLight1 and psvchLight2

Data and Code Availability

The full sequence of psychLight has been deposited in GenBank:MW285156 (psychLight1), GeneBank: MW285157 (psychLight2).

Experimental Model and Subject Details

Animals

All experimental procedures involving animals were approved by the Institutional Animal Care and Use Committee (IACUC) at the University of California, Davis, the University of Colorado School of Medicine, or Duke University, and adhered to principles described in the National Institutes of Health Guide for the Care and Use of Laboratory Animals. The University of California, Davis, the University of Colorado School of Medicine, and Duke University are accredited by the Association for Assessment and Accreditation of Laboratory Animal Care International (AAALAC).

Method Details

### Abbreviations (in Alphabetical Order)

β2AR=beta-2 adrenergic receptor;
(S)-Meth-AMPH=(S)-methamphetamine;
+Ctrl=positive control;
μm=micrometer;
25-CN-NBOH=4-(2-(2-Hydroxybenzylamino)ethyl)-2,5-dimethoxybenzonitrile hydrochloride;
2C-I=2-(4-Iodo-2,5-dimethoxyphenyl) ethan-1-amine;
2p=2-photon;
3-IAA=indole-3-acetic acid;
5-Br-DMT=5-bromo-N,N-dimethyltryptamine;
5-Cl-DMT=5-chloro-N,N-dimethyltryptamine;
5-F-DMT=5-flouo-N,N-dimethyltryptamine;
5-HT=serotonin;
5-HT2AR=serotonin 2A receptor;
5-MeO=5-methoxy-N,N-dimethyltrytamine;
6-F-DET=6-flouro-N,N-diethyltrytamine;
6-MeO=6-methoxy-N,N-dimethyltrypta-mine;
8-OH-DPAT=(+)-8-hydroxy-2-(dipropylamino)tetralin;
AAV=adeno-associated virus;

### AAZ=AAZ-A-154;

aCSF=artificial cerebrospinal fluid;
BLA=basolateral amygdala;
BNST=bed nucleus of the stria terminalis;
BOL-148=2-bromo-lysergic acid di-ethylamide;
BUFO=bufotenin, N,N-dimethyltryptamine;
cpGFP=circularly permuted green fluorescent protein;
CPMD=compound;
DA=dopamine;
D1R=dopamine receptor D1;
DMSO=dimethyl sulfoxide;

### DMT=N,N-dimethyltrytamine;

DOI=2,5-dime-thoxy-4-iodoamphetamine;
DRN=dorsal raphe nucleus;
EC₅₀=half maximal effective concentration;
EF1α=human elongation factor-1 alpha;
Emax=maximum efficacy;
ESC=escitalopram oxalate;
FST=forced swim test;
GABA=_{y}-aminobutyric acid;
GLU=glutamate;
HEK293T=human embryonic kidney 293 cells with SV40 T-antigen;
HTR=head-twitch response;
IL3=third intracellular loop;
KET=ketamine;
KETSN=ketanserin;
LIS=lisuride;
LSD=lysergic acid diethylamide;
LTR=long terminal repeat;

### MDL=MDL 100907;

### N-5-HT=N-methylserotonin;

### N-acetyl-5-HT=N-acetylserotonin;

NA=not available;
NE=norepinephrine;

### NMT=N-methyltryptamine;

OFC=orbitofrontal cortex;

### PCP=Phencyclidine;

PCP=phencyclidine;
PGK=phosphoglycerate ki-nase;
Puro(R)=puromycin resistance;
R-AMPH=(R)-amphetamine;
R-dimeth-AMPH=(R)-dimethamphetamine;
R-MDA=(R)-3,4-methylenedioxyamphetamine;
R-MDDMA=(R)-3,4-methylenedioxydimethylamphetamine;
R-MDMA=(R)-3,4-methylene-dioxy-methamphetamine;
ROI=region of interest;
S-AMPH=(S)-amphetamine;
S-dimeth-AMPH=(S)-dimethamphetamine;
S-MDA=(S)-3,4-methylenedioxyamphetamine;
S-MDDMA=(S)-3,4-methylenedioxydimethylamphetamine;
S-MDMA=(S)-3,4-methylenedioxy-methamphetamine;
SEM=standard error mean;
STD=standard deviation;
TRY=tryptamine;
TTX=tetrodotoxin citrate;

### U50=U50488;

VEH=vehicle;
VMAT2-HET=vesicular monoamine transporter 2 heterozygous;
W-S=water and 1% sucrose solution;
W-W=water and water;
WT=wild-type;

### TBG=Tabernanthalog;

_{A}F/F=change in fluorescence over initial fluorescence.

Compounds

The NIH Drug Supply Program provided lysergic acid diethylamide hemitartrate, psilocin, psilocybin, 2-(4-Iodo-2,5-dimethoxy-phenyl)ethan-1-amine hydrochloride (2C-I), 2-bromo-lysergic acid diethylamide tartrate (BOL-148), ibogaine hydrochloride, noribo-gaine, cocaine hydrochloride, salvinorin A, and phencyclidine hydrochloride (PCP). Other chemicals were purchased from commercial sources such as serotonin hydrochloride (5-HT, Fisher, 50-120-7920), ketanserin (KETSN, ApexBio, 50-190-5332), ketamine hydrochloride (KET, Fagron, 803647), morphine sulfate (Mallinckrodt, Inc., 0406-1521-53), lisuride maleate (LIS, Tocris, 40-5210), bromocriptine mesylate (Tocris, 04-275-0), (±)-2,5-dimethoxy-4-iodoamphetamine hydrochloride (DOI, Cayman, 13885), imipramine hydrochloride (Cayman, 15890), modafinil (Cayman, 15417), (±)-threo-methylphenidate hydrochloride (Cayman, 11639), indole 3-aceitic acid (3-IAA, ACROS, AC12216-0250), gramine (ACROS, AC12018-0100), N-acetylserotonin (ACROS, AC22693-1000), mela-tonin (ACROS, AC12536-2500), tryptamine (TRY, ACROS, AC15798-0050), N-methyltryptamine (NMT, ACROS, AC151751000), MDL 100907 (MDL, Sigma, M3324-5MG), haloperidol (Sigma, H1512), clozapine (Sigma, C6305), aripiprazole (Sigma, SML0935), fluoxetine hydrochloride (Sigma, F132-10MG), rizatriptan benzoate (Sigma, SML0247-10MG), benztropine mesylate (Sigma, SML0847-500MG), (±)-8-hydroxy-2-(dipropylamino)tetralin hydrobromide (8-OH-DPAT, Sigma, H8520-25MG), R-(−)-apomorphine hydrochloride hemihydrate (Sigma, A4393-100MG), pramipexole hydrochloride (Sigma, PHR1598-500MG), selegiline hydrochloride (Sigma, M003-250MG), ladostigil tartrate (Sigma, SML2263-5MG), RuBi-5-HT (Tocris, 3856) escitalopram oxalate (ESC, Tocris, 4796), L-glutamic acid (GLU, Sigma, G1251-500G), _{y}-aminobutyric acid (GABA, A5835-25G), dopamine hydrochloride (DA, Sigma, H8502-25G), and norepinephrine bitartrate (NE, 1468501). For cellular experiments, the VEH is dimethyl sulfoxide (DMSO, ACROS, AC327182500). For in vivo experiments, VEH=USP grade saline (0.9%, VWR, 68099-103). The remaining compounds used in these studies were synthesized in house and judged to be pure based on NMR and UHPLC-MS. Compounds of the DMT, IsoDMT families (LED-A-4-LED-C-21) and Tabemanthalog (TBG) were prepared as described previously. All enantiopure amphetamines (i.e., amphetamine, methamphetamine, dimethamphetamine) and methylenedioxymethamphet-amines (i.e., MDA, MDMA, MDDMA) were prepared using methodology described by Nenajdenko. The key step involved the regioselective ring opening of enantiopure Boc-protected aziridines derived from R- and S-alaninol, respectively. The Boc-protected amphetamines and methylenedioxymethamphetamines were determined to be enantio-merically pure (>99% ee) by chiral HPLC. The methylated amphetamines and methylated methylenedioxyamphetamines were readily prepared using known methods. All amphetamine and methylenedioxyamphet-amine derivatives were prepared as the 1:1 fumarate salts with the exception of R- and S-MDMA, which were prepared as the 2:1 fumarate salts (i.e., hemifumarates). Lastly, N-Me-5-HT, N-Me-5-MeO-tryptamine hemifumarate, 6-fluorodiethyltryptamine (6-F-DET hemifumarate), 5-bromo-DMT hemifumarate, 5-chloro-DMT hemifumarate, 5-fluoro-DMT hemifumarate, and AAZ-A-137 hemifuma-rate were prepared using previously reported methods. Synthetic procedures and characterization data for AAZ-A-154 and LED-C-233 are reported below.

PsychLight Development and Characterization

Development of PsychLight1 and PsychLight2

All constructs were designed using circular polymerase extension cloning (CPEC), restriction cloning, and gBlock gene fragments (Integrated DNA Technologies). Sequences coding for a FLAG epitope were placed at the 5′ end of the construct as previously described. HindIII and NotI cut sites were placed at the 5′- and 3′ ends, respectively, for cloning into pCMV (Addgene) to generate all pCMV constructs. BamHI and HindIII sites were introduced via PCR for final sub-cloning onto pAAV.hSynapsin1 vectors (Addgene). To maximize coupling between conformational changes and chromophore fluorescence, we chose to use a cpGFP module (LSS-LE-cpGFP-LP-DQL) from GCaMP6 for insertion into the human 5-HT2AR using circular polymerase extension cloning (CPEC).

For screening linker variants, we generated linker libraries by first creating an insert DNA carrying a randomized 2 amino acid linker on each side of cpGFP (LSS-xx-cpGFP-xx-DQL). Cloned constructs were amplified and purified with the QIAGEN PCR purification kit prior to NEB® 5-ₐ competent E. coli transformation. Competent cells were plated onto kanamycin-containing agar plates. After allowing for 24-hour of growth at 37° C., single colonies were manually picked and grown overnight as described previously. Plasmids from the colonies were with purified using the QIAGEN miniprep kit. Top variants were sequenced by Genewiz. For conversion of psychLight1 to psychLight2, an ER2 tag was added to the C terminus of the protein, as described previously, and the two original amino acids from the cpGFP sequence (i.e., F511 and N512, numbering based on psy-chLight2) were inserted into the N-terminal side of linker 2 to increase the baseline fluorescence. NEB® stable competent cells were transformed with PAAV_hSynapsin_psychLight2. After growth on an agar plate at 30° C., a single colony was selected. After sequencing confirmed the presence of the psychLight2 gene, the cells were expanded at 30° C. in 100 mL of growth medium (2×YT), and purified with a QIAGEN Endo-free Plasmid Maxi kit and send to the UC DAVIS Virus Packaging Core for virus production. Sequence information for psychLight1 and psychLight2, see Data S1.

Tissue Culture

HEK293T cells were grown in DMEM, supplemented with fetal bovine serum (FBS) and penicillin-streptomycin. Cells were trans-fected with Effectene according to the manufacturer's instructions. Prior to imaging, cells were washed with Hank's Balanced Salt Solution (HBSS) supplemented with 2 mM MgCl₂ and 2 mM CaCl₂). All images were collected in HBSS containing Mg²⁺ and Ca²⁺.

Transient Transfection of PsychLight1

HEK293T cells were plated and transfected concurrently 24 h prior to each experiment using the QIAGEN Effectene Transfection

Reagent Kit According to the Manufacture's Protocol.

Confocal Microscopy Experiments

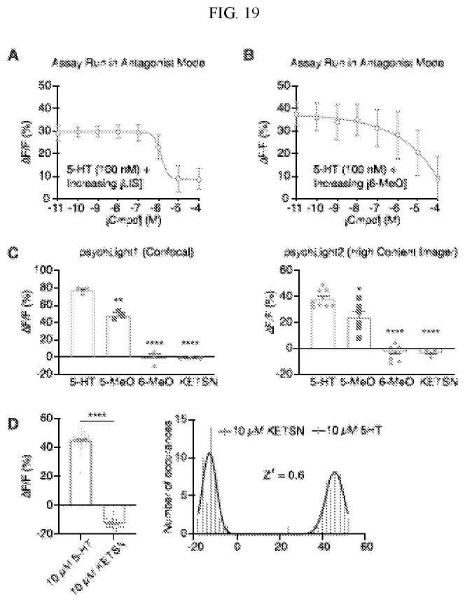

Dose-response experiments were performed using an Automate Perfusion System. Cells (HEK293T) were grown on 12 mm cover-slips and transfected with psychLight1. The coverslips were then placed into a coverslip holder and washed with 5 mL of HBSS containing 2 mM MgCl₂ and 2 mM CaCl₂). Cells were perfused first with 5 mL of 0.1% DMSO, then drugs in ascending concentrations from 1 pM to 10 ₘM were added, with the concentration of DMSO being held constant at 0.10%. Images were recorded using a 465 nm laser and a 40×oil objection (0.55 N.A.) on a Zeiss 710 confocal microscope. For the competition studies described in FIG. 19, HEK293T cells were prepared as described above; however, the cells were first exposed to 5 mL of 0.2% DMSO. Next, 100 nM 5-HT in 0.2% DMSO was introduced to the cells followed by ascending concentrations of the drug (from 1 pM to 10 mM) in a solution of 100 nM 5-HT, with the concentration of DMSO kept constant at 0.2%. Analysis was performed by taking 3 ROIs on the cell membrane using ImageJ and calculating the mean intensity for each ROI across the time-points. Finally, the DF/F was calculated using the average of the baseline (0.1 or 0.2% DMSO) and the average intensity between each dosage over the average of the baseline.

Two-Photon Uncaging Experiments

Organotypic slice cultures from the frontal cortex were prepared from postnatal day 2-3 (P2-P3) C57BL/6J mice, as described previously. Slices were infected 19-20 days prior to imaging by adding a drop of a solution containing 1 tL of concentrated psychLight2 virus (AAV_hSyn_psychLight2) and 4 tL of slice culture media (pre-warmed to 37° C.) to the top of the cortical layers. Slices were transfected with tdTomato 17-18 days prior to imaging using biolistic gene transfer (180 psi). Gold particles (6-7 mg) were coated with 12 tg of the tdTomato plasmid. Two-photon imaging and uncaging were performed after 21-23 days in vitro (DIV) on transfected layer 2/3 pyramidal neurons within 40 tm of the slice surface at 30° C. in recirculating artificial cerebrospinal fluid (aCSF; in mM: 127 NaCl, 25 NaHCO₃, 1.25 NaH₂PO₄, 2.5 KCl, 25 D-glucose, aerated with 95% O₂/5% CO₂) with 2 mM CaCl₂), 1 mM MgCl₂, 0.1 mM RuBi-5-HT, and 0.001 mM tetrodotoxin. For each neuron, image stacks (512 3 512 pixels; 0.047 tm/pixel) with 1 tm z-steps were collected from one segment of secondary or tertiary apical dendrites 50-80 tm from the soma using a two-photon microscope (Bruker) with a pulsed Ti::sapphire laser (Mai Tai, Spectra Physics) tuned to 920 nm (4-5 mW at the sample). All images shown are maximum projections of 3D image stacks after applying a median filter (2 3 2) to the raw image data. Two-photon uncaging was achieved, as previously described, except that RuBi-5-HT was used. In brief, the 5-HT uncaging stimulus (1 pulse of 10-ms duration; 17-20 mW at the sample, 810 nm) was delivered by parking the beam at a point 0.5 tm from the edge of a dendrite with a pulsed Ti::sapphire laser (MaiTai HP, Spectra-Physics). The mock stimulus was identical in parameters to the uncaging stimulus, except carried out in the absence of RuBi-5-HT. Line-scan recording of fluorescence transients was performed simultaneous with 5-HT uncaging on layer 2/3 pyramidal neurons using two pulsed Ti::sap-phire lasers for imaging and uncaging at wavelengths of 920 nm and 810 nm, respectively. The fluorescent measurements of psy-chLight transients were represented as ΔpL2/tdT=[(pL2/tdT) peak/(pL2/tdT) baseline], where pL2 and tdT represent the fluorescence from psychLight2 and tdTomato, respectively. After measuring baseline fluorescences (50 ms), 5-HT uncaging (1 pulse of 10-ms duration, 17-20 mW) was delivered at the target region and peak fluorescences were averaged over 10 ms around the peak. Only cells that showed stable 5-HT-insensitive (Red) signals (<±5% fluctuation) were included in our analysis. To measure changes in psychLight fluorescence intensities following 5-HT bath application (50 μM), fluorescence intensities were calculated from bleed-through-corrected and background subtracted green (psychLight) and red (tdTomato) fluorescence intensities using the integrated pixel intensity of a boxed region surrounding a dendrite and were represented as ΔpL2/tdT=[(pL2/tdT) peak/(pL2/tdT) baseline]. All statistics were performed across regions of interest (ROIs).

Slice Experiments

Viral Injections

Injection procedures were performed as previously described. Briefly, animals were anesthetized using 0.5%-2.5% isoflurane and mounted on a stereotaxic apparatus (Model 900). For injections into the BNST (AP: 0.3 mm, ML: 1 mm, DV: −4.35 mm from the skull), a small craniotomy (1-2 mm diameter) was performed on top of BNST injection site. The virus injection was performed using a Sub-Microliter Injection System with nanofil needles. Three hundred nL of AAV9.hSynapsin1.psych-Light2 was injected into C57/BL6J mice. Mice were allowed to recover>2 weeks to allow for sensor expression.

Brain Slices for Two-Photon Imaging

Two to 4 weeks after viral injection, mice were anesthetized with 2.5% avertin and decapitated. The heads were placed into a high-sucrose artificial cerebrospinal fluid (aCSF) solution that contained (in mM): 73 NaCl, 2.5 KCl, 2 MgCl₂, 1.25 NaH₂PO₄, 25 NaHCO₃, 24 dextrose, 0.5 CaCl₂) and 75 sucrose, saturated with 95% O₂ and 5% CO₂. The brains were removed from skull and cut (400 tm) with a vibratome (V1200s, Leica) in ice-cold high sucrose aCSF. Brain slices were incubated at 32° C. for 30 min before imaging in normal aCSF that contained (in mM): 128 NaCl, 2.5 KCl, 1 MgCl₂, 1.25 NaH₂PO₄, 25 NaHCO₃, 10 dextrose and 2 CaCl₂), saturated with 95% O₂ and 5% CO₂. Imaging was carried out at room temperature using a 2-photon microscope. The sensor was excited at 920 nm with a Ti: sapphire laser (Ultra II, Coherent) that was focused by an Olympus 40 3, 0.8NA water immersion objective. Emitted fluorescence was separated by a 525/50 nm filter set, and detected by a photomultiplier (H7422PA-40, Hamamatsu). Data were acquired and collected with ScanImage5 software. Electrical stimulation was performed with a tungsten concentric bipolar microelectrode (TM33CCINS-B, World Precision Instruments).

The area within approximately 20 tm of the electrode was imaged. Rectangular voltage pulses were applied though a 9-channel programmable pulse stimulator (Master-9, A.M.P. Instruments LTD) and a stimulus isolation unit (ISO-Flex, A.M.P. Instruments LTD). Imaging and electrical stimulation were controlled by an Axon Digidata 1550B. Field potentials were applied at 20 pulses with a duration of 0.5 s. Experiments were carried out at a scan rate of 30 (512 3 512 pixels) Hz. Image analysis was performed with ImageJ, data analyses were calculated using MATLAB and SigmaPlot 12.0. Drugs were dissolved as a stock solution in imaging HBSS buffer and diluted at 1:1000 prior to application in the perfusion system.

In vivo PsychLight Recordings

General

At the beginning of surgery, mice were anesthetized with 5% isoflurane for induction and later 1% isoflurane was used for maintenance. After induction of anesthesia, Carprofen (5 mg/kg) and Buprenorphine (1 mg/kg) were subcutaneously injected. The mouse was mounted on a stereotaxic frame. During surgery, body temperature was maintained with a heating pad. Before a sterile scalpel was used to make an incision, the hair covering the skin above the skull was removed. To have consistent horizontal alignment of the skull, bregma and lambda were leveled to be on the same z axis while two points on the surface of the skull 1.5 mm to either side of lambda were used to level the skull with regard to they axis. Following viral injection, optical fiber was implanted and secured with metabond and dental cement. Mice were monitored up to 14 days after surgery.

Viral Injection

To inject virus and implant optical fibers for fiber photometry experiments, craniotomy holes were made over the DRN, BNST, BLA, and OFC (DRN, inject with 20° angle, AP: −4.3 mm, ML: 1.1, DV: −2.85 mm; BNST, AP: 0.3 mm, ML: 1 mm, DV: −4.35 mm; BLA, AP: −1.35 mm, ML: 3 mm, DV: −4.5 mm; and OFC, AP: 2.5 mm, ML: 1.5 mm, DV: −2.5 mm). Mice were injected with 300 nL of AAV9.h-Synapsin1.psychLight2 (BNST, BLA, OFC) or AAV8.hSynapsin1.psychLight2 (DRN). Virus was injected using the Sub-Microliter Injection System with nanofil needles. The injection needle was lowered into the brain regions indicated above and infused per site at a rate of 100 nL per min. The injection volume was controlled by a microsyringe pump, which was connected to a controller. Following injection, the virus was allowed to diffuse into the tissue for an additional 10 min before the needle was withdrawn.

Optical Fiber Implantation

After viral injection, optical fibers were mounted into a stereotaxic holder and inserted into tissue targeting 50 tm above the brain regions mentioned above. A layer of Metabond was applied to the surface of the skull around the optical fiber followed by a layer of dental cement to secure the optical fiber.

Auditory Fear Conditioning

Mice were placed into a fear conditioning chamber (Med Associates) with a patch cord connected for photometric recordings. A Doric fiber photometry system was used in this study with 465 nm and 405 nm light (LED, 30 ₜW) used for generating the signal and as an isosbestic control, respectively. Each animal received 15 presentations of a 27 s tone (3000 Hz) co-terminating with a foot-shock (0.5 mA for 1.5 s) delivered at 2 min intervals. Each animal received 15 tone/foot-shock pairings over the course of 40 min, and the responses for these trials were averaged to create a single trace per animal. Data analysis was performed with custom-written script in MATLAB. In brief, 405 nm traces were fit with a bi-exponential curve, and then the fit was subtracted from the signal to correct for baseline drift. ΔF/F % was calculated as [100*(465 signal−fitted signal)/fitted signal)]. Traces were then z-scored. A heatmap was plotted using a custom MATLAB script by plotting normalized single trials of traces from all animals tested per brain region.

ROC analysis was done by a custom MATLAB script. We first calculated the baseline response from a defined a period of time (fixed measurement time point) before the shock and the sensor response from a defined period of time after the shock from the single trial data. We then calculated the probability distributions for the baseline and response periods by binning the single trial data into two histograms. We then applied a range of thresholds to the two distributions and calculated the true detection rate and false positive rate, which resulted in the ROC curve. Finally, we integrated the area under the ROC curve and approximated the d′ of the sensor as the discriminability index that had equal area under the ROC curve.

Head-Twitch Response with Fiber Photometry

Three animals were used for experiments measuring sensor activity in the prelimbic cortex. A 10 min baseline was recorded prior to compound administration (50 mg/kg 5-MeO or 4 mg/kg KETSN, i.p.) in a 5 mL/kg volume using 0.9% saline as the vehicle. To calculate the ΔF/F time series, a linear fit was applied to the 405 nm signals and aligned to the 465 nm signals. The fitted 405 nm signal was subtracted from 465 nm channels, and then divided by the fitted 405 nm signal to yield ΔF/F values. The number of head twitches were counted in 1 min intervals by 2 observers blinded to the treatment conditions and the results were averaged (interpersonnel kappas, Pearson's correlation coefficient=0.96)

Perfusion and Histology

Stock Avertin was self-made by mixing 10 g of 2,2,2-tribromoethyl alcohol and 10 mL of tert-amyl alcohol. The working stock was diluted to 1.2% (v/v) with water and shielded from light. Animals were euthanized with 125 mg/kg 1.2% Avertin (i.p.) followed by trans-cardial perfusion with ice-cold 1×phosphate buffered saline (PBS) and subsequently perfused with ice-cold 4% paraformaldehyde (PFA) in 1×PBS. After extraction of the mouse brains, samples were post-fixed in 4% PFA at 4° C. overnight. The mouse brains were cryo-protected by immersion in 10% sucrose in a 1×PBS solution overnight. Samples were next placed in 30% sucrose in a 1×PBS solution for >1 day, before embedding the samples in O.C.T. Samples were then transferred to a −80° C. freezer for long-term storage or were sliced into 50 tm sections on a cryostat (Leica Biosystems) for histology. Histology samples were imaged on Zeiss LSM 710 confocal microscope.

High Content Screening with PSYLI2 Cells

Creation of PSYLI2 Cell Line Stably Expressing PsychLight2

The psychLight2 gene was cloned into a pLVX plasmid with the EF1ₒ promotor. The plasmid was transfected into HEK293T cells together with pCMV_delta8.2 and pCMV_VSV_G in a ratio of 10:7:3 using the QIAGEN Effectene Transfection kit. After 14 h of incubation, the medium was exchanged for fresh DMEM. After an additional 48 h of incubation, the lentivirus-containing medium was collected, filtered through a 0.45 ₚm Durapore low-protein binding filter, concentrated using a Centricon-70 ultra filtration unit at 3,500 g for 50 min, and stored at −80° C. Next, confluent HEK293T cells that had been grown in 24-well plates were infected with 20 ₚL of concentrated lentivirus for 48 h. Puromycin selection was performed as described by Tandon and co-workers. Expression was assessed via fluorescence microscopy, and a single cell was selected for expansion. The new cell line, named PSYLI2, was frozen in 10% DMSO at −80° C. and then transferred to a liquid nitrogen dewar.

High-Content Imaging Experiments

Glass bottom 96-well plates (P96-1.5H-N, Cellvis) were coated with 50 pg/mL of poly-D-lysine (Sigma, P6407-5MG) and 10 pg/mL of laminin (Sigma, L2020) overnight in an incubator (37° C., 5% CO₂). Plates were washed with Dulbecco's PBS (ThermoFisher, 14190-250) and PSYLI2 cells were suspended in DMEM (Fisher, 11995073) containing 10% FBS (Fisher, 26-140-079) with 5% penicillin-streptomycin (Fisher, 15140-163) and plated at a density of 40,000 cells/well 24 h prior to each experiment. Immediately prior to an experiment, stock solutions of drugs in DMSO (10 mM) were diluted 1:100 in imaging media distributed across an empty 96-well plate (treatment plate) in triplicate following a randomized plate map. The imaging media consisted of 1×HBSS (Fisher, 14175103) containing 0.5 M MgCl₂(Sigma, M8266-1KG) and 0.5 M CaCl₂)(Sigma, C5670-50G). Cells grown in a separate 96-well plate (assay plate) were gently washed 3× with imaging media, and the wells were filled with an appropriate volume of imaging media for the respective experiment (vide infra).

Agonist Mode

For agonist mode experiments, 180 ₚL of imaging media were added to each well of the assay plate. Wells were then imaged on a Lecia DMi8 using Leica Application Suite X (V3.6.0.20104) at 40×(N.A.=0.6) with 5 regions of interest (ROI) taken per well using the default 5 ROI pattern for each well with no bias to location and no overlap of the ROIs (exposure=350 ms, LED power=80%). Next, 20 ₚL from the treatment plate was transferred to the assay plate containing a 1:1000 dilution of drug (10 ₚM as the final concentration in 0.1% DMSO). As positive, negative, and neutral controls, 5-HT (10 μM), ketanserin (10 μM), and DMSO (0.1%) were used, respectively. All final concentrations of drugs were 10 μM (0.1% DMSO) in agonist mode unless stated otherwise. After 5 min of incubation, the same sites were re-imaged using the same settings.

Once imaging was complete, the images were exported, and analyzed using self-written MATLAB script. Script will be deposit on to Github. In short, segmentation was performed on individual images and a mask highlighting the membrane of the HEK293T cells was generated. Pixel intensities were obtained from the mask-highlighted area and exported into Excel. The _{Δ}F/F values for each well were calculated using the following equation:

$$\frac{( average after drug - average before drug )}{average before drug ( baseline )}$$

These values were then used to obtain the triplicate mean (N=3).

Antagonist Mode

For antagonist mode experiments, 160 ₚL of imaging media was added to each well of the assay plate. Wells were imaged on a Lecia DMi8 using Leica Application Suite X (V3.6.0.20104) at 40×(N.A.=0.6) with 5 regions of interest (ROI) taken per well using the default 5 ROI pattern for each well with no bias to location and no overlap of the ROIs (exposure=350 ms, LED power=80%). A 100 ₚM 5-HT stock solution in DMSO was diluted 1:100 in imaging buffer. Next, 20 ₚL of this solution was added to the assay plate for a final concentration of 111 nM 5-HT (0.1% DMSO). The same 5 ROIs were imaged after 5 min of incubation. Next, 20 ₚL from the treatment plate was transferred to the assay plate for a final 1:1000 dilution of drug (10 μM drug, 100 nM 5-HT, 0.2% DMSO). All final concentrations of drugs were 10 μM with 100 nM 5-HT (0.2% DMSO) in antagonist mode unless stated otherwise. After 5 min of incubation, the same sites were re-imaged using the same settings.

Once imaging was complete, the images were exported, and analyzed using self-written MATLAB script. Script will be deposit on to Github. In short, segmentation was performed on individual images and a mask highlighting the membrane of the HEK293T cells was generated. Pixel intensities were obtained from the mask highlighted area and exported into Excel. Then the _{A}F/F values for each well were calculated using the following equation:

$$\frac{( average after drug \& 5 HT - average before 5 HT )}{average before 5 HT}$$

These values were then used to obtain the triplicate average (N=3). All imaging and incubation (both agonist and antagonist mode) were performed at ambient atmosphere and temperature.

Calculation of the Ligand Score

Compounds unlikely to bind to the sensor should produce minimal to no response in either agonist or antagonist mode. Therefore, a ligand score was calculated as:

$$Δ ( F / F ) \text{?} - [ ( Δ F / F ) \text{?} - ( Δ F / F ) \text{?} ]$$

$$\text{?} \text{indicates text missing or illegible when filed}$$

The black heatmap value indicating no effect was set to the value calculated for the vehicle control (i.e., −4.2). The maximal red and blue values were set to those calculated for a prototypical agonist (i.e., LSD, Ligand Score=21) and antagonist (i.e., MDL100907, Ligand Score=−58), respectively.

Schild Regression Analysis

A treatment plate was prepared by pre-mixing various concentrations of a non-hallucinogenic compound with increasing concentrations of 5-HT. During imaging, 180 ₘL of imaging media were added to each well of the assay plate. Wells were then imaged on a Lecia DMi8 using Leica Application Suite X (V3.6.0.20104) at 40×(N.A.=0.6) with 5 regions of interest (ROI) taken per well using the default 5 ROI pattern for each well with no bias to location and no overlap of the ROIs (exposure=350 ms, LED power=80%). Next, 20 mL from the treatment plate was transferred to the assay plate for a final 1:1000 dilution of drug. All final drug treatments contained 0.1% DMSO. After 5 min of incubation, the same sites were re-imaged using the same settings. The data analysis method was the same as in agonist and antagonist mode.

Plate Reader Screening for Compound Fluorescence

A 96-well plate (UV transparent) was prepared with 100 mL of increasing concentration of BOL-148 and bromocriptine from 10-¹² to 10-5 M together with vehicle control. The plate was read by Tecan Microplate Reader Spark® with excitation wavelength 465 nm (bandwidth 20 nm), emission wavelength 518 nm (bandwidth 20 nm), gain of 120, 5 ROI per well, total 30 flashes per well, and read at z-position 30000 mm from bottom of the plate. All settings controlled by SparkControl software, V2.3.

Antidepressant and Hallucination Related Behavior

Dendritogenesis Experiments

For the dendritogenesis experiments conducted using cultured E18 cortical neurons, timed-pregnant Sprague Dawley rats were obtained from Charles River Laboratories (Wilmington, MA). Full culturing, staining, and analysis details were performed as previously described.

Forced Swim Test (FST)

Male and female C57BL/6J mice (9-10 weeks old at time of experiment, n=6 of each sex per condition) were obtained from The Jackson Laboratory and housed 4-5 mice of the same sex/cage in a UCD vivarium following an IACUC approved protocol. After 1 week in the vivarium, each mouse was handled for approximately 1 min by a male experimenter for 3 consecutive days prior to the first FST. All experiments were conducted by the same male experimenter who performed the initial handling. During the FST, mice underwent a 6 min swim session in a clear Plexiglas cylinder (40 cm tall, 20 cm in diameter) filled with 30 cm of 24_{±}1° C. water. Fresh water was used for every mouse. After handling and habituation to the experimenter, drug-naive mice first underwent a pretest swim to more reliably induce a depressive-like phenotype in subsequent FST sessions. Immobility scores for all mice were determined after the pre-test and mice were assigned to treatment groups to generate groups with similar mean immobility scores used in the following two FST sessions. The next day, the animals received injections (i.p.) of AAZ-A-154 (20 mg/kg), ketamine (3 mg/kg) as the positive control, or vehicle (saline). After 30 min, the animals were subjected to the FST, dried with a towel, and then returned to their home cages. One week later, the FST was performed to assess the sustained effects of the drugs. All FSTs were performed between the hours of 0800 and 1300 h. The experiments were divided into two cohorts either of all males or females and conducted on different days. Experiments were video-recorded and manually scored offline by an experimenter blinded to treatment conditions. Immobility time-defined as passive floating or remaining motionless with no activity other than that needed to keep the mouse's head above water—was scored for the last 4 min of the 6 min trial.

Head-Twitch Response (HTR) and Locomotion Assays

The HTR assay was performed as described previously using both male and female C57BL/6J mice (2 male and 2 female=4 total per treatment). The mice were obtained from The Jackson Laboratory (Sacramento, C.A.) and were approximately 8-weeks old at the time of the experiments. Compounds were administered (5 mL/kg, i.p.) using 0.9% saline as the vehicle. After injection, animals were placed into an empty cage (8″×13″×5″) and HTRs were videotaped, scored later by two blinded observers, and the results were averaged (interpersonnel kappas, Pearson correlation coefficient>0.91). Locomotion was assessed using AnyMaze automated tracking software.

Sucrose Preference

Adult male and female wild-type (WT) and VMAT2 heterozygous (VMAT2-HET) mice were used for these experiments, and they were housed in a humidity- and temperature-controlled room on a 14:10 h light:dark cycle. Mice were housed individually 48 h prior to the experiment with ad libitum access to chow and water. For each day's experiment, bottles were prepared with water or a 1% sucrose solution and these were weighed just prior to the test. Two h prior to the beginning of the dark cycle, the home-cage water bottle was removed. One h after onset of the dark cycle, a pair of bottles was placed into the home-cage. The mouse was given 2 h to drink, after which the bottles were removed and weighed immediately. Approximately 1 h later, the home-cage water bottle was returned. This procedure was repeated daily with the water-water (W-W) pairing until the mouse showed stable drinking volumes over 3 consecutive days without any side-bias. Once criterion was achieved, the mouse was presented with the water-sucrose (W-S) pairing. The next day (day 1), mice were administered an acute injection of AAZ-A-154 (15 mg/kg, i.p.) and 5 min later were given the W-S pairing (i.e., day 1). Subsequent W-S pairings were presented on days 2 and 4, and then at 4-day intervals. Preference for the sucrose bottle was calculated as the volume of sucrose consumed minus the volume of water consumed, divided by the total volume of liquid consumed. Preference scores approaching “0” indicated no preference for sucrose or water, whereas positive scores signified a preference for sucrose and negative scores denoted a preference for water.

Compound Synthesis

Synthesis of Previously Uncharacterized Compounds

The syntheses and characterization of most compounds used in this study have been reported previously. Here, we provide synthetic procedures and characterization data (Data S2) for AAZ-A-154 and LED-C-233, as they have not been previously described.

### (R)-2-(dimethylamino)propan-1-ol

To an ice-cold solution of R-alaninol (4.93 g, 65.6 mmol) and glacial acetic acid (18.9 mL, 328 mmol, 5.0 equiv) in MeOH (328 mL) was added sodium cyanoborohydride (9.075 g, 144 mmol, 2.2 equiv) followed by 37% formaldehyde, (13.8 mL, 171 mmol, 2.6 equiv). The reaction was stirred at room temperature for 12 h before being concentrated under reduced pressure. The residue was diluted with glycerol (100 mL) and distilled under reduced pressure to yield the pure compound as a colorless oil (6.5 g, 96%), which was used without further purification.

### (R)-1-chloro-N,N-dimethylpropan-2-amine hydrochloride

To an ice-cold solution of SOCl₂(2.1 mL, 29 mmol, 1.1 equiv) was added (R)-2-(dimethylamino)propan-1-ol (2.7 g, 26 mmol). The mixture was heated to reflux for 4 h before being concentrated under reduced pressure to yield the desired product as a white solid (3.92 g, 95%), which was used without further purification.

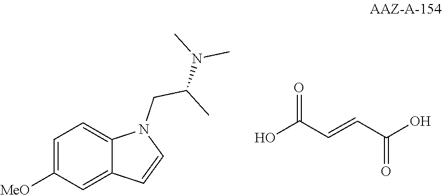 [SMILES: COc1ccc2c(ccn2C[C@@H](C)N(C)C)c1 ; O=C(O)/C=C/C(=O)O]

To a solution of 5-methoxyindole (441 mg, 3.00 mmol) in DMSO (7.5 mL) was added (R)-1-chloro-N,N-dimethylpropan-2-amine hydrochloride (664 mg, 4.20 mmol, 1.4 equiv), potassium iodide (697 mg, 4.2 mmol, 1.4 equiv), and potassium tert-butoxide (0943 mg, 8.40 mmol, 2.8 equiv). The reaction mixture was stirred for 24 h, before being diluted with 1.0 M NaOH_{(aq)} (750 mL). The aqueous phase was extracted with DCM (3 3 100 mL). The organic extracts were combined, dried over Na₂SO₄, filtered, and concentrated under reduced pressure to yield a colorless oil, which was purified by flash chromatography (9:1 DCM/MeOH with 1% ammonium hydroxide). The purified oil was dissolved in CHCl₃(3 mL) and added dropwise to a boiling solution of fumaric acid (253 mg, 2.18 mmol, 1.0 equiv) in THF (10 mL). The mixture was concentrated under reduced pressure to yield the desired product as the 1:1 fumarate salt (758 mg, 73%). ¹H NMR (600 MHz, DMSO-d₆) 7.37 (d, 1H, J=8.8 Hz), 7.30 (s, 1H), 7.03 (s, 1H, J=3.1 Hz), 6.76 (d, 1H, J=8.8 Hz), 6.61 (s, 2H), 6.32 (s, 1H), 4.25 (dd, 1H J=6.3, 7.8 Hz), 4.02 (dd, 1H, J=6.3, 7.8 Hz), 3.74 (s, 3H) 3.11 (q, 1H, J=6.3, 6.6, Hz), 2.30 (s, 6H), 0.84 (d, 3H, J=6.6 Hz). ¹³C NMR (100 MHz, CD₃OD) 171.0, 155.8, 136.1, 132.8, 130.9, 129.7, 113.3, 111.2, 103.8, 103.3, 61.6, 56.2, 47.5, 39.9, 11.7 ppm.

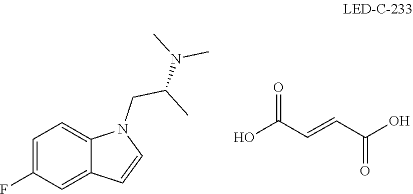 [SMILES: C[C@H](Cn1ccc2cc(F)ccc21)N(C)C ; O=C(O)/C=C/C(=O)O]

To a solution of 5-fluoroindole (100 mg, 0.739 mmol) in DMSO (1.90 mL) was added (R)-1-chloro-N,N-dimethylpropan-2-amine hydrochloride (128 mg, 0.814 mmol, 1.1 equiv), potassium iodide (135 mg, 0.814 mmol, 1.1 equiv), and potassium hydroxide (166 mg, 15.8 mmol, 5.0 equiv). The reaction mixture was stirred for 24 h, before being diluted with 1.0 M NaOH_{(aq)} (100 mL). The aqueous phase was extracted with DCM (3 3 25 mL). The organic extracts were combined, dried over Na₂SO₄, filtered, and concentrated under reduced pressure to yield a colorless oil, which was purified by flash chromatography (9:1 DCM/MeOH with 1% ammonium hydroxide). The purified oil was dissolved in acetone (2 mL) and added dropwise to a boiling solution of fumaric acid (48.1 mg, 0.409 mmol, 1.0 equiv) in acetone (5 mL). The mixture was concentrated under reduced pressure to yield the desired product as the 1:1 fumarate salt (111 mg, 54%). ¹H NMR (600 MHz, CD₃OD) 7.49 (m, 1H), 7.34 (d, 1H, J=3.2 Hz), 7.25 (dd, 1H, J=2.5, 9.3 Hz), 6.98 (td, 1H, J=2.5. 9.3 Hz), 6.72 (s, 2H), 6.53 (d, 1H, J=3.2 Hz), 4.63 (dd, 1H J=5.7, 8.9 Hz), 4.35 (dd, 1H, J=5.7, 8.9 Hz), 3.86 (m, 1H) 2.84 (s, 6H), 1.21 (d, 3H, J=6.7 Hz). ¹³C NMR (100 MHz, CD₃OD) _{d} 171.0, 160.2, 158.7, 136.1, 134.2, 131.1, 130.8, 130.7, 111.5, 111.4, 111.3, 111.1, 106.7, 106.5, 103.6, 103.5, 61.5, 47.6, 40.0, 11.6 ppm.

Quantification and Statistical Analysis

Treatments were randomized, and the data were analyzed by experimenters blinded to the treatment conditions. Statistical analyses were performed using GraphPad Prism (version 8.1.2) unless noted otherwise. All comparisons were planned prior to performing each experiment. The sucrose preference and the volume of liquid consumed in the anhedonia test were analyzed separately by repeated-measures ANOVA using a within subjects' effects of days and a between subjects' effects of genotype with SPSS 27 programs (IBM SPSS Statistics, Chicago, IL). Post hoc analyses were by Bonferroni corrected pairwise comparisons. A p<0.05 was considered significant. Data are represented as mean±SEM, unless otherwise noted, with asterisks indicating *p<0.05, **p<0.01, ***p<0.001, and ****p<0.0001. Details of the statistical tests are displayed in Table S2.

Development of psychLight

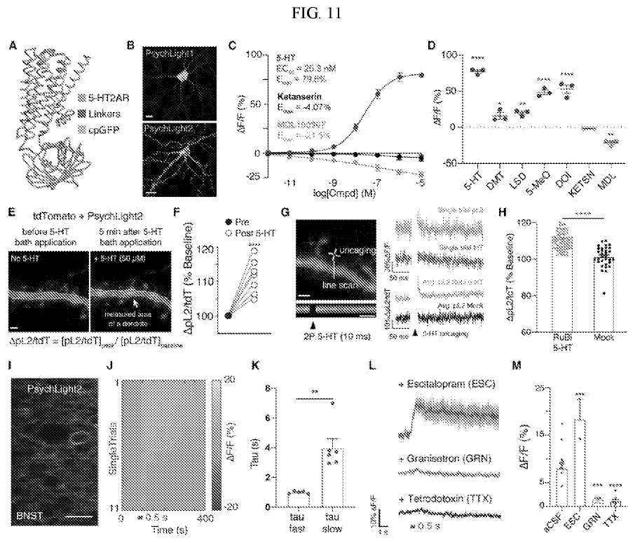

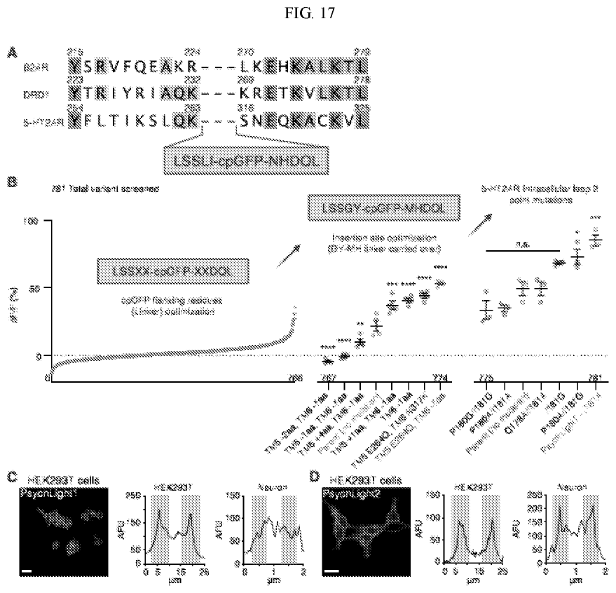

To develop a sensor capable of reporting ligand-induced conformations of the human 5-HT2AR, we replaced the third intracellular loop (IL3) of the 5-HT2AR with a circularly permuted green fluorescent protein (cpGFP) inserted between Lys263 and Ser316 (FIG. 17A). The dynamic range in response to the endogenous ligand 5-HT was maximized by screening linker compositions between cpGFP and 5-HT2AR, optimizing the insertion site of cpGFP, and introducing key point mutations (FIG. 17B) (Patriarchi et al., 2018). We named the top-performing variant psychLight1 (FIG. 11A). To further improve membrane localization in neurons, we fused an endoplasmic reticulum (ER) export motif (FCYENEV) (Stockklausner et al., 2001) to the C terminus of psychLight1, yielding a version (i.e., psychLight2) with improved membrane expression in both HEK293T cells and neurons (FIG. 11B, FIGS. 17C and D).

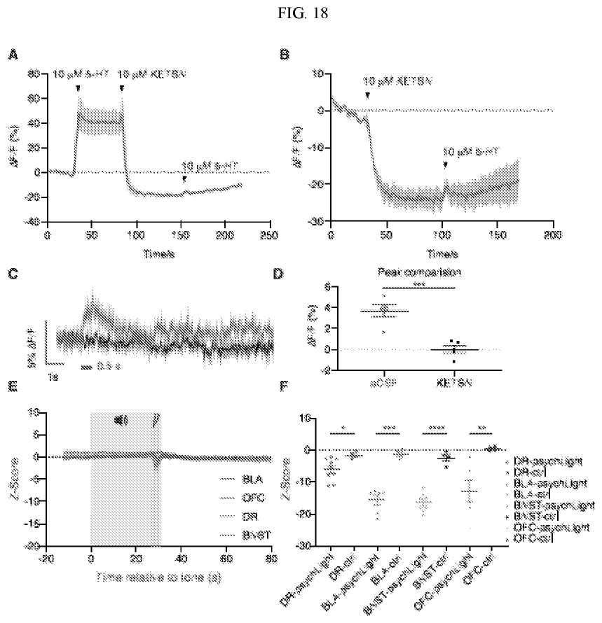

We next investigated the pharmacological responses of the sensor. When psychLight1 is expressed in HEK293T cells, 5-HT activates the sensor with a half maximal effective concentration comparable to values obtained using assays designed to measure G protein and _{b}-arrestin activation (Wacker et al., 2017) (FIG. 11C). Moreover, other agonists were able to effectively increase the sensor's fluorescence intensity to varying degrees (FIG. 11D). In contrast, the traditional 5-HT2AR antagonists ketanserin and MDL100907 either had minimal effect on psychLight1 fluorescence or slightly quenched the sensor (FIGS. 11C and D), and ketanserin was able to block 5-HT-induced activation of psychLight in HEK293T cells (FIGS. 18A and B). Taken together, these findings demonstrated that psychLight can convert ligand-induced conformational changes of the 5-HT2AR into fluorescence readouts, suggesting that psychLight may be uniquely suited for detecting specific conformations of the receptor induced by ligands.

Two-Photon Imaging of Endogenous Serotonin Dynamics Ex Vivo and In Vivo

To assess the utility of psychLight2 for measuring endogenous serotonin transients, we characterized the sensitivity and kinetics of the sensor using two-photon imaging in cultured and acute brain slices. Approximately 3 weeks after infection with AAV9.hSynapsin1.psychLight2 into organotypic cortical slice cultures and biolistic transfection of a red cell-fill fluorescent protein, tdTomato, we imaged layer 2/3 pyramidal neurons using two-photon time-lapse imaging and line-scan acquisition mode (3.3 lines/ms). Bath application of 5-HT (50 ₘM) led to a significant increase in the ratio of green (psychLight2 signal; pL2) to red (tdTomato signal; tdT) fluorescence intensities (FIGS. 11E and F). Focal uncaging of RuBi-5-HT at apical dendrites (single 10-ms pulse at 810 nm) evoked a rapid increase in psy-chLight fluorescence that returned to baseline within millisec-onds (Tau_{off}=5.4±0.9 ms) (FIGS. 11G and H). In contrast, no increase in fluorescence was observed in response to a mock stimulus.

Next, we examined the ability of psychLight2 to report time-dependent changes in 5-HT dynamics using an acute slice preparation. Three weeks after injection of AAV9.hSynapsin1.psych-Light2 into the bed nucleus of the stria terminalis (BNST) (FIG. 11I), we performed two-photon imaging in frame-scan mode (33 Hz) and triggered endogenous 5-HT release by electrical stimulation. The sensitivity of psychLight2 was sufficient to detect electrically evoked 5-HT release in single trials (d^{p}=234.2) (FIG. 11J). Interestingly, we noticed two types of responses that differed in their amplitudes and decay rates (ΔF/F=4.7%+1.5%, Tau_{off} fast=0.997_{±}0.038 s and ΔF/F=9.7%_{±}1.2%, Tau_{off} slow=3.998_{±}0.610 s) (FIGS. 11J and K). The amplitude of the psychLight2 response could be enhanced by incubation with 50 ₘM escitalopram, a blocker of the 5-HT transporter (SERT) (ΔF/F=18.4%_{±}4.3%) (FIG. 11L). Importantly, application of either the 5-HT₃ receptor antagonist granisetron (10 ₘM) (Ko et al., 2016), the sodium channel blocker tetrodotoxin (1 mM), or ketan-serin (10 mM) was sufficient to block psychLight fluorescence in response to electrically-evoked 5-HT transients (FIG. 11L, M, FIGS. 18C and D).

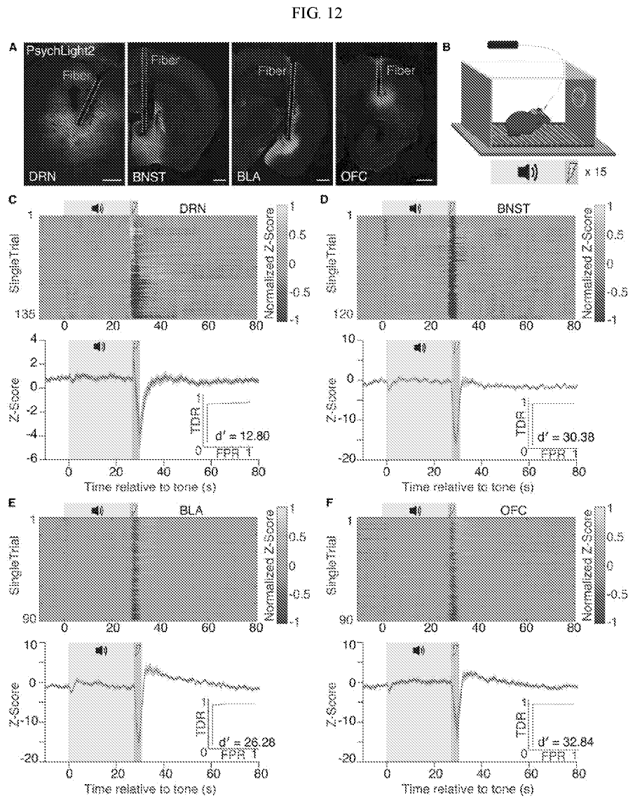

To determine if psychLight2 could measure 5-HT dynamics in vivo, we employed a fear conditioning paradigm coupled with fiber photometry in freely behaving mice. First, AAV.hSy-napsin1.psychLight2 was injected into the BNST, the basolateral amygdala (BLA), the dorsal raphe nucleus (DRN), or the orbito-frontal cortex (OFC) along with implantation of an optical fiber (FIG. 12A). After 2-3 weeks to allow full expression of the sensor, we measured 5-HT transients during an auditory fear conditioning experiment consisting of 15 presentations of a 30 s tone co-terminating with a 1.5-s foot-shock (0.5 mA) (FIG. 12B). In the DRN, we observed a robust increase in fluorescence intensity immediately after the onset of foot-shock (FIG. 12C), followed by a sharp decline during the shock. These results are consistent with Ca²⁺ transients recorded in the DRN using GCaMP6 during auditory fear conditioning (Ren et al., 2018). In the BNST, we observed an immediate decrease in fluorescence following foot-shock that returned to baseline within 4 s (FIG. 12D). A similar initial reduction in fluorescence was observed in both the BLA and OFC; however, in these brain regions, the initial decrease in sensor activity was followed by a considerable rise in the fluorescence signal following the shock (FIGS. 12E and F). Serotonin dynamics were reliably detected across individual trials of the fear conditioning experiments (d⁰=12.80, 30.38, 26.28, and 32.84 for the DRN, BNST, BLA, and OFC, respectively). To further demonstrate that changes in psy-chLight fluorescence during fear conditioning are specific to endogenous 5-HT2AR ligands, we injected AAV.hSynapsin1.-psychLight0 into all four brain regions. PsychLight0 has a key point mutation (D155A) that completely prevents agonist binding. Unlike experiments using psychLight2, we did not observe significant changes in psychLight0 fluorescence following foot-shock (FIGS. 18E and F), indicating that psychLight2 detects endogenous agonists in freely behaving animals.

PsychLight Activity Differentiates Hallucinogenic and Non-Hallucinogenic Drugs

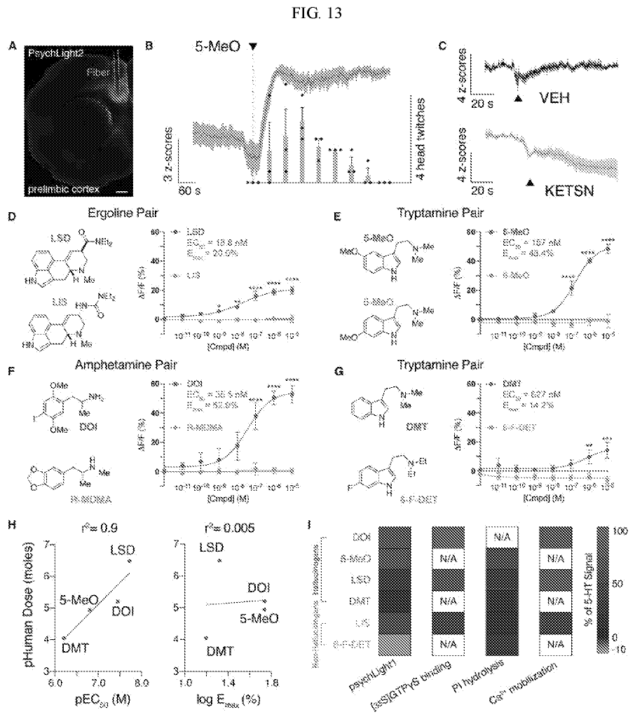

We next sought to determine if the sensor could faithfully report 5-HT2AR activation in vivo following systemic administration of an exogenous agonist. We chose to use 5-methoxy-N,N-dimeth-yltryptamine (5-MeO-DMT or 5-MeO), because it produces a robust head-twitch response (HTR) (Dunlap et al., 2020)—a mouse behavior induced by hallucinogenic 5-HT2AR ligands (Halberstadt et al., 2020; Hanks and Gonzalez-Maeso, 2013). Three weeks after injection of AAV9.hSynapsin1.psychLight2 into the prelimbic cortex, we administered 5-MeO-DMT (50 mg/kg, intraperitoneal [i.p.]) and measured psychLight2 response using fiber photometry (FIGS. 13A and B). Within 1 min of drug administration, we observed a sharp increase in fluorescence along with a concomitant increase in HTR. After several minutes, the psychLight signal stabilized and remained elevated while head twitch frequency decreased (FIG. 13B). In contrast, when mice were administered vehicle or the 5-HT2AR antagonist ketanserin (KETSN, 4 mg/kg, i.p.), psy-chLight fluorescence remained unchanged or decreased, respectively (FIG. 13C). These data suggest that psychLight is sensitive to both agonist- and antagonist-induced conformational changes in vivo.

We next assessed the sensor's ability to differentiate between known hallucinogenic agonists and structurally similar non-hallucinogenic analogs. We tested several pairs of hallucinogenic and non-hallucinogenic congeners representing the ergoline, trypt-amine, and amphetamine classes of psychedelics. We chose these compounds because the propensity of these drugs to produce hallucinations in humans was known (Benes et al., 2006; Dunlap et al., 2018; Halberstadt et al., 2020; Kalir and Szara, 1963) or inferred from data using well-established rodent models of 5-HT2AR-induced hallucinations (Hanks and Gonzalez-Maeso, 2013), such as rat drug discrimination (DD) (Glennon et al., 1983) and mouse HTR assays (Dunlap et al., 2020), which correlate exceptionally well with hallucinogenic potency in humans (Halberstadt et al., 2020).

All four hallucinogenic compounds activated psychLight1 when expressed in HEK293T cells, with half maximal effective concentrations ranging from 18.8-627 nM (LSD, EC₅₀=18.8 nM, Eₘₐₓ=20.0%; 5-MeO, EC₅₀=157 nM, Eₘₐₓ=48.4%; DOI, EC₅₀=35.5 nM, Eₘₐₓ=52.9%; and DMT, EC₅₀=627 nM, Eₘₐₓ=12.4%). In sharp contrast, none of the non-hallucinogenic congeners were able to increase the sensor's response, even at concentrations as high as 10 mM (FIG. 13D-G). By running the assay in antagonist mode, we were able to demonstrate that non-hallucinogenic compounds such as lisuride (LIS) and 6-MeO-DMT (6-MeO) are capable of binding to the receptor despite lacking efficacy (FIGS. 19A and B). The large Eₘₐₓ differences between the hallucinogenic and non-hallucinogenic compounds within a given pair are remarkable given the extremely high degree of structural similarity between the paired molecules.

PsychLight1 potencies, but not efficacies, correlate exceptionally well with hallucinogenic potencies in humans (r²=0.9) (FIG. 13H). This strong correlation is noteworthy considering the error associated with estimating hallucinogenic potencies in humans and the fact that our cellular assay does not account for potential differences in pharmacokinetics. Furthermore, ligand activation of psychLight1 appears to be distinct from other measures of 5-HT2AR activation including phosphoinositide hydrolysis (Cussac et al., 2008), Gq activation (Rabin et al., 2002), and calcium mobilization (Cussac et al., 2008) (FIG. 13I).

Development of a psychLight-Based Medium-Throughput Pharmacological Assay

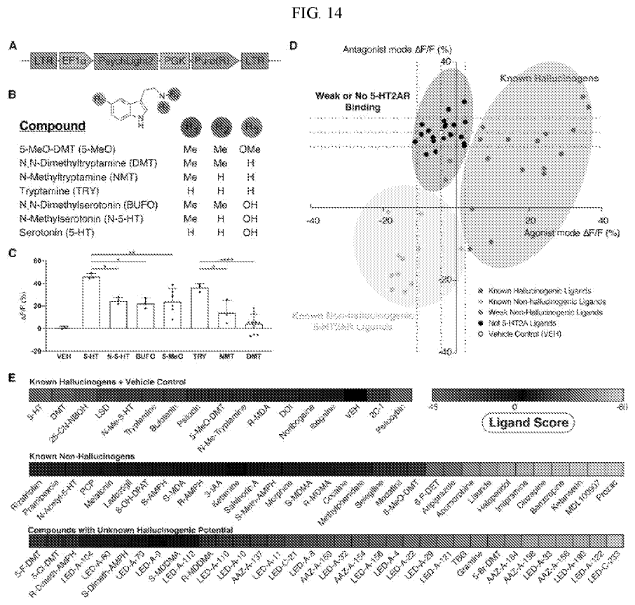

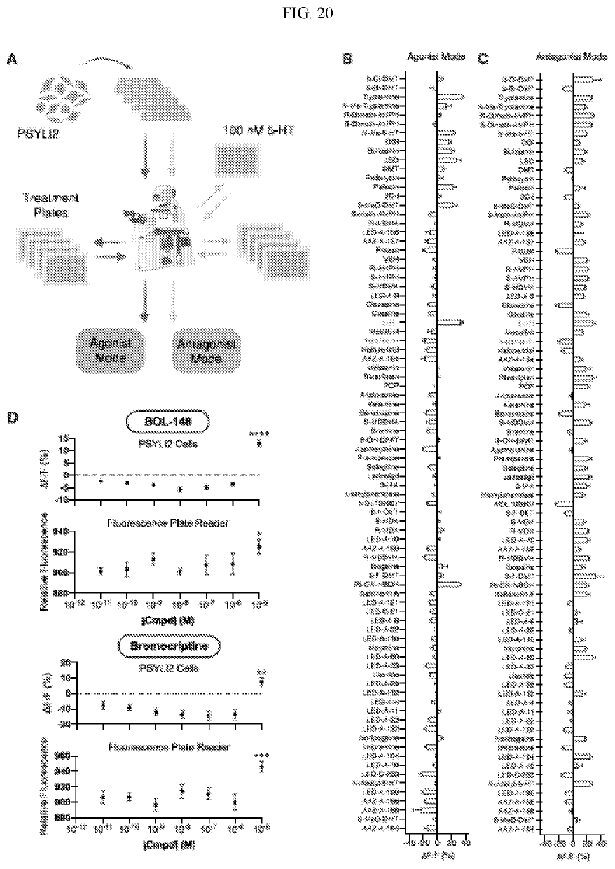

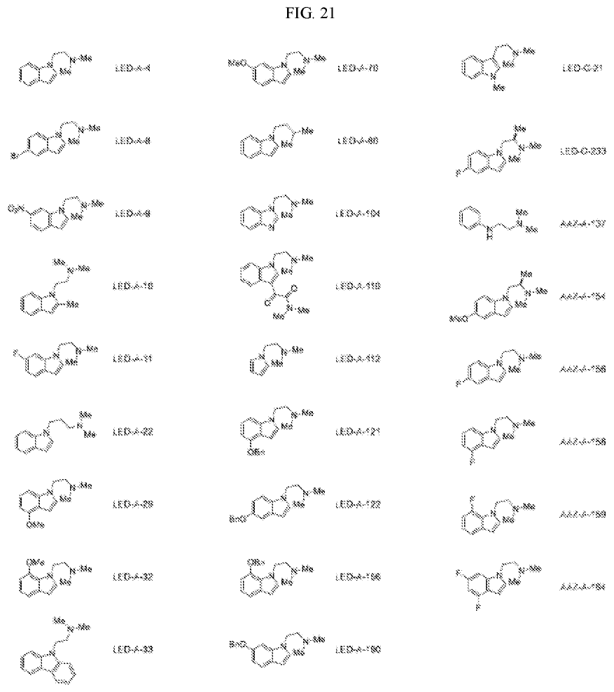

To enable medium-throughput identification of hallucinogenic designer drugs of abuse as well as non-hallucinogenic therapeutics targeting 5-HT2ARs, we developed a screening platform based on wide-field high content imaging of a HEK293T cell line stably expressing psychLight2 (PSYLI2) under the EF1 promoter (FIG. 14A and FIG. 20A). Relative responses were similar using either a confocal microscope or high content imager (FIG. 19C). A Z-factor (Zhang et al., 1999) was generated using serotonin and ketanserin as positive and negative controls, respectively (Z factor=0.6, n=42) (FIG. 19D).

To assess the sensitivity of this assay, we first tested a panel of ligands with similar molecular structures to 5-HT (FIG. 14B). We observed that subtle differences in ligand structure can significantly modulate the fluorescence signal generated by PSYLI2 cells (FIG. 14C). Most notably, increasing N-methylation tends to reduce the magnitude of the sensor response (e.g., 5-HT: ΔF/F=46.3%_{±}1.4%; N-methylserotonin, N-5-HT: ΔF/F=24.6%_{±}1.7%; and N,N-dimethylserotonin, BUFO: ΔF/F=22.3%_{±}2.8%), which is consistent with a structure-activity relationship previously reported for 5-HT2AR-induced accumulation of [³H]inositol phosphates (Ebersole et al., 2003). Surprisingly, the hydroxyl substituent of 5-HT does not appear to be necessary for achieving full agonism as tryptamine produces a robust response (FIG. 14C).

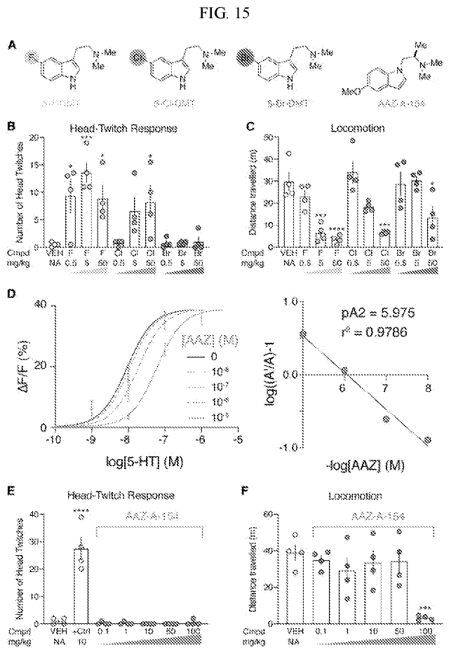

Next, we screened a library of eighty-three compounds consisting of known hallucinogens (as defined by human data or predicted based on the mouse HTR and/or DD assays), known non-hallucinogenic 5-HT2AR ligands, psychoactive drugs with unknown 5-HT2AR affinity, and compounds from our medicinal chemistry program (FIG. 20B, C, and FIG. 15). When the assay was performed in agonist mode (FIG. 14D, abscissa), serotonergic hallucinogens reliably gave a response of greater than +1 SD from the vehicle control (FIG. 14D and FIG. 20B). Non-hallucinogenic 5-HT2AR ligands did not activate the sensor in agonist mode but decreased fluorescence in antagonist mode (FIG. 14D, ordinate). Compounds that do not bind to the 5-HT2AR did not produce a response in either agonist or antagonist mode.

When screened at 10 μM using PSYLI2 cells, 2-bromolysergic acid diethylamide (BOL-148) and bromocriptine produced unexpected fluorescence signals, because these compounds are widely believed to be non-hallucinogenic. Given that both compounds contain a two-bromoindole structural motif, we suspected that the inherent fluorescence of these molecules was resulting in false-positive signals. Therefore, we performed concentration-response experiments in PSYLI2 cells using a high content imager and under cell-free conditions using a fluorescence plate reader (FIG. 20D). The results confirmed our hypothesis that the signal from BOL-148 and bromocriptine at 10 μM was due to the inherent fluorescence of these compounds, and not due to activation of the sensor.

By running the assay in both agonist and antagonist mode (i.e., 100 nM 5-HT with 10 μM test compound), we were able to distinguish between non-hallucinogenic ligands of the 5-HT2AR and compounds that do not bind to the receptor (FIG. 14D, E, and FIG. 20C). We combined data from agonist and antagonist modes to define a “ligand score”; positive and negative ligand scores indicated likely hallucinogenic and non-hallucinogenic ligands of the 5-HT2AR, respectively, whereas values close to zero indicated compounds that were unlikely to be 5-HT2AR ligands (FIG. 14E). For example, the ligand scores for LSD and lisuride were 23.0 and −42.3, respectively. In contrast, non-serotonergic hallucino-gens/dissociatives such as salvinorin A, ketamine, and phencyclidine displayed ligand scores close to 0 (FIG. 14E).

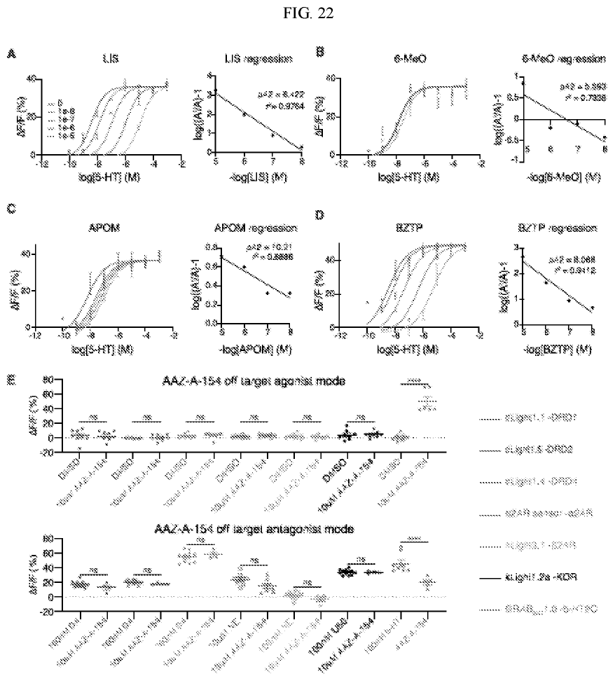

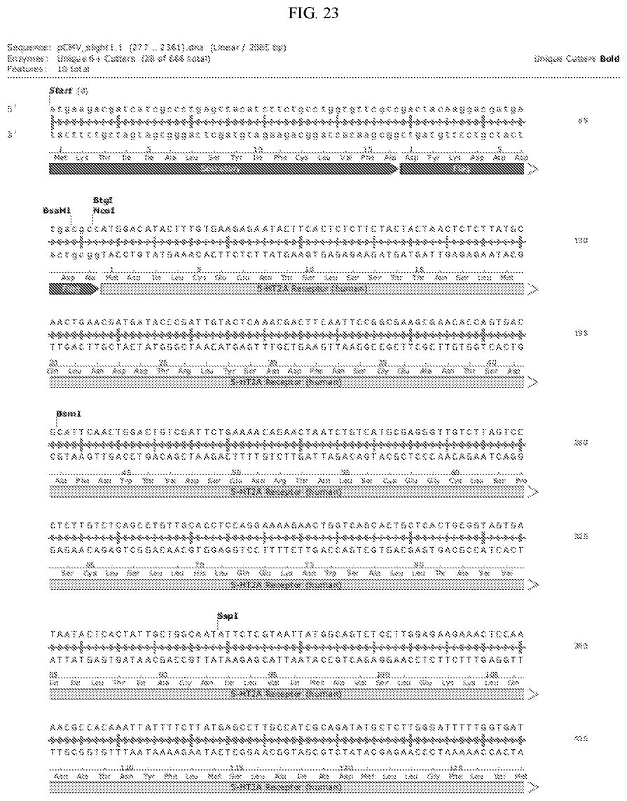

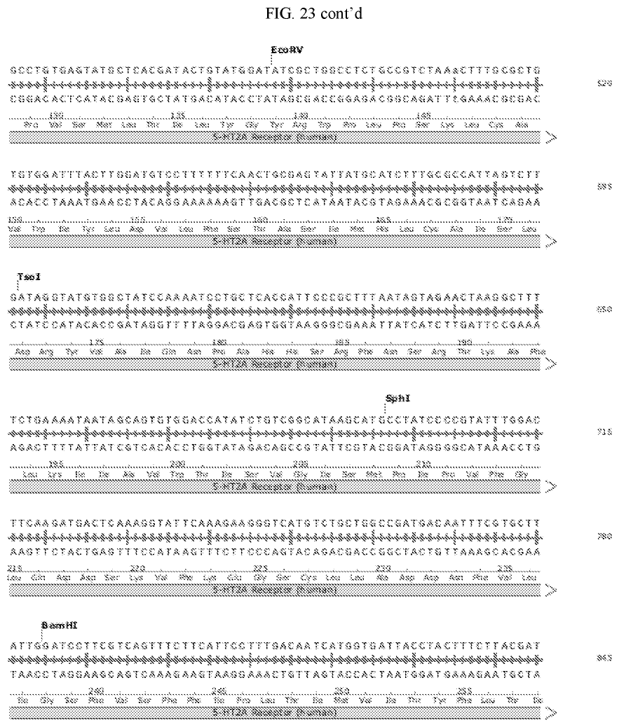

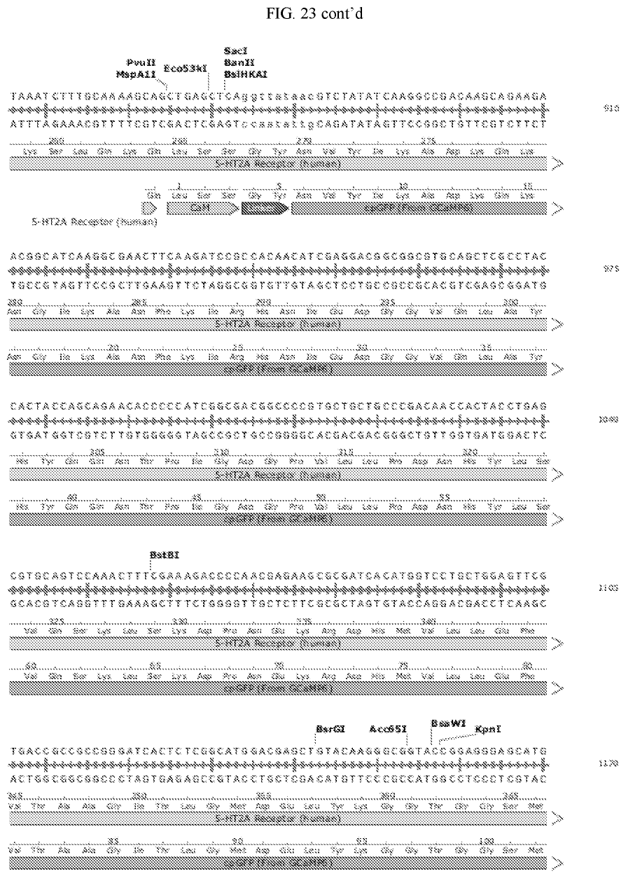

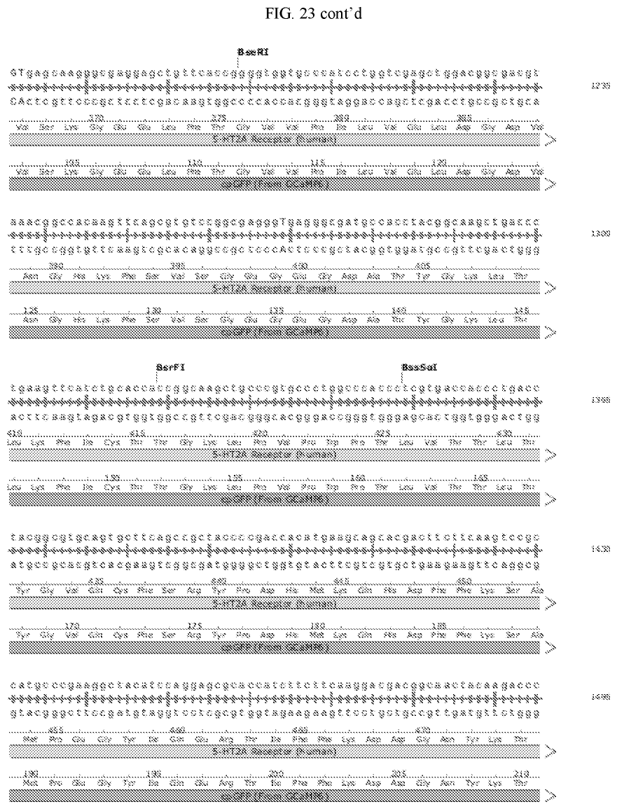

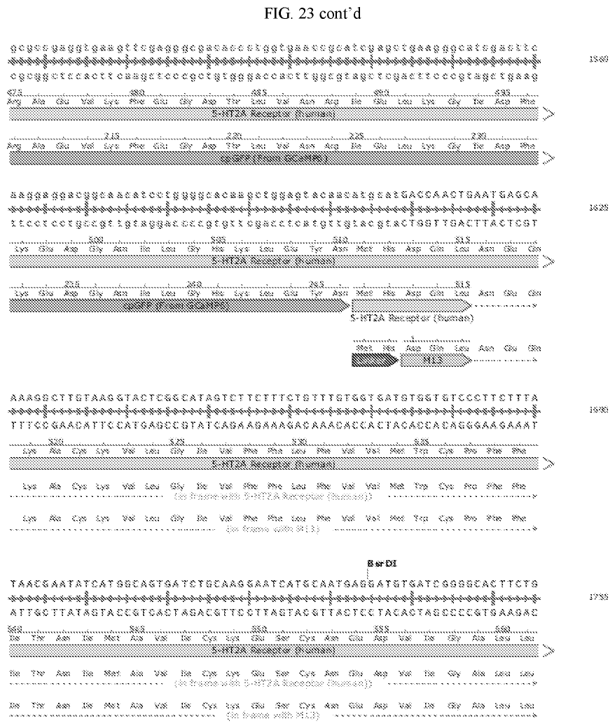

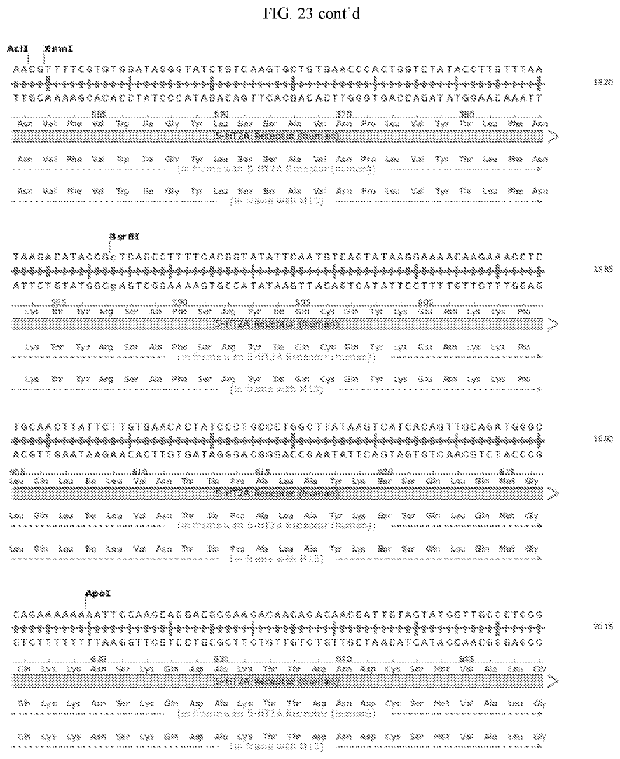

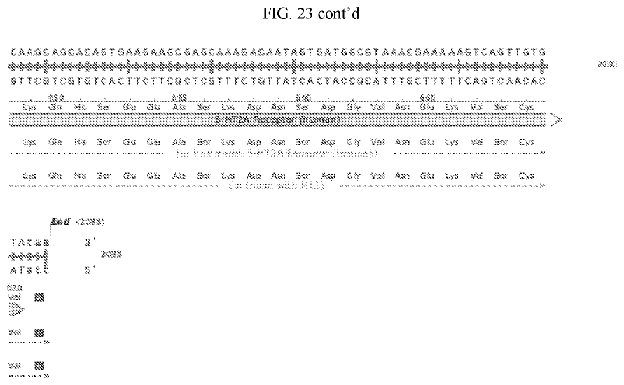

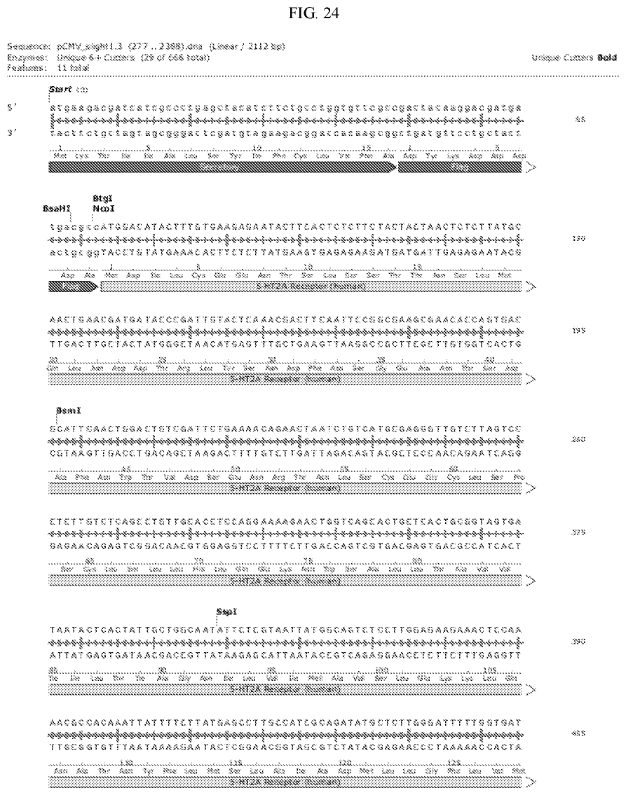

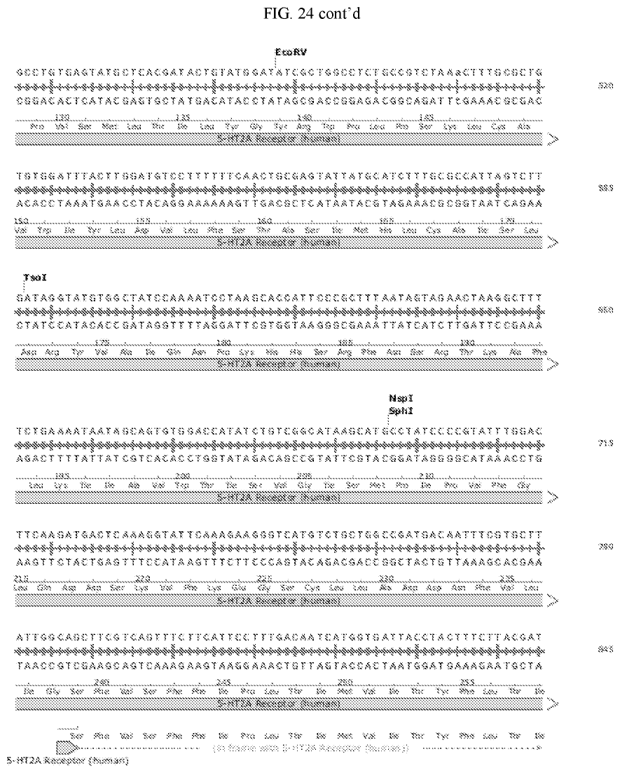

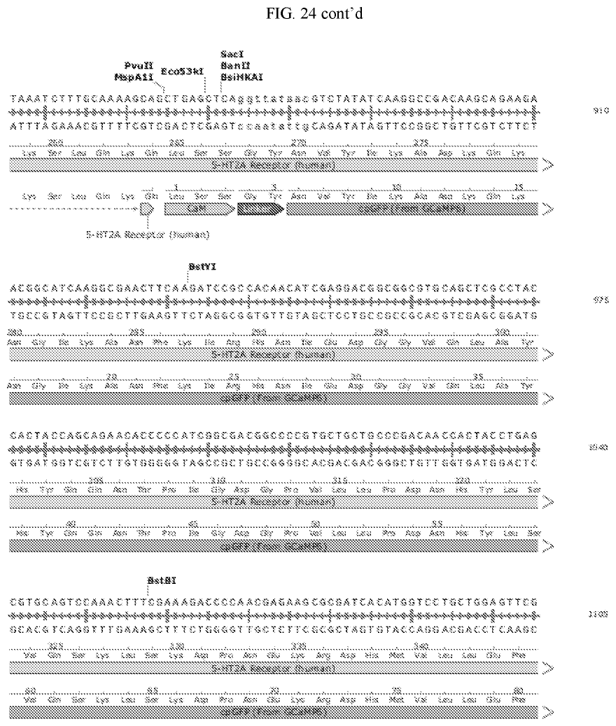

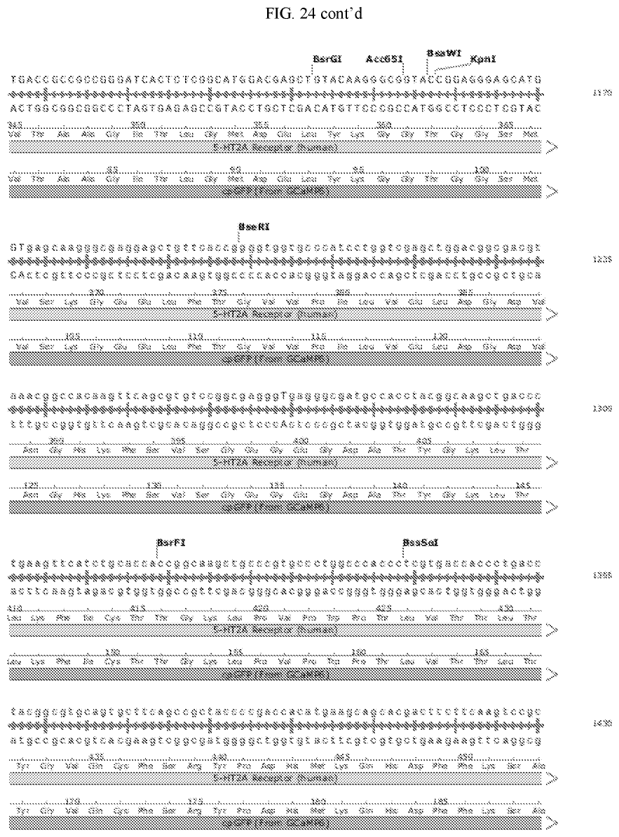

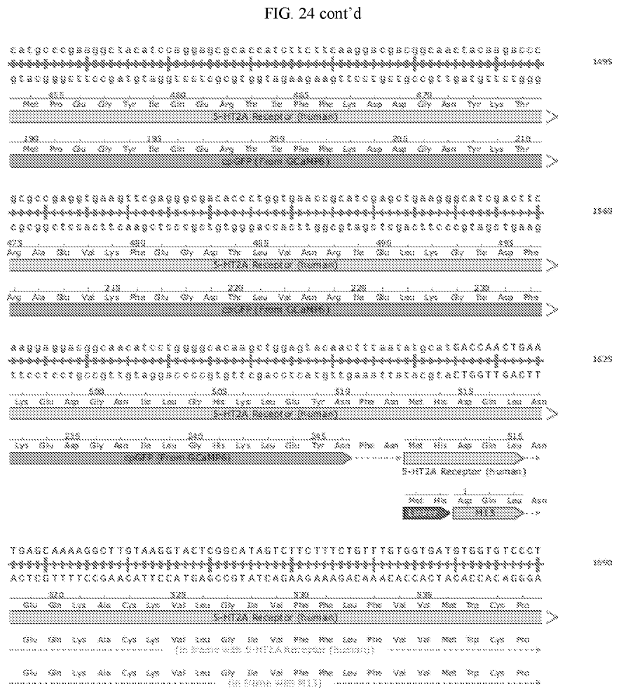

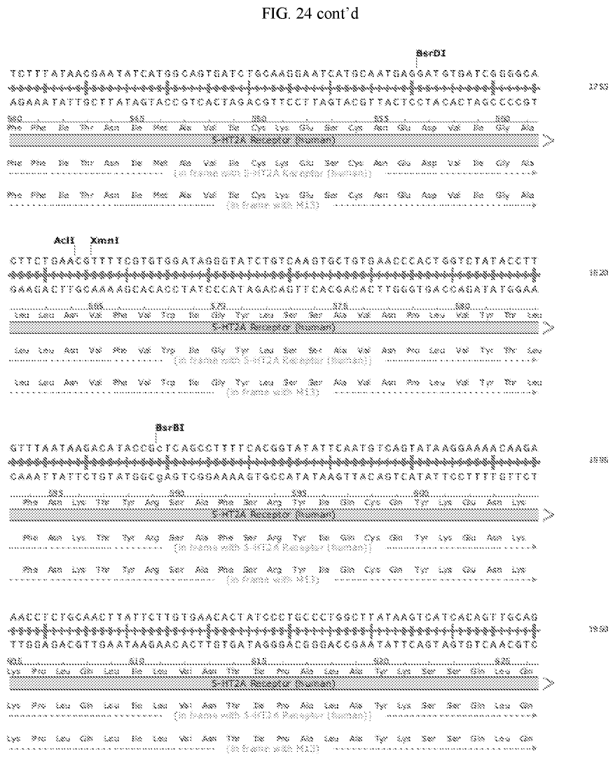

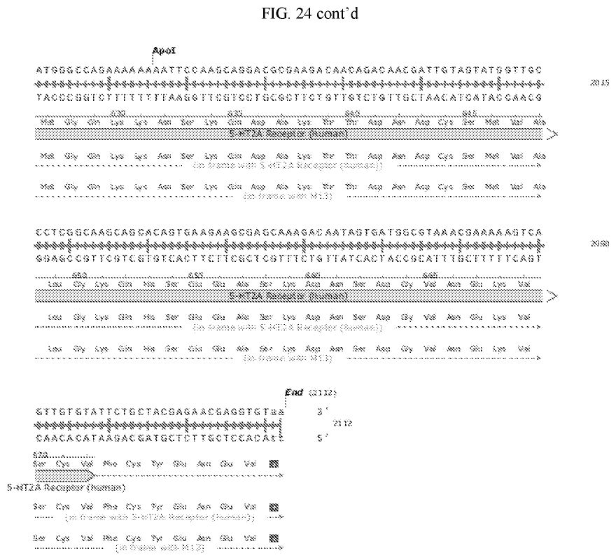

Finally, to further characterize the pharmacological profiles of non-hallucinogenic ligands, we performed Schild regression analysis for several compounds with negative ligand scores (FIG. 22A-D). The pA2 values indicate that LIS, apomorphine, and benztropine are potent psychLight competitive antagonists, whereas 6-MeO is significantly less potent (FIG. 22A-D).

PsychLight Accurately Predicts the Hallucinogenicpotentials of Designer Drugs

We next screened a small library consisting of thirty-four compounds with unknown hallucinogenic potentials (FIG. 14E). By assessing ligand scores, we predicted that the smaller 5-F-DMT and 5-Cl-DMT would be hallucinogenic, while the larger 5-Br-DMT would not (FIG. 14E and FIG. 15A). To confirm this prediction in vivo, we performed a three-point dose-response study measuring HTR (FIG. 15B). As expected, both 5-F-DMT and 5-Cl-DMT produced robust HTRs, while 5-Br-DMT failed to induce HTRs at any dose (FIG. 15B). Interestingly, the effects of the compounds on locomotion and the HTR were not correlated (FIG. 15C). The 5-halo-DMT series really highlights the power of psychLight for detecting profound functional differences between compounds that share a high degree of structural similarity.

Next, we sought to use psychLight to identify non-hallucinogenic 5-HT2AR ligands occupying previously unknown chemical space. Because AAZ-A-154 (FIG. 15A) had never been reported in the literature and exhibited a favorable ligand score, we subjected it to further testing. Schild regression analysis revealed that AAZ-A-154 functions as a psychLight competitive antagonist (FIG. 15D). Using a panel of GPCR-based sensors (e.g., dopamine, adrenergic, opioid, and serotonin receptors) (Patriarchi et al., 2018; Wan et al., 2021) in both agonist and antagonist mode, we observed that AAZ-A-154 exhibits high selectivity for 5-HT2 receptors (FIG. 22E). To assess the hallucinogenic potential of AAZ-A-154 in vivo, we performed HTR experiments across multiple doses in mice. As expected, AAZ-A-154 failed to produce any head-twitches, even up to doses as high as 100 mg/kg (FIG. 15E). However, a high dose of AAZ-A-154 decreased locomotion (FIG. 15F), indicating that this compound can still impact behavior without producing hallucinogenic effects.

Characterizing the Antidepressant-Like Effects of AAZ-A-154

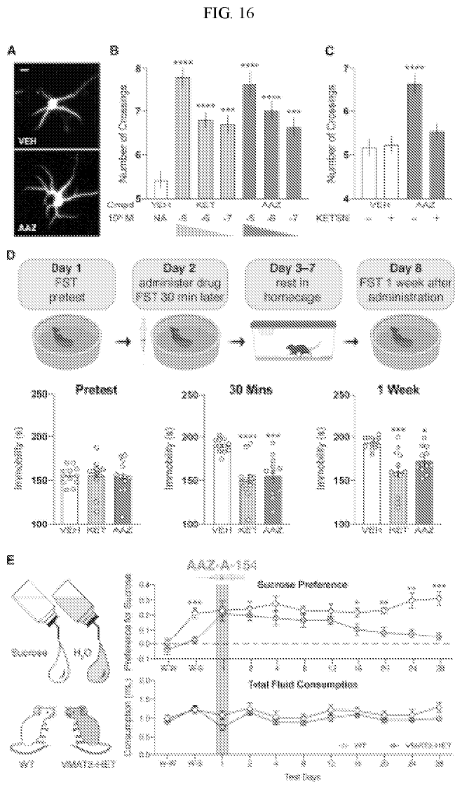

Given its similar structure to several known psychoplastogens (Ly et al., 2018), we tested the ability of AAZ-A-154 to promote dendritic outgrowth in cultured rat embryonic cortical neurons (Cameron et al., 2021; Dunlap et al., 2020). Treatment with AAZ-A-154 increases dendritic arbor complexity to a comparable extent as the fast-acting antidepressant ketamine (KET) (FIGS. 16A and B). This psychoplastogenic effect was abolished by the 5-HT2R antagonist ketanserin (KETSN) (FIG. 16C), suggesting that AAZ-A-154 triggers dendritic growth through activation of 5-HT2Rs.

Hallucinogenic and dissociative psy-choplastogens, are known to produce both rapid and sustained antidepressant effects (Olson, 2018). Because AAZ-A-154 is not predicted to produce hallucinations (FIG. 14E and FIG. 15E), we were interested in assessing its antidepressant potential in vivo using behavioral assays relevant to active stress-coping strategies (i.e., forced swim test) and anhedonia (i.e., sucrose preference). AAZ-A-154 decreased immobility in the forced swim test (FST) (FIG. 16D)—an effortful behavioral response commonly produced by other known psychoplasto-gens (Cameron et al., 2018) and antidepressants such as ketamine (Li et al., 2010). In these studies, we utilized C57BL/6J mice, because this strain does not respond robustly to traditional antidepressants such as selective serotonin reuptake inhibitors (SSRIs) or tricyclics (Has-coot and Bourin, 2009), thus highlighting the similarity between AAZ-A-154 and next-generation antidepressants like ketamine. AAZ-A-154 produced both rapid (30 min) and long-lasting (1 week) antidepressant-like effects after a single administration (FIG. 16D).

To determine if AAZ-A-154 could ameliorate anhedonia, we used VMAT2 heterozygous (VMAT2-HET) mice. We chose this animal model of depression because pharmacological inhibition of VMAT2 precipitates depressive-like behaviors in humans, and VMAT2-HET mice display several depressive phenotypes including a reduced preference for a 1% sucrose solution over water alone (Fukui et al., 2007). At baseline, the wild-type (WT) animals displayed a strong preference for the sucrose solution whereas the VMAT2-HET mice did not (FIG. 16E). However, immediately following a single administration of AAZ-A-154, the VMAT2-HET mice exhibited a sucrose preference that was indistinguishable from WT controls. This anti-anhedonic effect persisted for at least 12 days before the treated VMAT2-HET animals began to display reduced sucrose preference (FIG. 16E). Notably, the change in sucrose preference observed for the VMAT2-HET mice cannot be attributed to differential fluid consumption because both genotypes drank similar volumes of liquids across the entire experiment (FIG. 16E). Moreover, the effects of AAZ-A-154 cannot be ascribed to increasing sucrose palatability, because AAZ-A-154 did not modify sucrose preference in the WT animals FIG. 16E). Taken together, these results suggest that psychLight can be used to identify both hallucinogenic and non-hallucinogenic ligands of the 5-HT2AR.

### DISCUSSION

We developed psychLight as a 5-HT2AR-based fluorescent sensor capable of measuring endogenous 5-HT dynamics and detecting hallucinogenic conformations of the receptor. PsychLight exhibits millisecond off kinetics, which enabled us to detect time-dependent release/reuptake of 5-HT ex vivo and in vivo. Interestingly, we observed both fast and slow decaying 5-HT signals in acute BNST slices following electrical stimulation. However, it is unclear what causes the differential 5-HT time courses, although an SSRI can increase the amplitude of the response and slow reuptake. Compared to iSeroSnFR (Unger et al., 2020), psychLight displayed a much higher apparent affinity even with a relatively smaller dynamic range. These intrinsic properties may make psychLight extremely useful for reporting low concentration events, although psychLight is likely to become fully saturated following a massive release of 5-HT. Together with existing genetically encoded indicators (Unger et al., 2020; Wan et al., 2021), we anticipate that psychLight will prove essential for fully understanding the effects of endogenous 5-HT on brain function. Future side-by-side comparisons of the sensors' properties under identical experimental conditions across various species will provide useful information to guide which sensor to choose for a particular in vivo application.

Unlike existing serotonin sensors, psychLight is based on the 5-HT2AR, which plays an essential role in the hallucinogenic effects of psychedelics. Thus, the fluorescence changes of psy-chLight correlate with ligand-induced conformational changes specific to serotonergic hallucinogens. This is a unique feature of psychLight compared to other 5-HT sensors. In fact, iSeroSnFR exhibits low affinity for many hallucinogenic 5-HT2AR ligands (Unger et al., 2020). In principle, extensive binding pocket engineering of iSeroSnFR could produce a sensor specific for a single hallucinogenic compound, but such a sensor would not be generalizable to the broad class of structurally diverse serotonergic hallucinogens. PsychLight solves this issue by directly measuring conformational changes of the 5-HT2AR—a receptor that is activated by a wide range of diverse serotonergic hallucinogens including tryptamines, ergolines, and amphetamines. This direct measurement of 5-HT2AR conformational change overcomes the limitations of existing methods, which either provide a snap-shot view of the interaction or depend on slow, indirect secondary signaling (González-Maeso et al., 2007). However, to fully understand the action of biased 5-HT2AR ligands at the molecular level, the structures of psy-chLight bound to activating and inactivating ligands will be essential. Determining the spatial and temporal kinetics of ligand-receptor interactions and correlating this information to downstream signaling will provide additional insight into ligands' molecular and cellular mechanisms of action.

PsychLight fills the gap between in vitro testing of novel compounds and in vivo behavioral studies. To date, labor-intensive and costly rodent HTR and DD assays have been the most commonly used methods to assess the hallucinogenic potentials of novel compounds (Halberstadt et al., 2020). González-Maeso et al. (2007) have demonstrated that hallucinogenic and non-hallucinogenic 5-HT2AR ligands induce distinct immediate early gene expression patterns and may differentially activate 5-HT2AR-mGluR2 heterodimers (González-Maeso et al., 2007, 2008). However, these results have yet to be developed into a reliable cellular assay capable of differentiating between hallucinogenic and non-hallucinogenic congeners across a wide range of chemical structures. Using psychLight, hallucinogenic potential can be rapidly assessed in cells through direct fluorescence readout, enabling the identification of potential hallucinogens at an early stage in the drug discovery process. We predict that this assay will be easily adapted to a 384-well format and will complement additional orthogonal GPCR assays (e.g., Ca²⁺ flux, G protein activation, b-arrestin activation, cAMP production, etc.).

PsychLight can be used to identify non-hallucinogenic 5-HT2AR antagonists (e.g., antipsychotics like clozapine) or non-hallucinogenic biased agonists (e.g., LIS). Non-hallucinogenic psychoplastogens have emerged as an incredibly exciting class of 5-HT2AR ligands given the broad implications that neural plasticity-promoting compounds have for treating a variety of brain disorders (Cameron et al., 2021; Dunlap et al., 2020). We used psychLight to identify AAZ-A-154—a non-hallucinogenic analog of a psychedelic compound occupying previously unknown chemical space that promotes neuronal growth and produces long-lasting (>2-week) beneficial behavioral effects in rodents following a single administration. Tabernanthalog (TBG) is the only other known non-hallucinogenic psychoplastogen with antidepressant-like properties (Cameron et al., 2021), and TBG has a similar ligand score as AAZ-A-154 (FIG. 14E). In vivo, it appears that AAZ-A-154 may be more potent than TBG while producing more sustained antidepressant effects.

To date, the precise mechanisms of action of hallucinogens at molecular and circuit levels remain largely unknown (Aghajanian and Marek, 1999; Preller et al., 2018). Genetic tools including reporters, sensors, and effectors that enable the monitoring and manipulation of neuronal activity will be useful for dissecting the circuits involved in hallucinogenic versus antidepressant effects. Furthermore, the identification of functionally selective GPCR ligands will be key to the advancement of future therapeutics targeting this class of receptors. The development of psy-chLight outlines a general strategy for achieving this goal by directly measuring distinct, behaviorally relevant, ligand-induced conformational changes.

Although the foregoing invention has been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

SEQUENCE LISTING

SEQ ID NO: 1-amino acid sequence of enhanced green
fluorescent protein (eGFP)-circular permutation cleavage
motif in seventh beta sheet underlined;
GenBank Accession No. AFA52654.1 GI: 375332228
msrvskgeel ftgvvpilve ldgdvnghkf svsgegegda tygkltlkfi cttgklpvpw
ptlvttlt [symbol] g vqcfsrypdh mkqhdffksa mpegyvqert iffkddgnyk traevkfegd
tlvnrielkg idfkedgnil ghkleynyns hnvyimadkq kngikvnfki rhniedgsvq
ladhyqqntp igdgpvllpd nhyls [symbol] qsal skdpnekrdh mvllefvtaa gitlgmdely
k
Y69W-Substitute tyrosine at position 69 to tryptophan to
create cyan fluorescent protein (CFP)
T206Y-Substitute threonine at position 206 to tyrosine to
create yellow fluorescent protein (YFP)
SEQ ID NO: 2-amino acid sequence of enhanced cyan fluorescent
protein (eCFP)-circular permutation cleavage motif in
seventh beta sheet underlined;
GenBank Accession No. ABN59503.1 GI: 125976354
mvskgeelft gvvpilveld gdvnghkfsv sgegegdaty gkltlkfict tgklpvpwpt
lvttlt [symbol] gvq cfsrypdhmk qhdffksamp egyvqertif fkddgnyktr aevkfegdtl
vnrielkgid fkedgnilgh kleynyishn vyitadkqkn gikanfkirh niedgsvqla
dhyqqntpig dgpvllpdnh ylstqsalsk dpnekrdhmv llefvtaagi tlgmdelyk
SEQ ID NO: 3-amino acid sequence of yellow fluorescent
protein (YFP)-circular permutation cleavage motif in seventh
beta sheet underlined;
GenBank Accession No. AFI26426.1 GI: 384875628
mvskgeelft gvvpilveld gdvnghkfsv sgegegdaty gkltlkfict tgklpvpwpt
lvttfgyglq cfarypdhmk lhdffksamp egyvqertif fkddgnyktr aevkfegdtl
vnrielkgid fkedgnilgh kleynynshn vyimadkqkn gikvnfkirh niedgsvqla
dhyqqntpig dgpvllpdnh yls [symbol] qsalsk dpnekrdhmv llefvtaagi tlgmdelyk
SEQ ID NO: 4-amino acid sequence of Photoactivatable green
fluorescent protein (paGFP) (from Patterson, G. H.;
Lippincott-Schwartz, J. Science 2002, 297, 1873-1877);
circular permutation cleavage motif in seventh beta sheet
underlined;
mvskgeelftgvvpilveldgdvnghkfsvsgegegdatygkltlkficttgklpvpwptlv
ttfsygvqcfsrypdhmkqhdffksampegyvqertiffkddgnyktraevkfegdtlvnri
elkgidfkedgnilghkleynynshnvyimadkqkngikanfkirhniedgsvqladhyqqn
tpigdgpvllpdnhylshqsalskdpnekrdhmvllefvtaagitlgmdelyk
SEQ ID NO: 5-amino acid sequence of superfolder form of the
green fluorescent protein (sfGFP) (from Pedelacq J. D., Nat
Biotechnol 2006 Jan; 24 (1): 79-88); circular permutation
cleavage motif in seventh beta sheet underlined;
mskgeelftgvvpilveldgdvnghkfsvrgegegdatngkltlkficttgklpvpwptlvt
tltygvqcfsrypdhmkrhdffksampegyvqertisfkddgtyktraevkfegdtlvnrie
lkgidfkedgnilghkleynfnshnvyitadkqkngikanfkirhnvedgsvqladhyqqnt
pigdgpvllpdnhylstqsvlskdpnekrdhmvllefvtaagithgmdelyk
SEQ ID NO: 6-amino acid sequence of monomeric cherry red
fluorescent protein (mCherry)-circular permutation cleavage
motif in seventh beta sheet underlined;
GenBank Accession No. ANO45948.1 GI: 1041520567
mvskgeednm aiikefmrfk vhmegsvngh efeiegegeg rpyegtqtak lkvtkggplp
fawdilspqf mygskayvkh padipdylkl sfpegfkwer vmnfedggvv tvtqdsslqd
gefiykvklr gtnfpsdgpv mqkktmgwea ssermypedg alkgeikqrl klkdgghyda
evkttykakk pvqlpgaynv niklditshn edytiveqye raegrhst
SEQ ID NO: 7-amino acid sequence of Photoactivatable
monomeric cherry red fluorescent protein (PAmCherry-1) (from
Suback F.V. et al., Nature Methods 6, 153-159 (2009);
circular permutation cleavage motif in seventh beta sheet
underlined;
mvskgeednmaiikefmrfkvhmegsvnghvfeiegegegrpyegtqtaklkvtkggplpft
wdilspqfmygsnayvkhpadipdyfklsfpegfkwervmkfedggvvtvtqdsslqdgefi
ykvklrgtnfpsdgpvmqkktmgwealsermypedgalkgevkprvklkdgghydaevktty
kakkpvqlpgaynvnrklditshnedytiveqyeraegrhstggmdelyk
SEQ ID NO: 8-amino acid sequence of monomeric red fluorescent
protein (mApple)-circular permutation cleavage motif in
seventh beta sheet underlined;
GenBank Accession No. AEM37572.1 GI: 343458870
mvskgeennm aiikefmrfk vhmegsvngh efeiegegeg rpyeafqtak lkvtkggplp
fawdilspqf mygskvyikh padipdyfkl sfpegfrwer vmnfedggii hvnqdsslqd
gvfiykvklr gtnfpsdgpv mqkktmgwea seermypedg alkseikkrl klkdgghyaa
evkttykakk pvqlpgayiv dikldivshn edytiveqye raegrhstgg mdelyk
SEQ ID NO: 9-amino acid sequence of monomeric red fluorescent
protein (mRuby2)-circular permutation cleavage motif in
seventh beta sheet underlined;
GenBank Accession No. AKE14366.1 GI: 810222672
mvskgeelik enmrmkvvme gsvnghqfkc tgegegnpym gtqtmrikvi eggplpfafd
ilatsfmygs rtfikypkgi pdffkqsfpe gftwervtry edggvvtvmq dtsledgclv
yhvqvrgvnf psngpvmqkk tkgwepntem mypadgglrg ythmalkvdg gghlscsfvt
tyrskktvgn ikmpgihavd hrlerleesd nemfvvqreh avakfaglgg gmdelyk
SEQ ID NO: 10-amino acid sequence Monomeric Far-Red
Fluorescent Protein (mKate2) (from Shcherbo D. et al., Biochem
J. 2009 418 (3): 567-74); circular permutation cleavage motif in
seventh beta sheet underlined;
mvselikenmhmklymegtvnnhhfkctsegegkpyegtqtmrikaveggplpfafdilats
fmygsktfinhtqgipdffkqsfpegftwervttyedggvltatqdtslqdgcliynvkirg
vnfpsngpvmqkktlgweastetlypadgglegradmalklvggghlicnlkttyrskkpak
nlkmpgvyyvdrrlerikeadketyveqhevavarycdlpsklghr
SEQ ID NO: 11-amino acid sequence of green-to-red
photoconvertible fluorescent protein mEos2-GenBank Accession
No. ACN37844.1 GI: 224037249; circular permutation cleavage
motif in seventh beta sheet underlined;
msaikpdmki klrmegnvng hhfvidgdgt gkpfegkqsm dlevkeggpl pfafdiltta
fhygnrvfak ypdnigdyfk qsfpkgyswe rsltfedggi ciarnditme gdtfynkvrf
ygtnfpangp vmqkktlkwe pstekmyvrd gvltgdihma lllegnahyr cdfrttykak
ekgvklpgyh fvdhcieils hdkdynkvkl yehavahsgl pdnarr
SEQ ID NO: 12-amino acid sequence of mMaple; from McEvoy, et
al., PLOS One. 2012; 7 (12): e51314; circular permutation
cleavage motif in seventh beta sheet underlined;
mvskgeetimsvikpdmkiklrmegnvnghafviegegsgkpfegiqtidlevkegaplpfa
ydilttafhygnrvftkypedipdyfkqsfpegyswersmtyedggiciatnditmeedsfi
nkihfkgtnfppngpvmqkrtvgwevstekmyvrdgvlkgdvkmklllkggshyrcdfrtty
kvkqkavklpdyhfvdhrieilshdkdynkvklyehavarnstdsmdelyk
SEQ ID NO: 13-amino acid sequence of far-red fluorescent
protein mCardinal; GenBank: AHL19967.1, from Chu, et al., Nat.
Methods (2014) 11 (5): 572-578; circular permutation cleavage
motif in seventh beta sheet underlined;
mvskgeelik enmhmklyme gtvnnhhfkc ttegegkpye gtqtqrikvv eggplpfafd
ilatcfmygs ktfinhtqgi pdffkqsfpe gftwervtty edggvltvtq dtslqdgcli
ynvklrgvnf psngpvmqkk tlgweattet lypadggleg rcdmalklvg gghlhcnlkt
tyrskkpakn lkmpgvyfvd rrlerikead netyveqhev avarycdlps klghklngmd
elyk
SEQ ID NO: 14-amino acid sequence of mNeptune; GenBank:
CBH32884.1, from Lin, et al., Chem. Biol. (2009) 16(11): 1169-
1179; circular permutation cleavage motif in seventh beta
sheet underlined;
mvskgeelik enmhmklyme gtvnnhhfkc tsegegkpye gtqtgrikvv eggplpfafd
ilatcfmygs ktfinhtqgi pdffkqsfpe gftwervtty edggvltatq dtslqdgcli
ynvkirgvnf psngpvmqkk tlgweastet lypadggleg rcdmalklvg gghlicnlkt
tyrskkpakn lkmpgvyfvd rrlerikead netyveqhev avarycdlps klghklngmd
elyk
SEQ ID NO: 15-amino acid sequence of circularly permuted
green fluorescent protein; N-terminus at residue 148 in eGFP
(SEQ ID NO: 49) ; GGTGGS linker connecting C-terminus to N
terminus underlined;
n v y i k a d k q k n g i k a n f k i r h n i e d g g v q l a
y h y q q n t p i g d g p v l l p d n h y l s [symbol] q s k l s k d
p n e k r d h m v l l e f v t a a g i t l g m d e l y k g g t
g g s m v s k g e e l f t g v v p i l v e l d g d v n g h k f
s v s g e g e g d a t [symbol] g k l t l k f i c t t g k l p v p w p
t l v t t l t y g v q c f s r y p d h m k q h d f f k s a m p
e g y i q e r t i f f k d d g n y k
V55Y:
Substitute threonine at position 55 to tyrosine to
create yellow fluorescent protein (cpYFP)
Y136W: Substitute tyrosine at position 136 to tryptophan to
create cyan fluorescent protein (cpCFP)
SEQ ID NO: 16-amino acid sequence of circularly permuted
mRuby2; N-terminus at residue 147 in mRuby2 (SEQ ID NO: 9);
GGTGGS linker connecting C-terminus to N terminus underlined
(from Akerboom J et al., Front Mol Neurosci., 2013 6:2)
NTEMMYPADGGLRGYTHMALKVDGGGHLSCSFVTTYRSKKTVGNIKMPGIHYVSHRLERLEE
SDNEMFVVQREHAVAKFVGLGGGGGTGGSMNSLIKENMRMKVVLEGSVNGHQFKCTGEGEGN
PYMGTQTMRIKVIEGGPLPFAFDILATSFMSRTFIKYPKGIPDFFKQSFPEGFTWERVTRYE
DGGVITVMQDTSLEDGCLVYHAQVRGVNFPSNGAVMQKKTKGWEPTRDQLT
SEQ ID NO: 17-amino acid sequence of circularly permuted
mApple; GGTGGS linker connecting C-terminus to N terminus
underlined (from Zhao Y. et al., Science, 2011 333 (6051):
1888-1891)
VVSERMYPEDGALKSEIKKGLRLKDGGHYAAEVKTTYKAKKPVQLPGAYIVDIKLDIVSHNE
DYTIVEQCERAEGRHSTGGMDELYKGGTGGSLVSKGEEDNMAIIKEFMRFKVHMEGSVNGHE
FEIEGEGEGRPYEAFQTAKLKVTKGGPLPFAWDILSPQFMSKAYIKHPADIPDYFKLSFPEG
FRWERVMNFEDGGIIHVNQDSSLQDGVFIYKVKLRGTNFPPDGPVMQKKTMGWEA
SEQ ID NO: 18-amino acid sequence of circularly permuted
mEos2; GGTGGS linker connecting C-terminus to N terminus
underlined (from Fosque B.F. et al., Science 2015
347 (6223): 755-60)
LECEKMYVRDGVLTGDIHMALLLEGNAHYRCDFRTTYKAKEKGVKLPGYHFVDHCIEILSHD
KDYNKVKLYEHAVAHSGLPDNARRGGTGGSMVSAIKPDMKIKLRMEGNVNGHHFVIDGDGTG
KPYEGKQTMDLEVKEGGPLPFAFDILTTAFHNRVFVKYPDNIQDYFKQSFPKGYSWERSMTF
EDGGICYARNDITMEGDTFYNKVRFYGTNFPANGPVMQKKTLKW
SEQ ID NO: 19-motif in beta strand seven of fluorescent
protein (e.g., green fluorescent protein, cyan fluorescent
protein, yellow fluorescent protein)
YN(Y/F)(N/I)SHNV
SEQ ID NO: 20-motif in beta strand seven of a red fluorescent
protein (e.g., mCherry, mRuby2, mRuby3, mApple, mKate2, mEos2,
mMaple, mCardinal, mNeptune)
WE(A/P/V)(S/L/N/T)(S/E/T)E(R/M/T/K)(M/L)
SEQ ID NO: 21-nucleic acid sequence of GFP sensor integrated
into third intracellular loop of beta2 adrenergic receptor
atgaagacgatcatcgccctgagctacatcttctgcctggtgttcgccgactacaaggacga
tgatgacgccatggggcagccaggtaatggctctgcgttcttgttggccccgaacaggagcc
atgctcccgaccatgacgtcacccaacagagagatgaggtctgggtagtaggcatgggtatt
gtcatgtctctgatagtcttggcaatcgtgtttggaaatgtgctcgttatcacggcaatagc
taagtttgagcgacttcaaacggtaacaaattatttcataacatctctcgcgtgtgcagatc
tcgtaatgggactcgctgtggtcccctttggcgcggcccatatcctgatgaagatgtggact
tttggtaatttctggtgtgaattttggaccagcatagatgtactctgtgttacagcttcaat
tgaaactctctgtgtgatagccgttgatcgctatttcgccattacgtcccct [symbol] aagtatc
aatcattgcttaccaagaataaagcccgagtaataattctcatggtgtggatcgtaagcggg
ctcacatcttttttgccgattcagatgcactggtatagagcaacgcaccaagaagccataaa
ctgctacgcaaatgaaacttgctgtgacttctttacaaatcaggcttacgctattgcctctt
caatagtcagtttttacgttcctctggttattatggtgtttgtatactcacgggtattccag
gaggctaagcggcagctccagaaaatagacaagagtgagggacgctttcatgtgcagaatct
ttcaGCCGTCAACGTCTATATCAAGGCCGACAAGCAGAAGAACGGCATCAAGGCGAACTTCA
AGATCCGCCACAACATCGAGGACGGCGGCGTGCAGCTCGCCTACCACTACCAGCAGAACACC
CCCATCGGCGACGGCCCCGTGCTGCTGCCCGACAACCACTACCTGAGCGTGCAGTCCAAACT
TTCGAAAGACCCCAACGAGAAGCGCGATCACATGGTCCTGCTGGAGTTCGTGACCGCCGCCG
GGATCACTCTCGGCATGGACGAGCTGTACAAGGGCGGTACCGGAGGGAGCATGGTGAGCAAG
GGCGAGGAGCTGTTCACCGGGGTGGTGCCCATCCTGGTCGAGCTGGACGGCGACGTAAACGG
CCACAAGTTCAGCGTGTCCGGCGAGGGTGAGGGCGATGCCACCTACGGCAAGCTGACCCTGA
AGTTCATCTGCACCACCGGCAAGCTGCCCGTGCCCTGGCCCACCCTCGTGACCACCCTGACC
TACGGCGTGCAGTGCTTCAGCCGCTACCCCGACCACATGAAGCAGCACGACTTCTTCAAGTC
CGCCATGCCCGAAGGCTACATCCAGGAGCGCACCATCTTCTTCAAGGACGACGGCAACTACA
AGACCCGCgccgaggtgaagttcgagggcgacaccctggtgaaccgcatcgagctgaagggc
atcgacttcaaggaggacggcaacatcctggggcacaagctggagtacaacaccagacaagt
tgaacaggacggacgcacaggtcatggcctcaggaggagttctaagttctgcttgaaggagc
acaaagcgctgaagacgcttggaattatcatggggacgtttactctctgctggcttcctttc
ttcatagtaaacattgttcacgtaatccaagacaatctgattcgaaaggaggtgtatattct
cctcaattggattgggtacgtaaacagcggatttaatcctcttatctattgccgaagccctg
atttccgcatagcctttcaggaactgctttgtcttcgccgaagcagccttaaagcgtacgga
aatggttac [symbol] aatgggaatacaggcgagcaaagcgggtatcacgtcgagcaagagaa
ggagaacaaacttctgtgcgaagacctgcctggcacggaagattttgtcggacaccaaggga
cggtaccgagtgacaatatcgacagtcaaggccgaaactgctcaactaatgattcactcctg
tag

Feature Annotations for SEQ ID NO: 21

| Residue | | |
|---|---|---|
| numbers | Formatting | Feature definition |
| 1-45 | Underlined | Secretory sequence (from |
| | | Hemagglutinin) |
| 46-72 | Italics | Flag-tag (non essential to sensor |
| | | function) |
| 487-489 | Bold and | Mutations from wild-type B2AR |
| and | double | sequence to generate signaling- |
| 1870-1875 | underline | incompetent sensor; F139A and SS355- |
| | | 356AA |
| 811-1434 | CAPITALS | circularly permuted GFP (cpGFP) |
| | | (including nucleic acids encoding |
| | | alanine-valine (AV) linker at 3' end |
| | | and threonine-arginine (TR) linker |
| | | at 5' end of cpGFP) |
| 811-816 | ITALIC | alanine-valine (AV) linker at 3' end |
| and 1429- | CAPITALS | and threonine-arginine (TR) linker |
| 1434 | | at 5' end of cpGFP |

-amino acid sequence of GFP sensor integrated
into third intracellular loop of beta2 adrenergic receptor
SEQ ID NO: 22
m k t i i a l s y i f c l v f a d y k d d d d a m g q p g n g
s a f l l a p n r s h a p d h d v t q q r d e v w v v g m g i
v m s l i v l a i v f g n v l v i t a i a k f e r l q t v t n
y f i t s l a c a d l v m g l a v v p f g a a h i l m k m w t
f g n f w c e f w t s i d v l c v t a s i e t l c v i a v d r
y f a i t s p [symbol] k y q s l l t k n k a r v i i l m v w i v s g
l t s f l p i q m h w y r a t h q e a i n c y a n e t c c d f
f t n q a y a i a s s i v s f y v p l v i m v f v y s r v f q
e a k r q l q k i d k s e g r f h v q n l s A V N V Y I K A D
K Q K N G I K A N F K I R H N I E D G G V Q L A Y H Y Q Q N T
P I G D G P V L L P D N H Y L S V Q S K L S K D P N E K R D H
M V L L E F V T A A G I T L G M D E L Y K G G T G G S M V S K
G E E L F T G V V P I L V E L D G D V N G H K F S V S G E G E
G D A T Y G K L T L K F I C T T G K L P V P W P T L V T T L T
Y G V Q C F S R Y P D H M K Q H D F F K S A M P E G Y I Q E R
T I F F K D D G N Y K T R a e v k f e g d t l v n r i e l k g
i d f k e d g n i l g h k l e y n t r q v e q d g r t g h g l
r r s s k f c l k e h k a l k t l g i i m g t f t l c w l p f
f i v n i v h v i q d n l i r k e v y i l l n w i g y v n s g
f n p l i y c r s p d f r i a f q e l l c l r r s s l k a y g
n g y [symbol] n g n t g e q s g y h v e q e k e n k l l c e d l p
g t e d f v g h q g t v p s d n i d s q g r n c s t n d s l l
stop

Feature Annotations for SEQ ID NO: 22

| Residue | | |
|---|---|---|
| numbers | Formatting | Feature definition |
| 1-15 | Underlined | Secretory sequence (from |
| | | Hemagglutinin) |
| 16-24 | Italics | Flag-tag (non essential to sensor |
| | | function) |
| 163 | Bold and | X is any amino acid or an amino acid |
| | double | selected from the group consisting |
| | underline | of F, A, G, V, I, L, M, S, T |
| 163 and | Bold and | Mutations from wild-type B2AR |
| 624-625 | double | sequence to generate signaling- |
| | underline | incompetent sensor; F139A and SS355- |
| | | 356AA |
| 248-270 | Underline | Third intracellular loop, split by |
| and | and italics | integration of sensor |
| 479-538 | | |
| 271-478 | CAPITALS | circularly permuted GFP (cpGFP) |
| | | (including alanine-valine (AV) |
| | | linker at N-terminus and threonine- |
| | | arginine (TR) linker at C-terminus |
| | | of cpGFP) |
| 271-272 | ITALIC | alanine-valine (AV) linker at N- |
| and 477- | CAPITALS | terminus and threonine-arginine (TR) |
| 478 | | linker at C-terminus of cpGFP |

-nucleic acid sequence of GFP sensor integrated
into third intracellular loop of u-type opioid receptor
SEQ ID NO: 23
atgaagacgatcatcgccctgagctacatcttctgcctggtgttcgccgactacaaggacga
tgatgacgccatggatagtagcgctgcgcctaccaacgcgtcaaactgcaccgatgctcttg
cgtactcctcctgctccccggcacctagtcccggttcttgggtcaatttgtcccatctggac
ggaaacctctctgatccctgtgggcctaacaggacggacctcggtgggagggactccctttg
cccgccgaccggatctccgtccatgataacggccattacaattatggcgttgtatagcatcg
tatgcgttgtaggtctttttgggaatttcctggtgatgtacgtcatcgtcaggtacacaaag
atgaaaacagctactaacatttatatatttaacctggcgctcgcggacgctctcgcaacgtc
aacgctcccgtttcagtccgtgaattatctcatgggtacttggcctttcggaacaatactgt
gtaaaattgttataagcatagattattataatatgttcacgtccatcttcacactctgcaca
atgtctgtggataggtacattgctgtatgtcaccca [symbol] aaggcgcttgactttagaactcc
acgcaatgcaaagattataaatgtgtgcaactggatcttgtcctctgcaatagggcttcctg
tgatgttcatggcgactactaagtacagacagggcagcatagattgcacactcaccttctca
cacccaacttggtactgggaaaatctgctcaagatctgcgtcttcatttttgcttttatcat
gccagtattgataatcacggtctgttacgggttgatgattttgcggctcaaatcagttcgaa
tgctcagtATCAAAAACGTCTATATCAAGGCCGACAAGCAGAAGAACGGCATCAAGGCGAAC
TTCAAGATCCGCCACAACATCGAGGACGGCGGCGTGCAGCTCGCCTACCACTACCAGCAGAA
CACCCCCATCGGCGACGGCCCCGTGCTGCTGCCCGACAACCACTACCTGAGCGTGCAGTCCA
AACTTTCGAAAGACCCCAACGAGAAGCGCGATCACATGGTCCTGCTGGAGTTCGTGACCGCC
GCCGGGATCACTCTCGGCATGGACGAGCTGTACAAGGGCGGTACCGGAGGGAGCATGGTGAG
CAAGGGCGAGGAGCTGTTCACCGGGGTGGTGCCCATCCTGGTCGAGCTGGACGGCGACGTAA
ACGGCCACAAGTTCAGCGTGTCCGGCGAGGGTGAGGGCGATGCCACCTACGGCAAGCTGACC
CTGAAGTTCATCTGCACCACCGGCAAGCTGCCCGTGCCCTGGCCCACCCTCGTGACCACCCT
GACCTACGGCGTGCAGTGCTTCAGCCGCTACCCCGACCACATGAAGCAGCACGACTTCTTCA
AGTCCGCCATGCCCGAAGGCTACATCCAGGAGCGCACCATCTTCTTCAAGGACGACGGCAAC
TACAAGACCCGCGCCGAGGTGAAGTTCGAGGGCGACACCCTGGTGAACCGCATCGAGCTGAA
GGGCATCGACTTCAAGGAGGACGGCAACATCCTGGGGCACAAGCTGGAGTACAACATTATCg
gcagcaaggagaaggaccgcaacctcagaaggataacgagaatggtgctggtcgtagtggcg
gttttcattgtttgttggacgccaatacacatatacgtgattataaaggctctggtgacaat
tcccgaaacaacgtttcagacggtctcttggcatttctgtattgcattggggtacactaatt
cctgccttaatcctgtattgtacgcctttctggatgaaaactttaaaagatgtttccgcgag
ttctgcataccgaccagcagcaacattgaacaacaaaactccacgcgcatacggcaaaatac
tagggatcacccgtccactgcgaatactgtagaccgaacgaaccatcagttggagaatttgg
aagcggaaactgctcctctgccatga

Feature Annotations for SEQ ID NO: 23

| Residue | | |
|---|---|---|
| numbers | Formatting | Feature definition |
| 1-45 | Underlined | Secretory sequence (from |
| | | Hemagglutinin) |
| 46-72 | Italics | Flag-tag (non essential to sensor |
| | | function) |
| 595-597 | Bold and | V199A mutation from wild-type μ-type |
| | double | opioid receptor signaling |
| | underline | incompetent |
| 877-1611 | CAPITALS | circularly permuted GFP (cpGFP) |
| | | (including nucleic acids encoding |
| | | isoleucine-lysine (IK) linker at 3' |
| | | end and isoleucine-isoleucine (II) |
| | | linker at 5' end of cpGFP) |
| 877-882 | ITALIC | isoleucine-lysine (IK) linker at 3' |
| and 1606- | CAPITALS | end and isoleucine-isoleucine (II) |
| 1611 | | linker at 5' end of cpGFP |

amino acid sequence of GFP sensor integrated
into third intracellular loop of u-type opioid
receptor
SEQ ID NO: 24
mktiialsyifclvfadykddddamdssaaptnasnctdalaysscsp
apspgswvnlshldgnlsdpcgpnrtdlggrdslcpptgspsmitait
imalysivcvvglfgnflvmyvivrytkmktatniyifnlaladalat
stlpfqsvnylmgtwpfgtilckivisidyynmftsiftlctmsvdry
iavchp [symbol] kaldfrtprnakiinvonwilssaiglpvmfmattkyrqgs
idctltfshptwywenllkicvfifafimpvliitvcyglmilrlksv
rmlsIKNVYIKADKQKNGIKANFKIRHNIEDGGVQLAYHYQQNTPIGD
GPVLLPDNHYLSVQSKLSKDPNEKRDHMVLLEFVTAAGITLGMDELYK
GGTGGSMVSKGEELFTGVVPILVELDGDVNGHKFSVSGEGEGDATYGK
LTLKFICTTGKLPVPWPTLVTTLTYGVQCFSRYPDHMKQHDFFKSAMP
EGYIQERTIFFKDDGNYKTRAEVKFEGDTLVNRIELKGIDFKEDGNIL
GHKLEYNIIgskekdrnlrritrmvlvvvavfivcwtpihiyviikal
vtipettfqtvswhfcialgytnsclnpvlyafldenfkrcfrefcip
tssnieqqnstrirqntrdhpstantvdrtnhqlenleaetaplp-

Feature Annotations for SEQ ID NO: 24

| Residue | | |
|---|---|---|
| numbers | Formatting | Feature definition |
| 1-15 | Underlined | Secretory sequence |
| | | (from Hemagglutinin) |
| 16-24 | Italics | Flag-tag (non essential to sensor |
| | | function) |
| 199 | Bold and | X is an amino acid or an amino acid selected |
| | double | from the group consisting of |
| | underline | F, A, G, V, I, L, M, S, T |
| 289-292 | Underline | Third intracellular loop, split by |
| and | and italics | integration of sensor |
| 538-539 | | |
| 293-537 | CAPITALS | circularly permuted GFP (cpGFP) |
| | | (including isoleucine-lysine (IK) |
| | | linker at N-terminus and isoleucine- |
| | | isoleucine (II) linker at C-terminus |
| | | of cpGFP) |
| 293-294 | ITALIC | isoleucine-lysine (IK) linker at |
| and | CAPITALS | N-terminus and isoleucine-isoleucine |
| 536-537 | | (II) linker at C-terminus of cpGFP |

nucleic acid sequence of GFP sensor integrated
into third intracellular loop of dopamine
receptor D1 (DRD1)
SEQ ID NO: 25
atgaagacgatcatcgccctgagctacatcttctgcctggtgttcgcc
gactacaaggacgatgatgacgccatgaggactctgaacacctctgcc
atggacgggactgggctggtggtggagagggacttctctgttcgtatc
ctcactgcctgtttcctgtcgctgctcatcctgtccacgctcctgggg
aacacgctggtctgtgctgccgttatcaggttccgacacctgcggtcc
aaggtgaccaacttctttgtcatctccttggctgtgtcagatctcttg
gtggccgtcctggtcatgccctggaaggcagtggctgagattgctggc
ttctggccctttgggtccttctgtaacatctgggtggcctttgacatc
atgtgctccactgcatccatcctcaacctctgtgtgatcagcgtggac
aggtattgggctatctccagccct [symbol] cggtatgagagaaagatgacc
cccaaggcagccttcatcctgatcagtgtggcatggaccttgtctgta
ctcatctccttcatcccagtgcagctcagctggcacaaggcaaaaccc
acaagcccctctgatggaaatgccacttccctggctgagaccatagac
aactgtgactccagcctcagcaggacatatgccatctcatcctctgta
atcagcttttacatccctgtggccatcatgattgtcacctacaccagg
atctacaggattgctcagaaacaaatacggcgcattgcggccttggag
agggcagcagtccacgccaagaattgcTCTCGGAACGTGTATATCAAG
GCTGATAAACAAAAGAATGGTATCAAAGCTAATTTCAAAATCCGCCAC
AATATCGAAGATGGCGGCGTCCAGCTCGCTTATCATTATCAGCAGAAT
ACACCTATCGGTGACGGGCCGGTGCTTTTGCCTGATAACCATTACCTG
AGTGTTCAAAGTAAACTGAGCAAGGATCCAAATGAAAAAAGGGACCAC
ATGGTGCTTCTCGAATTTGTAACGGCTGCAGGCATTACTCTCGGGATG
GACGAACTTTACAAAGGAGGGACCGGAGGCAGCATGGTGTCCAAGGGG
GAGGAACTTTTCACTGGCGTCGTGCCGATACTCGTCGAACTCGATGGA
GATGTTAATGGACACAAATTTTCAGTCAGTGGCGAAGGGGAAGGGGAT
GCTACTTACGGGAAACTCACACTGAAGTTTATTTGTACGACAGGCAAA
CTCCCGGTACCTTGGCCGACCTTGGTGACCACGTTGACGTATGGAGTA
CAGTGCTTCTCCAGGTACCCGGACCACATGAAGCAACATGACTTTTTC
AAAAGCGCTATGCCCGAGGGCTACATTCAAGAACGGACTATTTTCTTT
AAGGACGATGGAAACTATAAAACCAGAGCTGAGGTGAAATTCGAGGGT
GACACTCTTGTAAACCGGATAGAACTCAAAGGTATAGATTTCAAAGAA
GACGGAAACATCTTGGGGCATAAACTCGAGTATAATCCTCCTcagacc
accacaggtaatggaaagcctgtcgaatgttctcaaccggaaagttct
tttaagatgtccttcaaaagagaaactaaagtcctgaagactctgtcg
gtgatcatgggtgtgtttgtgtgctgttggctacctttcttcatcttg
aactgcattttgcccttctgtgggtctggggagacgcagcccttctgc
attgattccaacacctttgacgtgtttgtgtggtttgggtgggctaat
tcatccttgaaccccatcatttatgcctttaatgctgattttcggaag
gcattttcaaccctcttaggatgctacagactttgccctgcgacgaat
aatgccatagagacggtgagtatcaataacaatggggccgcgatgttt
tccagccatcatgagccattctgctacgagaatgaagtctga

Feature Annotations for SEQ ID NO: 25

| Residue | | |
|---|---|---|
| numbers | Formatting | Feature definition |
| 1-45 | Underlined | Secretory sequence |
| | | (from Hemagglutinin) |
| 46-72 | Italics | Flag-tag (non essential to sensor |
| | | function) |
| 457-459 | Bold and | F129A mutation from wild-type DRD1 |
| | double | sequence to generate signaling- |
| | underline | incompetent sensor |
| 796-1530 | CAPITALS | circularly permuted GFP (cpGFP) |
| | | (including nucleic acids encoding |
| | | serine-arginine (SR) linker at 3′ |
| | | end and proline-proline (PP) linker |
| | | at 5′ end of cpGFP) |
| 796-801 | ITALIC | serine-arginine (SR) linker at 3′ |
| and | CAPITALS | end and proline-proline (PP) linker |
| 1525-1530 | | at 5′ end of cpGFP |
| 1939-1959 | italic and | Endoplasmic Reticulum Export |
| | underlined | Sequence |

amino acid sequence of GFP sensor integrated
into third intracellular loop of dopamine
receptor D1 (DRD1)
SEQ ID NO: 26
mktiialsyifclvfadykddddamrtlntsamdgtglvverdfsvri
ltacflsllilstllgntlvcaavirfrhlrskvtnffvislavsdll
vavlvmpwkavaeiagfwpfgsfcniwvafdimcstasilnlcvisvd
rywaissp [symbol] ryerkmtpkaafilisvawtlsvlisfipvqlswhkakp
tspsdgnatslaetidncdsslsrtyaisssvisfyipvaimivtytr
iyriaqkqirriaaleraavhakncSRNVYIKADKQKNGIKANFKIRH
NIEDGGVQLAYHYQQNTPIGDGPVLLPDNHYLSVQSKLSKDPNEKRDH
MVLLEFVTAAGITLGMDELYKGGTGGSMVSKGEELFTGVVPILVELDG
DVNGHKFSVSGEGEGDATYGKLTLKFICTTGKLPVPWPTLVTTLTYGV
QCFSRYPDHMKQHDFFKSAMPEGYIQERTIFFKDDGNYKTRAEVKFEG
DTLVNRIELKGIDFKEDGNILGHKLEYNPPqtttgngkpvecsqpess
fkmsfkretkvlktlsvimgvfvccwlpffilncilpfcgsgetqpfc
idsntfdvfvwfgwansslnpiiyafnadfrkafstllgcyrlcpatn
naietvsinnngaamfsshhepfcyenev-

Feature Annotations for SEQ ID NO: 26

| Residue | | |
|---|---|---|
| numbers | Formatting | Feature definition |
| 1-15 | Underlined | Secretory sequence |
| | | (from Hemagglutinin) |
| 16-24 | Italics | Flag-tag (non essential to sensor |
| | | function) |
| 153 | Bold and | X is an amino acid or an amino acid selected |
| | double | from the group consisting of |
| | underline | F, A, G, V, I, L, M, S, T |
| 246-265 | italic and | Third intracellular loop, split by |
| and | underlined | integration of sensor |
| 511-538 | | |
| 266-510 | CAPITALS | circularly permuted GFP (cpGFP) |
| | | (including serine-arginine (SR) |
| | | linker at N-terminus and proline- |
| | | proline (PP) linker at C-terminus of |
| | | cpGFP) |
| 266-267 | ITALIC | serine-arginine (SR) linker at N- |
| and | CAPITALS | terminus and proline-proline (PP) |
| 509-510 | | linker at C-terminus of cpGFP |
| 647-653 | italic and | Endoplasmic Reticulum Export |
| | underlined | Sequence |

nucleic acid sequence of GFP sensor integrated
into third intracellular loop of
5-Hydroxytryptamine 2A (5-HT2A) receptor
SEQ ID NO: 27
atgaagacgatcatcgccctgagctacatcttctgcctggtgttcgcc
gactacaaggacgatgatgacgccatggacatactttgtgaagagaat
acttcactctcttctactactaactctcttatgcaactgaacgatgat
acccgattgtactcaaacgacttcaattccggcgaagcgaacaccagt
gacgcattcaactggactgtcgattctgaaaacagaactaatctgtca
tgcgagggttgtcttagtccctcttgtctcagcctgttgcacctccag
gaaaagaactggtcagcactgctcactgcggtagtgataatactcact
attgctggcaatattctcgtaattatggcagtctccttggagaagaaa
ctccaaaacgccacaaattattttcttatgagccttgccatcgcagat
atgctcttgggatttttggtgatgcctgtgagtatgctcacgatactg
tatggatatcgctggcctctgccgtctaaactttgcgctgtgtggatt
tacttggatgtccttttttcaactgcgagtattatgcatctttgcgcc
attagtcttgataggtatgtggctatccaaaatcct [symbol] caccattcc
cgctttaatagtagaactaaggcttttctgaaaataatagcagtgtgg
accatatctgtcggcataagcatgcctatccccgtatttggacttcaa
gatgactcaaaggtattcaaagaagggtcatgtctgctggccgatgac
aatttcgtgcttattggatccttcgtcagtttcttcattcctttgaca
atcatggtgattacctactttcttacgattaaatctttgcaaaaggag
gctactctgtgcgtcagcgacctcggcactcgggccaaaTCTCGGAAC
GTCTATATCAAGGCCGACAAGCAGAAGAACGGCATCAAGGCGAACTTC
AAGATCCGCCACAACATCGAGGACGGCGGCGTGCAGCTCGCCTACCAC
TACCAGCAGAACACCCCCATCGGCGACGGCCCCGTGCTGCTGCCCGAC
AACCACTACCTGAGCGTGCAGTCCAAACTTTCGAAAGACCCCAACGAG
AAGCGCGATCACATGGTCCTGCTGGAGTTCGTGACCGCCGCCGGGATC
ACTCTCGGCATGGACGAGCTGTACAAGGGCGGTACCGGAGGGAGCATG
GTGAGCAAGGGCGAGGAGCTGTTCACCGGGGTGGTGCCCATCCTGGTC
GAGCTGGACGGCGACGTAAACGGCCACAAGTTCAGCGTGTCCGGCGAG
GGTGAGGGCGATGCCACCTACGGCAAGCTGACCCTGAAGTTCATCTGC
ACCACCGGCAAGCTGCCCGTGCCCTGGCCCACCCTCGTGACCACCCTG
ACCTACGGCGTGCAGTGCTTCAGCCGCTACCCCGACCACATGAAGCAG
CACGACTTCTTCAAGTCCGCCATGCCCGAAGGCTACATCCAGGAGCGC
ACCATCTTCTTCAAGGACGACGGCAACTACAAGACCCGCGCCGAGGTG
AAGTTCGAGGGCGACACCCTGGTGAACCGCATCGAGCTGAAGGGCATC
GACTTCAAGGAGGACGGCAACATCCTGGGGCACAAGCTGGAGTACAAC
CTTTTCcttgccagcttctcattccttccccagtcctctctttccagt
gagaaacttttccaacgatccatacatagggagccgggtagttataca
ggacggcggacgatgcaatcaattagtaatgagcaaaaggcttgtaag
gtactcggcatagtcttctttctgtttgtggtgatgtggtgtcccttc
tttataacgaatatcatggcagtgatctgcaaggaatcatgcaatgag
gatgtgatcggggcacttctgaacgttttcgtgtggatagggtatctg
tcaagtgctgtgaacccactggtctataccttgtttaataagacatac
cgctcagccttttcacggtatattcaatgtcagtataaggaaaacaag
aaacctctgcaacttattcttgtgaacactatccctgccctggcttat
aagtcatcacagttgcagatgggccagaaaaaaaattccaagcaggac
gcgaagacaacagacaacgattgtagtatggttgccctcggcaagcag
cacagtgaagaagcgagcaaagacaatagtgatggcgtaaacgaaaaa
gtcagttgtgtataa

Feature Annotations for SEQ ID NO: 27

| Residue | | |
|---|---|---|
| numbers | Formatting | Feature definition |
| 1-45 | Underlined | Secretory sequence |
| | | (from Hemagglutinin) |
| 46-72 | Italics | Flag-tag (non essential to sensor |
| | | function) |
| 613-615 | Bold and | I181A mutation from wild-type 5HT-2A |
| | double | sequence to generate signaling- |
| | underline | incompetent sensor |
| 904-1638 | CAPITALS | circularly permuted GFP (cpGFP) |
| | | (including nucleic acids encoding |
| | | serine-arginine (SR) linker at 3′ |
| | | end and leucine-phenylalanine (LF) |
| | | linker at 5′ end of cpGFP) |
| 904-909 | ITALIC | serine-arginine (SR) linker at 3′ |
| and | CAPITALS | end and leucine-phenylalanine (LF) |
| 1633-1638 | | linker at 5′ end of cpGFP |

amino acid sequence of GFP sensor integrated
into third intracellular loop of
5-Hydroxytryptamine 2A (5-HT2A) receptor
SEQ ID NO: 28
mktiialsyifclvfadykddddamdilceentslssttnslmqlndd
trlysndfnsgeantsdafnwtvdsenrtnlscegclspsclsllhlq
eknwsalltavviiltiagnilvimavslekklqnatnyflmslaiad
mllgflvmpvsmltilygyrwplpsklcavwiyldvlfstasimhlca
isldryvaiqnp [symbol] hhsrfnsrtkaflkiiavwtisvgismpipvfglq
ddskvfkegsclladdnfvligsfvsffipltimvityfltikslqke
atlcvsdlgtrakSRNVYIKADKQKNGIKANFKIRHNIEDGGVQLAYH
YQQNTPIGDGPVLLPDNHYLSVQSKLSKDPNEKRDHMVLLEFVTAAGI
TLGMDELYKGGTGGSMVSKGEELFTGVVPILVELDGDVNGHKFSVSGE
GEGDATYGKLTLKFICTTGKLPVPWPTLVTTLTYGVQCFSRYPDHMKQ
HDFFKSAMPEGYIQERTIFFKDDGNYKTRAEVKFEGDTLVNRIELKGI
DFKEDGNILGHKLEYNLFlasfsflpqsslsseklfqrsihrepgsyt
grrtmqsisneqkackvlgivfflfvvmwcpffitnimavickescne
dvigallnvfvwigylssavnplvytlfnktyrsafsryiqcqykenk
kplqlilvntipalaykssqlqmgqkknskqdakttdndcsmvalgkq
hseeaskdnsdgvnekvscv-

Feature Annotations for SEQ ID NO: 28

| Residue | | |
|---|---|---|
| numbers | Formatting | Feature definition |
| 1-15 | Underlined | Secretory sequence |
| | | (from Hemagglutinin) |
| 16-24 | Italics | Flag-tag (non essential to sensor |
| | | function) |
| 205 | Bold and | X is any amino acid or an amino acid |
| | double | selected from the group consisting of |
| | underline | F, A, G, V, I, L, M, S, T |
| 286-301 | italic and | Third intracellular loop, split by |
| and | underlined | integration of sensor |
| 547-589 | | |
| 302-546 | CAPITALS | circularly permuted GFP (cpGFP) |
| | | (including serine-arginine (SR) |
| | | linker at N-terminus and leucine- |
| | | phenylalanine (LF) linker at |
| | | C-terminus of cpGFP) |
| 302-303 | ITALIC | serine-arginine (SR) linker at N- |
| and | CAPITALS | terminus and leucine-phenylalanine |
| 545-546 | | (LF) linker at C-terminus of cpGFP |

nucleic acid sequence of circularly permuted
green fluorescent protein
SEQ ID NO: 29
AACGTCTATATCAAGGCCGACAAGCAGAAGAACGGCATCAAGGCGAAC
TTCAAGATCCGCCACAACATCGAGGACGGCGGCGTGCAGCTCGCCTAC
CACTACCAGCAGAACACCCCCATCGGCGACGGCCCCGTGCTGCTGCCC
GACAACCACTACCTGAGCGTGCAGTCCAAACTTTCGAAAGACCCCAAC
GAGAAGCGCGATCACATGGTCCTGCTGGAGTTCGTGACCGCCGCCGGG
ATCACTCTCGGCATGGACGAGCTGTACAAGGGCGGTACCGGAGGGAGC
ATGGTGAGCAAGGGCGAGGAGCTGTTCACCGGGGTGGTGCCCATCCTG
GTCGAGCTGGACGGCGACGTAAACGGCCACAAGTTCAGCGTGTCCGGC
GAGGGTGAGGGCGATGCCACCTACGGCAAGCTGACCCTGAAGTTCATC
TGCACCACCGGCAAGCTGCCCGTGCCCTGGCCCACCCTCGTGACCACC
CTGACCTACGGCGTGCAGTGCTTCAGCCGCTACCCCGACCACATGAAG
CAGCACGACTTCTTCAAGTCCGCCATGCCCGAAGGCTACATCCAGGAG
CGCACCATCTTCTTCAAGGACGACGGCAACTACAA(G/C)
Dopamine Receptor D1 (DRD1) containing cpFP
sensor replacing all or part of 3^{rd}
intracellular loop
SEQ ID NO: 30
MRTLNTSAMDGTGLVVERDFSVRILTACFLSLLILSTLLGNTLVCAAV
IRFRHLRSKVINFFVISLAVSDLLVAVLVMPWKAVAEIAGFWPFGSFC
NIWVAFDIMCSTASILNLCVISVDRYWAISSP [symbol] RYERKMTPKAAFILI
SVAWTLSVLISFIPVQLSWHKAKPTSPSDGNATSLAETIDNCDSSLSR
TYAISSSVISFYIPVAIMIVTYTRIYRIAQKQLQKIDLSSLInvyika
dkqkngikanfkirhniedggvqlayhyqqntpigdgpvllpdnhyls
vqsklskdpnekrdhmvllefvtaagitlgmdelykggtggsmvskge
elftgvvpilveldgdvnghkfsvsgegegdatygkltlkficttgkl
pvpwptlvttltygvqcfsrypdhmkqhdffksampegyiqertiffk
ddgnyktraevkfegdtlvnrielkgidfkedgnilghkleynNHDQL
KRETKVLKTLSVIMGVFVCCWLPFFILNCILPFCGSGETQPFCIDSNT
FDVFVWFGWANSSLNPIIYAFNADFRKAFSTLLGCYRLCPATNNAIET
VSINNNGAAMFSSHHEPRGSISKECNLVYLIPHAVGSSEDLKKEEAAG
IARPLEKLSPALSVILDYDTDVS[L/M]EKIQPITQNGQHPT*

Feature Annotations for SEQ ID NO: 30

| Residue | | |
|---|---|---|
| numbers | Formatting | Feature definition |
| 129 | Bold and | X is any amino acid or an amino acid |
| | italic | selected from the group consisting |
| | | of F, A, G, V, I, L, M, S, T |
| 224-234 | Bold and | Optional long linker at N-terminus |
| | underlined | of cpFP |
| 230-234 | Bold and | Optional short linker at N-terminus |
| | underlined | of cpFP |
| 235-475 | Lower case | cpGFP or any other cpFP described herein |
| 476-480 | Bold and | Linker at C-terminus of cpFP |
| | underlined | |

adrenoceptor beta 1 (ADRB1, ADRBIR, B1AR,
BETALAR) containing cpFP sensor replacing all or
part of 3rd intracellular loop
SEQ ID NO: 31
MGAGVLVLGASEPGNLSSAAPLPDGAATAARLLVPASPPASLLPPASE
SPEPLSQQWTAGMGLLMALIVLLIVAGNVLVIVAIAKTPRLQTLTNLF
IMSLASADLVMGLLVVPFGATIVVWGRWEYGSFFCELWTSVDVLCVTA
SIETLCVIALDRYLAITSP [symbol] RYQSLLTRARARGLVCTVWAISALVSFL
PILMHWWRAESDEARRCYNDPKCCDFVTNRAYAIASSVVSFYVPLCIM
AFVYLRVFREAQKQLQKIDLSSLInvyikadkqkngikanfkirhnie
dggvqlayhyqqntpigdgpvllpdnhylsvqsklskdpnekrdhmvl
lefvtaagitlgmdelykggtggsmvskgeelftgvvpilveldgdvn
ghkfsvsgegegdatygkltlkficttgklpvpwptlvttltygvqcf
srypdhmkqhdffksampegyiqertiffkddgnyktraevkfegdtl
vnrielkgidfkedgnilghkleynNHDQLREQKALKTLGIIMGVFTL
CWLPFFLANVVKAFHRELVPDRLFVFFNWLGYANSAFNPIIYCRSPDF
RKAFQGLLCCARRAARRRHATHGDRPRASGCLARPGPPPSPGAASDDD
DDDVVGATPPARLLEPWAGCNGGAAADSDSSLDEPCRPGFASESKV*

Feature Annotations for SEQ ID NO: 31

| Residue | | |
|---|---|---|
| numbers | Formatting | Feature definition |
| 164 | Bold and | X is any amino acid or an amino acid |
| | italic | selected from the group consisting |
| | | of F, A, G, V, I, L, M, S, T |
| 254-264 | Bold and | Optional long linker at N-terminus |
| | underlined | of cpFP |
| 260-264 | Bold and | Optional short linker at N-terminus of cpFP |
| | underlined | |
| 265-505 | Lower case | cpGFP or any other cpFP described herein |
| 506-510 | Bold and | Linker at C-terminus of cpFP |
| | underlined | |

adrenoceptor beta 2 (ADRB2, B2AR; ADRB2R;
BETA2AR) containing cpFP sensor replacing all or
part of 3rd intracellular loop
SEQ ID NO: 32
MGQPGNGSAFLLAPNRSHAPDHDVTQQRDEVWVVGMGIVMSLIVLAIV
FGNVLVITAIAKFERLQTVTNYFITSLACADLVMGLAVVPFGAAHILM
KMWTFGNFWCEFWTSIDVLCVTASIETLCVIAVDRYFAITSP [symbol] KYQSL
LTKNKARVIILMVWIVSGLTSFLPIQMHWYRATHQEAINCYANETCCD
FFTNQAYAIASSIVSFYVPLVIMVFVYSRVFQEAKRQLQKIDLSSLIn
vyikadkqkngikanfkirhniedggvqlayhyqqntpigdgpvllpd
nhylsvqsklskdpnekrdhmvllefvtaagitlgmdelykggtggsm
vskgeelftgvvpilveldgdvnghkfsvsgegegdatygkltlkfic
ttgklpvpwptlvttltygvqcfsrypdhmkqhdffksampegyiqer
tiffkddgnyktraevkfegdtlvnrielkgidfkedgnilghkleyn
NHDQLKEHKALKTLGIIMGTFTLCWLPFFIVNIVHVIQDNLIRKEVYI
LLNWIGYVNSGFNPLIYCRSPDFRIAFQELLCLRRSRYPNVRPNNGYI
YNAHSWQSENREQSKGSSGDSDHAEGNLAKEECLSADKTDSNGNCSKA
QMRVL*

Feature Annotations for SEQ ID NO: 32

| Residue | | |
|---|---|---|
| numbers | Formatting | Feature definition |
| 139 | Bold and | X is any amino acid or an amino acid |
| | italic | selected from the group consisting |
| | | of F, A, G, V, I, L, M, S, T |
| 229-239 | Bold and | Optional long linker at N-terminus |
| | underlined | of cpFP |
| 235-239 | Bold and | Optional short linker at N-terminus of cpFP |
| | underlined | |
| 240-480 | Lower case | cpGFP or any other cpFP described herein |
| 481-485 | Bold and | Linker at C-terminus of cpFP |
| | underlined | |

5-hydroxytryptamine receptor 2A (HTR2A, 5-HT2A)
containing cpFP sensor replacing all or part of
3rd intracellular loop
SEQ ID NO: 33
MDILCEENTSLSSTTNSLMQLNDDTRLYSNDFNSGEANTSDAFNWTVD
SENRTNLSCEGCLSPSCLSLLHLQEKNWSALLTAVVIILTIAGNILVI
MAVSLEKKLQNATNYFLMSLAIADMLLGFLVMPVSMLTILYGYRWPLP
SKLCAVWIYLDVLFSTASIMHLCAISLDRYVAIQNP [symbol] HHSRFNSRTKA
FLKIIAVWTISVGISMPIPVFGLQDDSKVFKEGSCLLADDNFVLIGSF
VSFFIPLTIMVITYFLTIKSLQKQLQKIDLSSLInvyikadkqkngik
anfkirhniedggvqlayhyqqntpigdgpvllpdnhylsvqsklskd
pnekrdhmvllefvtaagitlgmdelykggtggsmvskgeelftgvvp
ilveldgdvnghkfsvsgegegdatygkltlkficttgklpvpwptlv
ttltygvqcfsrypdhmkqhdffksampegyiqertiffkddgnyktr
aevkfegdtlvnrielkgidfkedgnilghkleynNHDQLNEQKACKV
LGIVFFLFVVMWCPFFITNIMAVICKESCNEDVIGALLNVFVWIGYLS
SAVNPLVYTLFNKTYRSAFSRYIQCQYKENKKPLQLILVNTIPALAYK
SSQLQMGQKKNSKQDAKTTDNDCSMVALGKQHSEEASKDNSDGVNEKV
SCV*

Feature Annotations for SEQ ID NO: 33

| Residue | | |
|---|---|---|
| numbers | Formatting | Feature definition |
| 181 | Bold and | X is any amino acid or an amino acid |
| | italic | selected from the group consisting |
| | | of F, A, G, V, I, L, M, S, T |
| 264-274 | Bold and | Optional long linker at N-terminus of cpFP |
| | underlined | |
| 270-274 | Bold and | Optional short linker at N-terminus of cpFP |
| | underlined | |
| 275-515 | Lower case | cpGFP or any other cpFP described herein |
| 516-520 | Bold and | Linker at C-terminus of cpFP |
| | underlined | |

adenosine A2a receptor (ADORA2A, A2aR)
3rd containing cpFP sensor replacing all or part
of intracellular loop
SEQ ID NO: 34
MPIMGSSVYITVELAIAVLAILGNVLVCWAVWLNSNLQNVTNYFVVSL
AAADIAVGVLAIPFAITISTGFCAACHGCLFIACFVLVLTQSSIFSLL
AIAIDRYIAIRIP [symbol] RYNGLVTGTRAKGIIAICWVLSFAIGLTPMLGWN
NCGQPKEGKNHSQGCGEGQVACLFEDVVPMNYMVYFNFFACVLVPLLL
MLGVYLRIFLAARRQLQKIDLSSLInvyikadkqkngikanfkirhni
edggvqlayhyqqntpigdgpvllpdnhylsvqsklskdpnekrdhmv
llefvtaagitlgmdelykggtggsmvskgeelftgvvpilveldgdv
nghkfsvsgegegdatygkltlkficttgklpvpwptlvttltygvqc
fsrypdhmkqhdffksampegyiqertiffkddgnyktraevkfegdt
lvnrielkgidfkedgnilghkleynNHDQLKEVHAAKSLAIIVGLFA
LCWLPLHIINCFTFFCPDCSHAPLWLMYLAIVLSHTNSVVNPFIYAYR
IREFRQTFRKIIRSHVLRQQEPFKAAGTSARVLAAHGSDGEQVSLRLN
GHPPGVWANGSAPHPERRPNGYALGLVSGGSAQESQGNTGLPDVELLS
HELKGVCPEPPGLDDPLAQDGAGVS*

Feature Annotations for SEQ ID NO: 34

| Residue | | |
|---|---|---|
| numbers | Formatting | Feature definition |
| 110 | Bold and | X is any amino acid or an amino acid |
| | italic | selected from the group consisting |
| | | of F, A, G, V, I, L, M, S, T |
| 207-217 | Bold and | Optional long linker at N-terminus of cpFP |
| | underlined | |
| 213-217 | Bold and | Optional short linker at N-terminus of cpFP |
| | underlined | |
| 218-458 | Lower case | cpGFP or any other cpFP described herein |
| 459-463 | Bold and | Linker at C-terminus of cpFP |
| | underlined | |

adrenoceptor alpha 2A (ADRA2A, ADRA2; ADRAR;
ADRA2R) containing cpFP sensor replacing all or
part of 3rd intracellular loop
SEQ ID NO: 35
MGSLQPDAGNASWNGTEAPGGGARATPYSLQVTLTLVCLAGLLMLLTV
FGNVLVIIAVFTSRALKAPQNLFLVSLASADILVATLVIPFSLANEVM
GYWYFGKAWCEIYLALDVLFCTSSIVHLCAISLDRYWSITQA [symbol] EYNLK
RTPRRIKAIIITVWVISAVISFPPLISIEKKGGGGGPQPAEPRCEIND
QKWYVISSCIGSFFAPCLIMILVYVRIYQIAKRQLQKIDLSSLInvyi
kadkqkngikanfkirhniedggvqlayhyqqntpigdgpvllpdnhy
lsvqsklskdpnekrdhmvllefvtaagitlgmdelykggtggsmvsk
geelftgvvpilveldgdvnghkfsvsgegegdatygkltlkficttg
klpvpwptlvttltygvqcfsrypdhmkqhdffksampegyiqertif
fkddgnyktraevkfegdtlvnrielkgidfkedgnilghkleynNHD
QLREKRFTFVLAVVIGVFVVCWFPFFFTYTLTAVGCSVPRTLFKFFFW
FGYCNSSLNPVIYTIFNHDFRRAFKKILCRGDRKRIV*

Feature Annotations for SEQ ID NO: 35

| Residue | | |
|---|---|---|
| numbers | Formatting | Feature definition |
| 139 | Bold and | X is any amino acid or an amino acid |
| | italic | selected from the group consisting |
| | | of F, A, G, V, I, L, M, S, T |
| 226-236 | Bold and | Optional long linker at N-terminus of cpFP |
| | underlined | |
| 232-236 | Bold and | Optional short linker at N-terminus |
| | underlined | of cpFP |
| 237-477 | Lower case | cpGFP or any other cpFP described herein |
| 476-482 | Bold and | Linker at C-terminus of cpFP |
| | underlined | |

opioid receptor kappa 1 (OPRK1, KOR1)
containing cpFP sensor replacing all or part of
3rd intracellular loop
SEQ ID NO: 36
MDSPIQIFRGEPGPTCAPSACLPPNSSAWFPGWAEPDSNGSAGSEDAQ
LEPAHISPAIPVIITAVYSVVFVVGLVGNSLVMFVIIRYTKMKTATNI
YIFNLALADALVTTTMPFQSTVYLMNSWPFGDVLCKIVISIDYYNMFT
SIFTLTMMSVDRYIAVCHP [symbol] KALDFRTPLKAKIINICIWLLSSSVGIS
AIVLGGTKVREDVDVIECSLQFPDDDYSWWDLFMKICVFIFAFVIPVL
IIIVCYTLMILRLKSQLQKIDLSSLInvyikadkqkngikanfkirhn
iedggvqlayhyqqntpigdgpvllpdnhylsvqsklskdpnekrdhm
vllefvtaagitlgmdelykggtggsmvskgeelftgvvpilveldgd
vnghkfsvsgegegdatygkltlkficttgklpvpwptlvttltygvq
cfsrypdhmkqhdffksampegyiqertiffkddgnyktraevkfegd
tlvnrielkgidfkedgnilghkleynNHDQLREKDRNLRRITRLVLV
VVAVFVVCWTPIHIFILVEALGSTSHSTAALAAYYFCIALGYTNAALN
PILYAFLDENFKRCFRDFCFPLKMRMERQATARVRNTVQDPAYLRDID
GMNKPV

Feature Annotations for SEQ ID NO: 36

| Residue | | |
|---|---|---|
| numbers | Formatting | Feature definition |
| 164 | Bold and | X is any amino acid or an amino acid |
| | italic | selected from the group consisting |
| | | of F, A, G, V, I, L, M, S, T |
| 256-266 | Bold and | Optional long linker at N-terminus |
| | underlined | of cpFP |
| 262-266 | Bold and | Optional short linker at N-terminus |
| | underlined | of cpFP |
| 267-507 | Lower case | cpGFP or any other cpFP described herein |
| 508-512 | Bold and | Linker at C-terminus of cpFP |
| | underlined | |

opioid receptor mu 1 (OPRM1, MOR1) containing
cpFP sensor replacing all or part of 3rd
intracellular loop
SEQ ID NO: 37
MDSSAAPTNASNCTDALAYSSCSPAPSPGSWVNLSHLDGNLSDPCGPN
RTDLGGRDSLCPPTGSPSMITAITIMALYSIVCVVGLFGNFLVMYVIV
RYTKMKTATNIYIFNLALADALATSTLPFQSVNYLMGTWPFGTILCKI
VISIDYYNMFTSIFTLCTMSVDRYIAVCHP [symbol] KALDFRTPRNAKIINVC
NWILSSAIGLPVMFMATTKYRQGSIDCTLTFSHPTWYWENLLKICVFI
FAFIMPVLIITVCYGLMILRLKSQLQKIDLSSLInvyikadkqkngik
anfkirhniedggvqlayhyqqntpigdgpvllpdnhylsvqsklskd
pnekrdhmvllefvtaagitlgmdelykggtggsmvskgeelftgvvp
ilveldgdvnghkfsvsgegegdatygkltlkficttgklpvpwptlv
ttltygvqcfsrypdhmkqhdffksampegyiqertiffkddgnyktr
aevkfegdtlvnrielkgidfkedgnilghkleynNHDQLKEKDRNLR
RITRMVLVVVAVFIVCWTPIHIYVIIKALVTIPETTFQTVSWHFCIAL
GYTNSCLNPVLYAFLDENFKRCFREFCIPTSSNIEQQNSTRIRQNTRD
HPSTANTVDRTNHQLENLEAETAPLP*

Feature Annotations for SEQ ID NO: 37

| Residue | | |
|---|---|---|
| numbers | Formatting | Feature definition |
| 175 | Bold and | X is any amino acid or an amino acid |
| | italic | selected from the group consisting |
| | | of F, A, G, V, I, L, M, S, T |
| 264-274 | Bold and | Optional long linker at N-terminus |
| | underlined | of cpFP |
| 270-274 | Bold and | Optional short linker at N-terminus |
| | underlined | of cpFP |
| 275-515 | Lower case | cpGFP or any other cpFP described herein |
| 516-520 | Bold and | Linker at C-terminus of cpFP |
| | underlined | |

opioid receptor delta 1 (OPRD1, DOR1)
containing cpFP sensor replacing all or part of
3rd intracellular loop
SEQ ID NO: 38
MEPAPSAGAELQPPLFANASDAYPSACPSAGANASGPPGARSASSLAL
AIAITALYSAVCAVGLLGNVLVMFGIVRYTKMKTATNIYIFNLALADA
LATSTLPFQSAKYLMETWPFGELLCKAVLSIDYYNMFTSIFTATMMSV
DRYIAVCHP [symbol] KALDFRTPAKAKLINICIWVLASGVGVPIMVMAVTRPR
DGAVVCMLQFPSPSWYWDTVTKICVFLFAFVVPILIITVCYGLMLLRL
RSQLQKIDLSSLInvyikadkqkngikanfkirhniedggvqlayhyq
qntpigdgpvllpdnhylsvqsklskdpnekrdhmvllefvtaagitl
gmdelykggtggsmvskgeelftgvvpilveldgdvnghkfsvsgege
gdatygkltlkficttgklpvpwptlvttltygvqcfsrypdhmkqhd
ffksampegyiqertiffkddgnyktraevkfegdtlvnrielkgidf
kedgnilghkleynNHDQLKEKDRSLRRITRMVLVVVGAFVVCWAPIH
IFVIVWTLVDIDRRDPLVVAALHLCIALGYANSSLNPVLYAFLDENFK
RCFRQLCRA*

Feature Annotations for SEQ ID NO: 38

| Residue | | |
|---|---|---|
| numbers | Formatting | Feature definition |
| 154 | Bold and | X is any amino acid or an amino acid |
| | italic | selected from the group consisting |
| | | of F, A, G, V, I, L, M, S, T |
| 243-253 | Bold and | Optional long linker at N-terminus of cpFP |
| | underlined | |
| 249-253 | Bold and | Optional short linker at N-terminus |
| | underlined | of cpFP |
| 254-494 | Lower case | cpGFP or any other cpFP described herein |
| 495-499 | Bold and | Linker at C-terminus of cpFP |
| | underlined | |

melatonin receptor 1B (MTNR1B, MT2R)
containing cpFP sensor replacing all or part
of 3rd intracellular loop
SEQ ID NO: 39
MSENGSFANCCEAGGWAVRPGWSGAGSARPSRTPRPPWVAPALSAVLI
VTTAVDVVGNLLVILSVLRNRKLRNAGNLFLVSLALADLVVAFYPYPL
ILVAIFYDGWALGEEHCKASAFVMGLSVIGSVFNITALAINRYCYICH
S [symbol] AYHRIYRRWHTPLHICLIWLLTVVALLPNFFVGSLEYDPRIYSCTF
IQTASTQYTAAVVVIHFLLPIAVVSFCYLRIWVLVLQARRQLQKIDLS
SLInvyikadkqkngikanfkirhniedggvqlayhyqqntpigdgpv
llpdnhylsvqsklskdpnekrdhmvllefvtaagitlgmdelykggt
ggsmvskgeelftgvvpilveldgdvnghkfsvsgegegdatygkltl
kficttgklpvpwptlvttltygvqcfsrypdhmkqhdffksampegy
iqertiffkddgnyktraevkfegdtlvnrielkgidfkedgnilghk
leynNHDQLKPSDLRSFLTMFVVFVIFAICWAPLNCIGLAVAINPQEM
APQIPEGLFVTSYLLAYFNSCLNAIVYGLLNQNFRREYKRILLALWNP
RHCIQDASKGSHAEGLQSPAPPIIGVQHQADAL*

Feature Annotations for SEQ ID NO: 39

| Residue | | |
|---|---|---|
| numbers | Formatting | Feature definition |
| 146 | Bold and | X is any amino acid or an amino acid |
| | italic | selected from the group consisting |
| | | of F, A, G, V, I, L, M, S, T |
| 233-243 | Bold and | Optional long linker at N-terminus of cpFP |
| | underlined | |
| 239-243 | Bold and | Optional short linker at N-terminus of cpFP |
| | underlined | |
| 244-484 | Lower case | cpGFP or any other cpFP described herein |
| 485-489 | Bold and | Linker at C-terminus of cpFP |
| | underlined | |

-Cannabinoid Receptor (type-1) (CNR1, CB1; CNR;
CB-R; CB1A; CB1R; CANN6; CB1K5) containing cpFP sensor
replacing all or part of 3rd intracellular loop
SEQ ID NO: 40
MKSILDGLADTTFRTITTDLLYVGSNDIQYEDIKGDMASKLGYFPQKFPLTSFRGSPFQEKM
TAGDNPQLVPADQVNITEFYNKSLSSFKENEENIQCGENFMDIECFMVLNPSQQLAIAVLSL
TLGTFTVLENLLVLCVILHSRSLRCRPSYHFIGSLAVADLLGSVIFVYSFIDFHVFHRKDSR
NVFLFKLGGVTASFTASVGSLFLTAIDRYISIHRP [symbol] AYKRIVTRPKAVVAFCLMWTIAIVIA
VLPLLGWNCEKLQSVCSDIFHIDETYLMFWIGVTSVLLLFIVYAYMYILWKAHSHAVRMIQ
RQLQKIDLSSLInvyikadkqkngikanfkirhniedggvqlayhyqqntpigdgpvllpdn
hylsvqsklskdpnekrdhmvllefvtaagitlgmdelykggtggsmvskgeelftgvvpil
veldgdvnghkfsvsgegegdatygkltlkficttgklpvpwptlvttltygvqcfsrypdh
mkqhdffksampegyiqertiffkddgnyktraevkfegdtlvnrielkgidfkedgnilgh
kleynNHDQLRMDIRLAKTLVLILVVLIICWGPLLAIMVYDVFGKMNKLIKTVFAFCSMLCL
LNSTVNPIIYALRSKDLRHAFRSMFPSCEGTAQPLDNSMGDSDCLHKHANNAASVHRAAESC
IKSTVKIAKV*

Feature Annotations for SEQ ID NO: 40

| Residue | | |
|---|---|---|
| numbers | Formatting | Feature definition |
| 222 | Bold and | X is any amino acid or an amino acid |
| | italic | selected from the group consisting |
| | | of F, A, G, V, I, L, M, S, T |
| 312-322 | Bold and | Optional long linker at N-terminus |
| | underlined | of cpFP |
| 318-322 | Bold and | Optional short linker at N-terminus of cpFP |
| | underlined | |
| 323-563 | Lower case | cpGFP or any other cpFP described herein |
| 564-568 | Bold and | Linker at C-terminus of cpFP |
| | underlined | |

-histamine receptor H1 (HRH1, H1R; H1-R; HH1R; hisH1)
containing cpFP sensor replacing all or part of 3rd
intracellular loop
SEQ ID NO: 41
MSLPNSSCLLEDKMCEGNKTTMASPQLMPLVVVLSTICLVTVGLNLLVLYAVRSERKLHTVG
NLYIVSLSVADLIVGAVVMPMNILYLLMSKWSLGRPLCLFWLSMDYVASTASIFSVFILCID
RYRSVQQP [symbol] RYLKYRTKTRASATILGAWFLSFLWVIPILGWNHFMQQTSVRREDKCETDFYD
VTWFKVMTAIINFYLPTLLMLWFYAKIYKAVRQLQKIDLSSLInvyikadkqkngikanfki
rhniedggvqlayhyqqntpigdgpvllpdnhylsvqsklskdpnekrdhmvllefvtaagi
tlgmdelykggtggsmvskgeelftgvvpilveldgdvnghkfsvsgegegdatygkltlkf
icttgklpvpwptlvttltygvqcfsrypdhmkqhdffksampegyigertiffkddgnykt
raevkfegdtlvnrielkgidfkedgnilghkleynNHDQLRERKAAKQLGFIMAAFILCWI
PYFIFFMVIAFCKNCCNEHLHMFTIWLGYINSTLNPLIYPLCNENFKKTFKRILHIRS*

Feature Annotations for SEQ ID NO: 41

| Residue | | |
|---|---|---|
| numbers | Formatting | Feature definition |
| 133 | Bold and | X is any amino acid or an amino acid |
| | italic | selected from the group consisting |
| | | of F, A, G, V, I, L, M, S, T |
| 219-229 | Bold and | Optional long linker at N-terminus |
| | underlined | of cpFP |
| 225-229 | Bold and | Optional short linker at N-terminus of cpFP |
| | underlined | |
| 230-470 | Lower case | cpGFP or any other cpFP described herein |
| 471-475 | Bold and | Linker at C-terminus of cpFP |
| | underlined | |

-neuropeptide Y receptor Y1 (NPYIR, NPYR; NPY1-R)
containing cpFP sensor replacing all or part of 3rd
intracellular loop
SEQ ID NO: 42
MNSTLFSQVENHSVHSNFSEKNAQLLAFENDDCHLPLAMIFTLALAYGAVIILGVSGNLALI
IIILKQKEMRNVTNILIVNLSFSDLLVAIMCLPFTFVYTLMDHWVFGEAMCKLNPFVQCVSI
TVSIFSLVLIAVERHQLIINPRGWRPNNRHAYVGIAVIWVLAVASSLPFLIYQVMTDEPFQN
VTLDAYKDKYVCFDQFPSDSHRLSYTTLLLVLQYFGPLCFIFICYFKIYIRLKRRQLQKIDL
SSLInvyikadkqkngikanfkirhniedggvqlayhyqqntpigdgpvllpdnhylsvqsk
lskdpnekrdhmvllefvtaagitlgmdelykggtggsmvskgeelftgvvpilveldgdvn
ghkfsvsgegegdatygkltlkficttgklpvpwptlvttltygvqcfsrypdhmkqhdffk
sampegyigertiffkddgnyktraevkfegdtlvnrielkgidfkedgnilghkleynNHD
QLSETKRINIMLLSIVVAFAVCWLPLTIENTVEDWNHQIIATCNHNLLFLLCHLTAMISTCV
NPIFYGELNKNFQRDLQFFFNFCDERSRDDDYETIAMSTMHTDVSKTSLKQASPVAFKKINN
NDDNEKI

Feature Annotations for SEQ ID NO: 42

| Residue | | |
|---|---|---|
| numbers | Formatting | Feature definition |
| 242-252 | Bold and | Optional long linker at N- |
| | underlined | terminus of cpFP |
| 248-252 | Bold and | Optional short linker at N- |
| | underlined | terminus of cpFP |
| 253-493 | Lower case | cpGFP or any other cpFP described herein |
| 494-498 | Bold and | Linker at C-terminus of cpFP |
| | underlined | |

-cholinergic receptor muscarinic 2 (CHRM2, HM2)
containing cpFP sensor replacing all or part of 3rd
intracellular loop
SEQ ID NO: 43
MNNSTNSSNNSLALTSPYKTFEVVFIVLVAGSLSLVTIIGNILVMVSIKVNRHLQTVNNYFL
FSLACADLIIGVFSMNLYTLYTVIGYWPLGPVVCDLWLALDYVVSNASVMNLLIISEDRYFC
VTKP [symbol] TYPVKRTTKMAGMMIAAAWVLSFILWAPAILFWQFIVGVRTVEDGECYIQFFSNAAV
TFGTAIAAFYLPVIIMTVLYWHISRASKSQLQKIDLSSLInvyikadkqkngikanfkirhn
iedggvqlayhyqqntpigdgpvllpdnhylsvqsklskdpnekrdhmvllefvtaagitlg
mdelykggtggsmvskgeelftgvvpilveldgdvnghkfsvsgegegdatygkltlkfict
tgklpvpwptlvttltygvqcfsrypdhmkqhdffksampegyiqertiffkddgnyktrae
vkfegdtlvnrielkgidfkedgnilghkleynNHDQLREKKVTRTILAILLAFIITWAPYN
VMVLINTFCAPCIPNTVWTIGYWLCYINSTINPACYALCNATFKKTFKHLLMCHYKNIGATR

Feature Annotations for SEQ ID NO: 43

| Residue | | |
|---|---|---|
| numbers | Formatting | Feature definition |
| 129 | Bold and | X is any amino acid or an amino acid |
| | italic | selected from the group consisting |
| | | of F, A, G, V, I, L, M, S, T |
| 216-226 | Bold and | Optional long linker at N-terminus |
| | underlined | of cpFP |
| 222-226 | Bold and | Optional short linker at N-terminus of cpFP |
| | underlined | |
| 227-467 | Lower case | cpGFP or any other cpFP described herein |
| 468-472 | Bold and | Linker at C-terminus of cpFP |
| | underlined | |

-hypocretin (orexin) receptor 1 (HCRTR1, OX1R)
containing cpFP sensor replacing all or part of 3rd
intracellular loop
SEQ ID NO: 44
MEPSATPGAQMGVPPGSREPSPVPPDYEDEFLRYLWRDYLYPKQYEWVLIAAYVAVFVVALV
GNTLVCLAVWRNHHMRTVTNYFIVNLSLADVLVTAICLPASLLVDITESWLFGHALCKVIPY
LQAVSVSVAVLTLSFIALDRWYAICHP [symbol] LFKSTARRARGSILGIWAVSLAIMVPQAAVMECS
SVLPELANRTRLFSVCDERWADDLYPKIYHSCFFIVTYLAPLGLMAMAYFQIFRKLWGRQLQ
KIDLSSLInvyikadkqkngikanfkirhniedggvqlayhyqqntpigdgpvllpdnhyls
vqsklskdpnekrdhmvllefvtaagitlgmdelykggtggsmvskgeelftgvvpilveld
gdvnghkfsvsgegegdatygkltlkficttgklpvpwptlvttltygvqcfsrypdhmkqh
dffksampegyigertiffkddgnyktraevkfegdtlvnrielkgidfkedgnilghkley
nNHDQLRARRKTAKMLMVVLLVFALCYLPISVLNVLKRVFGMFRQASDREAVYACFTESHWL
VYANSAANPIIYNFLSGKFREQFKAAFSCCLPGLGPCGSLKAPSPRSSASHKSLSLQSRCSI
SKISEHVVLTSVTTVLP

Feature Annotations for SEQ ID NO: 44

| Residue | | |
|---|---|---|
| numbers | Formatting | Feature definition |
| 152 | Bold and | X is any amino acid or an amino acid |
| | italic | selected from the group consisting |
| | | of F, A, G, V, I, L, M, S, T |
| 246-256 | Bold and | Optional long linker at N-terminus |
| | underlined | of cpFP |
| 252-256 | Bold and | Optional short linker at N-terminus |
| | underlined | of cpFP |
| 257-497 | Lower case | cpGFP or any other cpFP described herein |
| 498-502 | Bold and | Linker at C-terminus of cpFP |
| | underlined | |

-nucleic acid sequence of Dopamine Receptor D2
(DRD2) containing cpFP sensor replacing all or part of 3rd
intracellular loop
SEQ ID NO: 45
ATGGACCCCCTTAACCTCTCATGGTACGACGATGATCTTGAGAGGCAGAACTGGTCCCGACC
ATTCAATGGGTCTGATGGTAAGGCTGACCGGCCTCATTACAATTATTATGCGACCCTGCTTA
CTCTTCTTATCGCTGTGATCGTATTCGGCAACGTCTTGGTTTGCATGGCAGTCTCTAGGGAA
AAAGCGCTCCAGACGACAACTAATTACTTGATTGTGAGTCTGGCTGTAGCTGACTTGCTTGT
GGCGACCCTGGTGATGCCATGGGTCGTATACTTGGAAGTCGTTGGCGAGTGGAAGTTTTCTA
GGATTCATTGCGACATATTTGTAACTCTGGACGTAATGATGTGTACTGCTTCCATTTTGAAC
CTCTGCGCTATATCCATTGACAGGTACACGGCGGTTGCTATGCCGATGCTTTATAATACCCG
GTATTCAAGCAAAAGGCGAGTAACTGTGATGATAAGCATTGTATGGGTGCTCAGTTTCACAA
TTAGCTGCCCTCTGCTCTTCGGCCTTAACAACGCGGATCAAAATGAATGCATCATCGCAAAC
CCGGCTTTTGTGGTTTATAGCAGCATTGTTAGCTTCTATGTGCCATTCATAGTTACGCTCCT
TGTTTATATAAAAATTTATATCGTGCTTAGGCGCCGCCGAAAACGAGTTAACACCAAGCGGA
GCAGCCTGAGCTCActcattAATGTATATATCAAAGCTGATAAGCAAAAAAACGGTATCAAG
GCTAATTTTAAGATCAGACATAATATAGAGGATGGAGGCGTTCAACTGGCCTACCACTACCA
GCAAAACACGCCGATCGGGGATGGGCCAGTACTTCTGCCAGATAACCATTATCTCTCAGTTC
AAAGCAAACTCTCTAAGGACCCTAATGAGAAACGAGATCATATGGTTCTGCTCGAATTCGTT
ACAGCCGCCGGTATCACACTTGGGATGGACGAGTTGTATAAGGGTGGAACAGGAGGGTCAAT
GGTAAGCAAAGGCGAGGAGCTGTTTACGGGGGTCGTCCCGATACTTGTTGAACTCGACGGCG
ATGTCAACGGGCACAAATTCTCAGTGAGTGGCGAGGGGGAAGGAGACGCCACTTATGGAAAA
CTGACATTGAAATTCATATGTACGACTGGGAAGTTGCCTGTGCCTTGGCCTACGCTCGTTAC
TACACTTACTTACGGGGTACAGTGTTTCAGTAGGTATCCAGATCACATGAAACAGCACGATT
TTTTCAAGAGTGCAATGCCGGAAGGATATATACAAGAAAGAACTATTTTCTTTAAAGATGAC
GGCAACTATAAAACGCGAGCAGAGGTGAAGTTTGAGGGCGATACCTTGGTTAATAGGATCGA
ACTCAAAGGCATAGACTTCAAAGAAGACGGAAACATTCTGGGTCACAAACTGGAATACAACa
atcatGACCAACTGCAGAAGGAAAAGAAGGCCACGCAAATGTTGGCAATCGTGCTCGGCGTG
TTCATAATCTGCTGGCTTCCATTTTTTATAACGCATATATTGAACATACACTGTGATTGCAA
TATTCCACCAGTCCTGTATAGTGCGTTTACGTGGTTGGGTTATGTGAATTCTGCGGTTAACC
CGATCATTTACACCACGTTCAACATAGAATTCCGAAAGGCATTCCTCAAAATATTGCATTGT
TAG
-amino acid sequence of Dopamine Receptor D2
(DRD2) containing cpFP sensor replacing all or part of 3rd
intracellular loop
SEQ ID NO: 46
MDPLNLSWYDDDLERQNWSRPENGSDGKADRPHYNYYATLLTLLIAVIVFGNVLVCMAVSRE
KALQTTTNYLIVSLAVADLLVATLVMPWVVYLEVVGEWKESRIHCDIFVTLDVMMCTASILN
LCAISIDRYTAVAMP [symbol] LYNTRYSSKRRVTVMISIVWVLSFTISCPLLFGLNNADQNECIIAN
PAFVVYSSIVSFYVPFIVTLLVYIKIYIVLRRRRKLSSLInvyikadkqkngikanfkirhn
iedggvqlayhyqqntpigdgpvllpdnhylsvqsklskdpnekrdhmvllefvtaagitlg
mdelykggtggsmvskgeelftgvvpilveldgdvnghkfsvsgegegdatygkltlkfict
tgklpvpwptlvttltygvqcfsrypdhmkqhdffksampegyigertiffkddgnyktrae
vkfegdtlvnrielkgidfkedgnilghkleynNHDQLQKEKKATQMLAIVLGVFIICWLPF
FITHILNIHCDCNIPPVLYSAFTWLGYVNSAVNPIIYTTFNIEFRKAFLKILHC*

Feature Annotations for SEQ ID NO: 46

| Residue | | |
|---|---|---|
| numbers | Formatting | Feature definition |
| 140 | Bold and | X is any amino acid or an amino acid |
| | italic | selected from the group consisting |
| | | of F, A, G, V, I, L, M, S, T |
| 222-226 | Bold and | linker at N-terminus of cpFP |
| | underlined | |
| 235-475 | Lower case | cpGFP or any other cpFP described herein |
| 476-480 | Bold and | Linker at C-terminus of cpFP |
| | underlined | |

-nucleic acid sequence of Dopamine Receptor D4
(DRD4) containing cpFP sensor replacing all or part of 3rd
intracellular loop
SEQ ID NO: 47
ATGGGGAACAGATCCACTGCAGATGCAGACGGTCTTCTCGCAGGCCGGGGACCTGCTGCCGG
AGCGAGCGCTGGGGCTTCCGCAGGTCTTGCTGGGCAGGGGGCCGCGGCCTTGGTTGGAGGCG
TTTTGCTTATAGGGGCCGTTCTTGCTGGCAATAGTTTGGTATGTGTTTCAGTTGCGACAGAG
CGCGCACTTCAGACGCCGACTAACTCCTTTATAGTGAGTTTGGCTGCTGCAGATCTCTTGTT
GGCATTGTTGGTACTCCCACTGTTCGTTTATTCAGAAGTACAGGGTGGCGCATGGCTCCTGT
CACCCAGGTTGTGTGATGCCTTGATGGCCATGGATGTTATGCTGTGTACCGCTTCTATCTTT
AACCTTTGTGCTATCAGTGTTGACAGATTCGTCGCGGTCGCGGTCCCTCTGAGGTATAACCG
GCAAGGAGGCAGCAGGAGGCAACTGCTGCTGATCGGCGCAACTTGGCTCCTCTCCGCAGCAG
TGGCCGCGCCTGTTCTGTGTGGTCTCAACGACGTTCGCGGCAGAGACCCGGCTGTATGTCGC
CTCGAGGATAGAGATTATGTCGTATACTCAAGTGTGTGTTCCTTTTTTCTTCCTTGCCCACT
GATGCTTCTGTTGTATTGGGCTACCTTTAGAGGACTGCAACGCTGGGAAGTCCTGAGCTCAC
tcattAATGTATATATCAAAGCTGATAAGCAAAAAAACGGTATCAAGGCTAATTTTAAGATC
AGACATAATATAGAGGATGGAGGCGTTCAACTGGCCTACCACTACCAGCAAAACACGCCGAT
CGGGGATGGGCCAGTACTTCTGCCAGATAACCATTATCTCTCAGTTCAAAGCAAACTCTCTA
AGGACCCTAATGAGAAACGAGATCATATGGTTCTGCTCGAATTCGTTACAGCCGCCGGTATC
ACACTTGGGATGGACGAGTTGTATAAGGGTGGAACAGGAGGGTCAATGGTAAGCAAAGGCGA
GGAGCTGTTTACGGGGGTCGTCCCGATACTTGTTGAACTCGACGGCGATGTCAACGGGCACA
AATTCTCAGTGAGTGGCGAGGGGGAAGGAGACGCCACTTATGGAAAACTGACATTGAAATTC
ATATGTACGACTGGGAAGTTGCCTGTGCCTTGGCCTACGCTCGTTACTACACTTACTTACGG
GGTACAGTGTTTCAGTAGGTATCCAGATCACATGAAACAGCACGATTTTTTCAAGAGTGCAA
TGCCGGAAGGATATATACAAGAAAGAACTATTTTCTTTAAAGATGACGGCAACTATAAAACG
CGAGCAGAGGTGAAGTTTGAGGGCGATACCTTGGTTAATAGGATCGAACTCAAAGGCATAGA
CTTCAAAGAAGACGGAAACATTCTGGGTCACAAACTGGAATACAACaatcatGACCAACTGG
GCCGCGAACGGAAAGCCATGCGAGTTTTGCCGGTGGTAGTAGGGGCATTCCTTCTTTGTTGG
ACCCCTTTTTTTGTGGTGCATATAACGCAGGCTCTGTGCCCGGCCTGTTCTGTCCCACCCCG
CCTCGTGTCAGCTGTCACTTGGTTGGGTTACGTAAACTCAGCCCTCAATCCAGTTATCTATA
CGGTTTTCAATGCCGAGTTCAGGAATGTTTTTAGGAAGGCCCTTAGAGCCTGTTGTTAG
-amino acid sequence of Dopamine Receptor D4
(DRD4) containing cpFP sensor replacing all or part of 3rd
intracellular loop
SEQ ID NO: 48
MGNRSTADADGLLAGRGPAAGASAGASAGLAGQGAAALVGGVLLIGAVLAGNSLVCVSVATE
RALQTPTNSFIVSLAAADLLLALLVLPLEVYSEVQGGAWLLSPRLCDALMAMDVMLCTASIF
NLCAISVDRFVAVAVP [symbol] RYNRQGGSRRQLLLIGATWLLSAAVAAPVLCGLNDVRGRDPAVCR
LEDRDYVVYSSVCSFFLPCPLMLLLYWATFRGLQRWEVLSSLInvyikadkqkngikanfki
rhniedggvqlayhyqqntpigdgpvllpdnhylsvqsklskdpnekrdhmvllefvtaagi
tlgmdelykggtggsmvskgeelftgvvpilveldgdvnghkfsvsgegegdatygkltlkf
icttgklpvpwptlvttltygvqcfsrypdhmkqhdffksampegyigertiffkddgnykt
raevkfegdtlvnrielkgidfkedgnilghkleynNHDQLGRERKAMRVLPVVVGAFLLCW
TPFFVVHITQALCPACSVPPRLVSAVTWLGYVNSALNPVIYTVENAEFRNVERKALRACC*

Feature Annotations for SEQ ID NO: 48

| Residue | | |
|---|---|---|
| numbers | Formatting | Feature definition |
| 141 | Bold and | X is any amino acid or an amino acid |
| | italic | selected from the group consisting |
| | | of F, A, G, V, I, L, M, S, T |
| 225-229 | Bold and | linker at N-terminus of cpFP |
| | underlined | |
| 230-470 | Lower case | cpGFP or any other cpFP described herein |
| 471-475 | Bold and | Linker at C-terminus of cpFP |
| | underlined | |

SEQ ID NO: 49
LSSGY-cpGFP-MHDQL
SEQ ID NO: 50
LSSLI-cpGFP-NHDQL
SEQ ID NO: 51
LSSX1X2-cpGFP-X3X4DQL

What is claimed is:

1. A method of detecting a ligand-induced hallucinogenic conformational change of a G Protein-Coupled Receptor (GPCR), the method comprising:
contacting the ligand with a fluorescent biosensor under conditions for the ligand to bind to the fluorescent biosensor, wherein the fluorescent biosensor comprises the GPCR, and a circularly permuted green fluorescent protein (cpGFP) integrated into the third intracellular loop (IL3) of the GPCR; and
measuring the change in fluorescence of the biosensor, thereby detecting the conformational change.

2. The method of claim 1, wherein the GPCR is a 5-HT receptor.

3. The method of claim 1 or 2, wherein the GPCR is a 5-HT2A receptor.

4. The method of claim 3, wherein the cpGFP is inserted between Lys²⁶³ and Ser³¹⁶ of the 5-HT2A receptor.

5. The method of any one of claims 1 to 4, wherein the 5-HT2A comprises the polypeptide LSSGY-cpGFP-MHDQL (SEQ ID NO:49).

6. The method of any one of claims 1 to 5, wherein the polypeptide LSSGY-cpGFP-MHDQL (SEQ ID NO:49) is inserted between Lys²⁶³ and Ser³¹⁶ of the 5-HT2A receptor.

7. The method of any one of claims 1 to 6, wherein the transmembrane helix 5 (TM5) comprises the point mutation E264Q.

8. The method of any one of claims 1 to 7, wherein the transmembrane helix 6 (TM6) comprises the deletion of Ser³¹⁶.

9. The method of any one of claims 1 to 8, wherein the intracellular loop 2 (ICL2) comprises the point mutation Ile^{181A}.

10. The method of any one of claims 1 to 9, wherein the cpGFP comprises GCaMP6.

11. The method of any one of claims 1 to 10, wherein the fluorescent biosensor further comprises an ER export peptide on the C-terminus.

12. The method of claim 11, wherein the ER export peptide is FCYENEV.

13. A method of detecting a hallucinogenic compound, the method comprising:
contacting a compound with a fluorescent biosensor under conditions for the compound to bind to the fluorescent biosensor, wherein the fluorescent biosensor comprises a G Protein-Coupled Receptor (GPCR), and a circularly permuted green fluorescent protein (cpGFP) integrated into the third intracellular loop (IL3) of the GPCR; and
measuring the change in fluorescence of the biosensor, wherein an increase in fluorescence indicates the presence of the hallucinogenic compound, thereby detecting the hallucinogenic compound.

14. The method of claim 13, wherein the GPCR is a 5-HT receptor.

15. The method of claim 13 or 14, wherein the GPCR is a 5-HT2A receptor.

16. The method of claim 15, wherein the cpGFP is inserted between Lys²⁶³ and Ser³¹⁶ of the 5-HT2A receptor.

17. The method of claim 15 or 16, wherein the 5-HT2A comprises the polypeptide LSSGY-cpGFP-MHDQL (SEQ ID NO:49).

18. The method of any one of claims 15 to 17, wherein the polypeptide LSSGY-cpGFP-MHDQL (SEQ ID NO:49) is inserted between Lys²⁶³ and Ser³¹⁶ of the 5-HT2A receptor.

19. The method of any one of claims 15 to 18, wherein the transmembrane helix 5 (TM5) comprises the point mutation E264Q.

20. The method of any one of claims 15 to 19, wherein the transmembrane helix 6 (TM6) comprises the deletion of Ser³¹⁶.

21. The method of any one of claims 15 to 20, wherein the intracellular loop 2 (ICL2) comprises the point mutation Ile^{181A}.

22. The method of any one of claims 13 to 16, wherein the cpGFP comprises GCaMP6.

23. The method of any one of claims 13 to 22, wherein the fluorescent biosensor further comprises an ER export peptide on the C-terminus.

24. The method of claim 23, wherein the ER export peptide is FCYENEV.

25. A method of detecting a non-hallucinogenic antidepressant compound, the method comprising:
contacting a compound with a fluorescent biosensor under conditions for the compound to bind to the fluorescent biosensor, wherein the fluorescent biosensor comprises a G Protein-Coupled Receptor (GPCR), and a circularly permuted green fluorescent protein (cpGFP) integrated into the third intracellular loop (IL3) of the GPCR; and
measuring the change in fluorescence of the biosensor, wherein a decrease in fluorescence indicates the presence of the non-hallucinogenic antidepressant compound, thereby detecting the non-hallucinogenic antidepressant compound.

26. The method of claim 25, wherein the GPCR is a 5-HT receptor.

27. The method of claim 25 or 26, wherein the GPCR is a 5-HT2A receptor.

28. The method of claim 27, wherein the cpGFP is inserted between Lys²⁶³ and Ser³¹⁶ of the 5-HT2A receptor.

29. The method of claim 27 or 28, wherein the 5-HT2A comprises the polypeptide LSSGY-cpGFP-MHDQL (SEQ ID NO:49).

30. The method of any one of claims 27 to 29, wherein the polypeptide LSSGY-cpGFP-MHDQL (SEQ ID NO:49) is inserted between Lys²⁶³ and Ser³¹⁶ of the 5-HT2A receptor.

31. The method of any one of claims 27 to 30, wherein the transmembrane helix 5 (TM5) comprises the point mutation E264Q.

32. The method of any one of claims 27 to 31, wherein the transmembrane helix 6 (TM6) comprises the deletion of Ser³¹⁶.

33. The method of any one of claims 27 to 32, wherein the intracellular loop 2 (ICL2) comprises the point mutation Ile¹⁸¹.

34. The method of any one of claims 25 to 33, wherein the cpGFP comprises GCaMP6.

35. The method of any one of claims 25 to 34, wherein the fluorescent biosensor further comprises an ER export peptide on the C-terminus.

36. The method of claim 35, wherein the ER export peptide is FCYENEV.

37. A method of identifying a hallucinogenic compound from a non-hallucinogenic compound, the method comprising:
contacting a compound with a fluorescent biosensor under agonist conditions and measuring a first fluorescence signal of the compound, wherein an increase in the first fluoresence signal compared to a first control indicates the compound is hallucinogenic;
contacting the compound with the fluorescent biosensor under antagonist conditions and measuring a second fluoresence signal of the compound, wherein a decreased second fluoresence signal compared to a second control indicates the compound is non-hallucinogenic; and
combining the first fluoresence signal and the second fluorescence signal to calculate a ligand score where a positive ligand score identifies the compound as a hallucinogenic compound and a negative ligand score identifies the compound as a non-hallucinogenic compound.

38. A fluorescent biosensor comprising:
a 5-HT2A receptor; and
a circularly permuted green fluorescent protein (cpGFP) integrated in the third intracellular loop (IL3) of the 5-HT2A receptor.

39. The fluorescent biosensor of claim 38, wherein the cpGFP is inserted between Lys²⁶³ and Ser³¹⁶ of the 5-HT2A receptor.

40. The fluorescent biosensor of claim 38 or 39, wherein the 5-HT2A comprises the polypeptide LSSGY-cpGFP-MHDQL (SEQ ID NO:49).

41. The fluorescent biosensor of any one of claims 38 to 40, wherein the polypeptide LSSGY-cpGFP-MHDQL (SEQ ID NO:49) is inserted between Lys²⁶³ and Ser³¹⁶ of the 5-HT2A receptor.

42. The fluorescent biosensor of any one of claims 38 to 41, wherein the 5-HT2A receptor comprises a transmembrane helix 5 (TM5) comprising a point mutation E264Q.

43. The fluorescent biosensor of any one of claims 38 to 42, wherein the 5-HT2A receptor comprises transmembrane helix 6 (TM6) comprising a deletion of Ser³¹⁶.

44. The fluorescent biosensor of any one of claims 38 to 43, wherein the 5-HT2A receptor comprises intracellular loop 2 (ICL2) comprising a point mutation Ile^{181A}.

45. The fluorescent biosensor of any one of claims 38 to 44, comprising:
the 5-HT2A receptor;
the polypeptide LSSGY-cpGFP-MHDQL (SEQ ID NO:49) inserted between Lys²⁶³ and Ser³¹⁶ of the 5-HT2A receptor;
the transmembrane helix 5 (TM5) of the 5-HT2A receptor comprises the point mutation E264Q;
the transmembrane helix 6 (TM6) of the 5-HT2A receptor comprises the deletion of Ser³¹⁶; and
the intracellular loop 2 (ICL2) of the 5-HT2A receptor comprises the point mutation Ile^{181A}.

46. The fluorescent biosensor of any one of claims 38 to 45, wherein the fluorescent biosensor comprises the sequence of SEQ ID NO:52.

47. The fluorescent biosensor of any one of claims 38 to 46, wherein the fluorescent biosensor further comprises an ER export peptide on the C-terminus.

48. The fluorescent biosensor of claim 47, wherein the ER export peptide is FCYENEV.

49. The fluorescent biosensor of any one of claims 38 to 48, comprising:
the 5-HT2A receptor;
the polypeptide LSSGY-cpGFP-MHDQL (SEQ ID NO:49) inserted between Lys²⁶³ and Ser³¹⁶ of the 5-HT2A receptor;
the transmembrane helix 5 (TM5) of the 5-HT2A receptor comprises the point mutation E264Q;
the transmembrane helix 6 (TM6) of the 5-HT2A receptor comprises the deletion of Ser³¹⁶;
the intracellular loop 2 (ICL2) of the 5-HT2A receptor comprises the point mutation Ile^{181A}; and
an ER export peptide on the C-terminus of the fluorescent biosensor, wherein the ER export peptide is FCYENEV.

50. The fluorescent biosensor of any one of claims 38 to 49, wherein the fluorescent biosensor comprises the sequence of SEQ ID NO:53.

51. A method of measuring the hallucinogenic potential of a compound, comprising contacting the compound with a fluorescent biosensor of any one of claims 38 to 50, and measuring the agonist effect of the compound on the fluorescent biosensor.

52. A method of measuring the antipsychotic potential of a compound, comprising contacting the compound with a fluorescent biosensor of any one of claims 38 to 50, and measuring the agonist or antagonist effect of the compound on the fluorescent biosensor.

53. A kit comprising a fluorescent biosensor of any one of claims 38 to 50.

54. A cell comprising a fluorescent biosensor of any one of claims 38 to 50.


SEQUENCE LISTING (53 sequences)

SEQ ID NO: 1 - protein, 241 aa, Artificial sequence
  Synthetic polypeptide
MSRVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 2 - protein, 239 aa, Artificial sequence
  Synthetic polypeptide
MVSKGEELFTGVVPILVELDGDVNGHKFSVSGEGEGDATYGKLTLKFICTTGKLPVPWPTLVTTLTWGVQCFSRYPDHMKQHDFFKSAMPEGYVQERTIFFKDDGNYKTRAEVKFEGDTLVNRIELKGIDFKEDGNILGHKLEYNYISHNVYITADKQKNGIKANFKIRHNIEDGSVQLADHYQQNTPIGDGPVLLPDNHYLSTQSALSKDPNEKRDHMVLLEFVTAAGITLGMDELYK

SEQ ID NO: 3 - protein, 239 aa, Artificial sequence
  Synthetic polypeptide
MVSKGEELFTGVVPILVELDGDVNGHKFSVSGEGEGDATYGKLTLKFICTTGKLPVPWPTLVTTFGYGLQCFARYPDHMKLHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSYQSALSKDPNEKRDHMVLLEFVTAAGITLGMDELYK

SEQ ID NO: 4 - protein, 239 aa, Artificial sequence
  Synthetic polypeptide
MVSKGEELFTGVVPILVELDGDVNGHKFSVSGEGEGDATYGKLTLKFICTTGKLPVPWPTLVTTFSYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKANFKIRHNIEDGSVQLADHYQQNTPIGDGPVLLPDNHYLSHQSALSKDPNEKRDHMVLLEFVTAAGITLGMDELYK

SEQ ID NO: 5 - protein, 238 aa, Artificial sequence
  Synthetic polypeptide
MSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 6 - protein, 228 aa, Artificial sequence
  Synthetic polypeptide
MVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHST

SEQ ID NO: 7 - protein, 236 aa, Artificial sequence
  Synthetic polypeptide
MVSKGEEDNMAIIKEFMRFKVHMEGSVNGHVFEIEGEGEGRPYEGTQTAKLKVTKGGPLPFTWDILSPQFMYGSNAYVKHPADIPDYFKLSFPEGFKWERVMKFEDGGVVTVTQDSSLQDGEFIYKVKLRGTNFPSDGPVMQKKTMGWEALSERMYPEDGALKGEVKPRVKLKDGGHYDAEVKTTYKAKKPVQLPGAYNVNRKLDITSHNEDYTIVEQYERAEGRHSTGGMDELYK

SEQ ID NO: 8 - protein, 236 aa, Artificial sequence
  Synthetic polypeptide
MVSKGEENNMAIIKEFMRFKVHMEGSVNGHEFEIEGEGEGRPYEAFQTAKLKVTKGGPLPFAWDILSPQFMYGSKVYIKHPADIPDYFKLSFPEGFRWERVMNFEDGGIIHVNQDSSLQDGVFIYKVKLRGTNFPSDGPVMQKKTMGWEASEERMYPEDGALKSEIKKRLKLKDGGHYAAEVKTTYKAKKPVQLPGAYIVDIKLDIVSHNEDYTIVEQYERAEGRHSTGGMDELYK

SEQ ID NO: 9 - protein, 237 aa, Artificial sequence
  Synthetic polypeptide
MVSKGEELIKENMRMKVVMEGSVNGHQFKCTGEGEGNPYMGTQTMRIKVIEGGPLPFAFDILATSFMYGSRTFIKYPKGIPDFFKQSFPEGFTWERVTRYEDGGVVTVMQDTSLEDGCLVYHVQVRGVNFPSNGPVMQKKTKGWEPNTEMMYPADGGLRGYTHMALKVDGGGHLSCSFVTTYRSKKTVGNIKMPGIHAVDHRLERLEESDNEMFVVQREHAVAKFAGLGGGMDELYK

SEQ ID NO: 10 - protein, 232 aa, Artificial sequence
  Synthetic polypeptide
MVSELIKENMHMKLYMEGTVNNHHFKCTSEGEGKPYEGTQTMRIKAVEGGPLPFAFDILATSFMYGSKTFINHTQGIPDFFKQSFPEGFTWERVTTYEDGGVLTATQDTSLQDGCLIYNVKIRGVNFPSNGPVMQKKTLGWEASTETLYPADGGLEGRADMALKLVGGGHLICNLKTTYRSKKPAKNLKMPGVYYVDRRLERIKEADKETYVEQHEVAVARYCDLPSKLGHR

SEQ ID NO: 11 - protein, 226 aa, Artificial sequence
  Synthetic polypeptide
MSAIKPDMKIKLRMEGNVNGHHFVIDGDGTGKPFEGKQSMDLEVKEGGPLPFAFDILTTAFHYGNRVFAKYPDNIQDYFKQSFPKGYSWERSLTFEDGGICIARNDITMEGDTFYNKVRFYGTNFPANGPVMQKKTLKWEPSTEKMYVRDGVLTGDIHMALLLEGNAHYRCDFRTTYKAKEKGVKLPGYHFVDHCIEILSHDKDYNKVKLYEHAVAHSGLPDNARR

SEQ ID NO: 12 - protein, 237 aa, Artificial sequence
  Synthetic polypeptide
MVSKGEETIMSVIKPDMKIKLRMEGNVNGHAFVIEGEGSGKPFEGIQTIDLEVKEGAPLPFAYDILTTAFHYGNRVFTKYPEDIPDYFKQSFPEGYSWERSMTYEDGGICIATNDITMEEDSFINKIHFKGTNFPPNGPVMQKRTVGWEVSTEKMYVRDGVLKGDVKMKLLLKGGSHYRCDFRTTYKVKQKAVKLPDYHFVDHRIEILSHDKDYNKVKLYEHAVARNSTDSMDELYK

SEQ ID NO: 13 - protein, 244 aa, Artificial sequence [sequence omitted]

SEQ ID NO: 14 - protein, 244 aa, Artificial sequence
  Synthetic polypeptide
MVSKGEELIKENMHMKLYMEGTVNNHHFKCTSEGEGKPYEGTQTGRIKVVEGGPLPFAFDILATCFMYGSKTFINHTQGIPDFFKQSFPEGFTWERVTTYEDGGVLTATQDTSLQDGCLIYNVKIRGVNFPSNGPVMQKKTLGWEASTETLYPADGGLEGRCDMALKLVGGGHLICNLKTTYRSKKPAKNLKMPGVYFVDRRLERIKEADNETYVEQHEVAVARYCDLPSKLGHKLNGMDELYK

SEQ ID NO: 15 - protein, 204 aa, Artificial sequence
  Synthetic polypeptide
NVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYK

SEQ ID NO: 16 - protein, 237 aa, Artificial sequence
  Synthetic polypeptide
NTEMMYPADGGLRGYTHMALKVDGGGHLSCSFVTTYRSKKTVGNIKMPGIHYVSHRLERLEESDNEMFVVQREHAVAKFVGLGGGGGTGGSMNSLIKENMRMKVVLEGSVNGHQFKCTGEGEGNPYMGTQTMRIKVIEGGPLPFAFDILATSFMSRTFIKYPKGIPDFFKQSFPEGFTWERVTRYEDGGVITVMQDTSLEDGCLVYHAQVRGVNFPSNGAVMQKKTKGWEPTRDQLT

SEQ ID NO: 17 - protein, 241 aa, Artificial sequence
  Synthetic polypeptide
VVSERMYPEDGALKSEIKKGLRLKDGGHYAAEVKTTYKAKKPVQLPGAYIVDIKLDIVSHNEDYTIVEQCERAEGRHSTGGMDELYKGGTGGSLVSKGEEDNMAIIKEFMRFKVHMEGSVNGHEFEIEGEGEGRPYEAFQTAKLKVTKGGPLPFAWDILSPQFMSKAYIKHPADIPDYFKLSFPEGFRWERVMNFEDGGIIHVNQDSSLQDGVFIYKVKLRGTNFPPDGPVMQKKTMGWEA

SEQ ID NO: 18 - protein, 230 aa, Artificial sequence
  Synthetic polypeptide
LECEKMYVRDGVLTGDIHMALLLEGNAHYRCDFRTTYKAKEKGVKLPGYHFVDHCIEILSHDKDYNKVKLYEHAVAHSGLPDNARRGGTGGSMVSAIKPDMKIKLRMEGNVNGHHFVIDGDGTGKPYEGKQTMDLEVKEGGPLPFAFDILTTAFHNRVFVKYPDNIQDYFKQSFPKGYSWERSMTFEDGGICYARNDITMEGDTFYNKVRFYGTNFPANGPVMQKKTLKW

SEQ ID NO: 19 - protein, 8 aa, Artificial sequence
  Synthetic polypeptide
  misc_feature (3)..(3): X is Y or F
  misc_feature (4)..(4): X is N or I
YNXXSHNV

SEQ ID NO: 20 - protein, 8 aa, Artificial sequence
  Synthetic polypeptide
  misc_feature (3)..(3): X is A, P, or V
  misc_feature (4)..(4): X is S, L, N, or T
  misc_feature (5)..(5): X is S, E, or T
  misc_feature (7)..(7): X is R, M, T, or K
  misc_feature (8)..(8): X is M or L
WEXXXEXX

SEQ ID NO: 21 - DNA, 2049 bp, Artificial sequence
  Synthetic polynucleotide
atgaagacgatcatcgccctgagctacatcttctgcctggtgttcgccgactacaaggacgatgatgacgccatggggcagccaggtaatggctctgcgttcttgttggccccgaacaggagccatgctcccgaccatgacgtcacccaacagagagatgaggtctgggtagtaggcatgggtattgtcatgtctctgatagtcttggcaatcgtgtttggaaatgtgctcgttatcacggcaatagctaagtttgagcgacttcaaacggtaacaaattatttcataacatctctcgcgtgtgcagatctcgtaatgggactcgctgtggtcccctttggcgcggcccatatcctgatgaagatgtggacttttggtaatttctggtgtgaattttggaccagcatagatgtactctgtgttacagcttcaattgaaactctctgtgtgatagccgttgatcgctatttcgccattacgtcccctgccaagtatcaatcattgcttaccaagaataaagcccgagtaataattctcatggtgtggatcgtaagcgggctcacatcttttttgccgattcagatgcactggtatagagcaacgcaccaagaagccataaactgctacgcaaatgaaacttgctgtgacttctttacaaatcaggcttacgctattgcctcttcaatagtcagtttttacgttcctctggttattatggtgtttgtatactcacgggtattccaggaggctaagcggcagctccagaaaatagacaagagtgagggacgctttcatgtgcagaatctttcagccgtcaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacaccagacaagttgaacaggacggacgcacaggtcatggcctcaggaggagttctaagttctgcttgaaggagcacaaagcgctgaagacgcttggaattatcatggggacgtttactctctgctggcttcctttcttcatagtaaacattgttcacgtaatccaagacaatctgattcgaaaggaggtgtatattctcctcaattggattgggtacgtaaacagcggatttaatcctcttatctattgccgaagccctgatttccgcatagcctttcaggaactgctttgtcttcgccgaagcagccttaaagcgtacggaaatggttacgctgcaaatgggaatacaggcgagcaaagcgggtatcacgtcgagcaagagaaggagaacaaacttctgtgcgaagacctgcctggcacggaagattttgtcggacaccaagggacggtaccgagtgacaatatcgacagtcaaggccgaaactgctcaactaatgattcactcctgtag

SEQ ID NO: 22 - protein, 685 aa, Artificial sequence
  Synthetic polypeptide
  misc_feature (163)..(163): Xaa can be any naturally occurring amino acid
MKTIIALSYIFCLVFADYKDDDDAMGQPGNGSAFLLAPNRSHAPDHDVTQQRDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPXKYQSLLTKNKARVIILMVWIVSGLTSFLPIQMHWYRATHQEAINCYANETCCDFFTNQAYAIASSIVSFYVPLVIMVFVYSRVFQEAKRQLQKIDKSEGRFHVQNLSAVNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNTRQVEQDGRTGHGLRRSSKFCLKEHKALKTLGIIMGTFTLCWLPFFIVNIVHVIQDNLIRKEVYILLNWIGYVNSGFNPLIYCRSPDFRIAFQELLCLRRSSLKAYGNGYAANGNTGEQSGYHVEQEKENKLLCEDLPGTEDFVGHQGTVPSDNIDSQGRNCSTNDSLLSTP

SEQ ID NO: 23 - DNA, 2010 bp, Artificial sequence
  Synthetic polynucleotide
  misc_feature (163)..(163): X is any amino acid or an amino acid selected from the group consisting of F, A, G, V, I, L, M, S, T
atgaagacgatcatcgccctgagctacatcttctgcctggtgttcgccgactacaaggacgatgatgacgccatggatagtagcgctgcgcctaccaacgcgtcaaactgcaccgatgctcttgcgtactcctcctgctccccggcacctagtcccggttcttgggtcaatttgtcccatctggacggaaacctctctgatccctgtgggcctaacaggacggacctcggtgggagggactccctttgcccgccgaccggatctccgtccatgataacggccattacaattatggcgttgtatagcatcgtatgcgttgtaggtctttttgggaatttcctggtgatgtacgtcatcgtcaggtacacaaagatgaaaacagctactaacatttatatatttaacctggcgctcgcggacgctctcgcaacgtcaacgctcccgtttcagtccgtgaattatctcatgggtacttggcctttcggaacaatactgtgtaaaattgttataagcatagattattataatatgttcacgtccatcttcacactctgcacaatgtctgtggataggtacattgctgtatgtcacccagctaaggcgcttgactttagaactccacgcaatgcaaagattataaatgtgtgcaactggatcttgtcctctgcaatagggcttcctgtgatgttcatggcgactactaagtacagacagggcagcatagattgcacactcaccttctcacacccaacttggtactgggaaaatctgctcaagatctgcgtcttcatttttgcttttatcatgccagtattgataatcacggtctgttacgggttgatgattttgcggctcaaatcagttcgaatgctcagtatcaaaaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacattatcggcagcaaggagaaggaccgcaacctcagaaggataacgagaatggtgctggtcgtagtggcggttttcattgtttgttggacgccaatacacatatacgtgattataaaggctctggtgacaattcccgaaacaacgtttcagacggtctcttggcatttctgtattgcattggggtacactaattcctgccttaatcctgtattgtacgcctttctggatgaaaactttaaaagatgtttccgcgagttctgcataccgaccagcagcaacattgaacaacaaaactccacgcgcatacggcaaaatactagggatcacccgtccactgcgaatactgtagaccgaacgaaccatcagttggagaatttggaagcggaaactgctcctctgccatga

SEQ ID NO: 24 - protein, 669 aa, Artificial sequence
  Synthetic polypeptide
  misc_feature (199)..(199): X is any amino acid or an amino acid selected from the group consisting of F, A, G, V, I, L, M, S, T
MKTIIALSYIFCLVFADYKDDDDAMDSSAAPTNASNCTDALAYSSCSPAPSPGSWVNLSHLDGNLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPXKALDFRTPRNAKIINVCNWILSSAIGLPVMFMATTKYRQGSIDCTLTFSHPTWYWENLLKICVFIFAFIMPVLIITVCYGLMILRLKSVRMLSIKNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNIIGSKEKDRNLRRITRMVLVVVAVFIVCWTPIHIYVIIKALVTIPETTFQTVSWHFCIALGYTNSCLNPVLYAFLDENFKRCFREFCIPTSSNIEQQNSTRIRQNTRDHPSTANTVDRTNHQLENLEAETAPLP

SEQ ID NO: 25 - DNA, 1962 bp, Artificial sequence
  Synthetic polynucleotide
atgaagacgatcatcgccctgagctacatcttctgcctggtgttcgccgactacaaggacgatgatgacgccatgaggactctgaacacctctgccatggacgggactgggctggtggtggagagggacttctctgttcgtatcctcactgcctgtttcctgtcgctgctcatcctgtccacgctcctggggaacacgctggtctgtgctgccgttatcaggttccgacacctgcggtccaaggtgaccaacttctttgtcatctccttggctgtgtcagatctcttggtggccgtcctggtcatgccctggaaggcagtggctgagattgctggcttctggccctttgggtccttctgtaacatctgggtggcctttgacatcatgtgctccactgcatccatcctcaacctctgtgtgatcagcgtggacaggtattgggctatctccagccctgcccggtatgagagaaagatgacccccaaggcagccttcatcctgatcagtgtggcatggaccttgtctgtactcatctccttcatcccagtgcagctcagctggcacaaggcaaaacccacaagcccctctgatggaaatgccacttccctggctgagaccatagacaactgtgactccagcctcagcaggacatatgccatctcatcctctgtaatcagcttttacatccctgtggccatcatgattgtcacctacaccaggatctacaggattgctcagaaacaaatacggcgcattgcggccttggagagggcagcagtccacgccaagaattgctctcggaacgtgtatatcaaggctgataaacaaaagaatggtatcaaagctaatttcaaaatccgccacaatatcgaagatggcggcgtccagctcgcttatcattatcagcagaatacacctatcggtgacgggccggtgcttttgcctgataaccattacctgagtgttcaaagtaaactgagcaaggatccaaatgaaaaaagggaccacatggtgcttctcgaatttgtaacggctgcaggcattactctcgggatggacgaactttacaaaggagggaccggaggcagcatggtgtccaagggggaggaacttttcactggcgtcgtgccgatactcgtcgaactcgatggagatgttaatggacacaaattttcagtcagtggcgaaggggaaggggatgctacttacgggaaactcacactgaagtttatttgtacgacaggcaaactcccggtaccttggccgaccttggtgaccacgttgacgtatggagtacagtgcttctccaggtacccggaccacatgaagcaacatgactttttcaaaagcgctatgcccgagggctacattcaagaacggactattttctttaaggacgatggaaactataaaaccagagctgaggtgaaattcgagggtgacactcttgtaaaccggatagaactcaaaggtatagatttcaaagaagacggaaacatcttggggcataaactcgagtataatcctcctcagaccaccacaggtaatggaaagcctgtcgaatgttctcaaccggaaagttcttttaagatgtccttcaaaagagaaactaaagtcctgaagactctgtcggtgatcatgggtgtgtttgtgtgctgttggctacctttcttcatcttgaactgcattttgcccttctgtgggtctggggagacgcagcccttctgcattgattccaacacctttgacgtgtttgtgtggtttgggtgggctaattcatccttgaaccccatcatttatgcctttaatgctgattttcggaaggcattttcaaccctcttaggatgctacagactttgccctgcgacgaataatgccatagagacggtgagtatcaataacaatggggccgcgatgttttccagccatcatgagccattctgctacgagaatgaagtctga

SEQ ID NO: 26 - protein, 653 aa, Artificial sequence
  Synthetic polypeptide
  misc_feature (153)..(153): X is any amino acid or an amino acid selected from the group consisting of F, A, G, V, I, L, M, S, T
MKTIIALSYIFCLVFADYKDDDDAMRTLNTSAMDGTGLVVERDFSVRILTACFLSLLILSTLLGNTLVCAAVIRFRHLRSKVTNFFVISLAVSDLLVAVLVMPWKAVAEIAGFWPFGSFCNIWVAFDIMCSTASILNLCVISVDRYWAISSPXRYERKMTPKAAFILISVAWTLSVLISFIPVQLSWHKAKPTSPSDGNATSLAETIDNCDSSLSRTYAISSSVISFYIPVAIMIVTYTRIYRIAQKQIRRIAALERAAVHAKNCSRNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNPPQTTTGNGKPVECSQPESSFKMSFKRETKVLKTLSVIMGVFVCCWLPFFILNCILPFCGSGETQPFCIDSNTFDVFVWFGWANSSLNPIIYAFNADFRKAFSTLLGCYRLCPATNNAIETVSINNNGAAMFSSHHEPFCYENEV

SEQ ID NO: 27 - DNA, 2223 bp, Artificial sequence
  Synthetic polypeptide
atgaagacgatcatcgccctgagctacatcttctgcctggtgttcgccgactacaaggacgatgatgacgccatggacatactttgtgaagagaatacttcactctcttctactactaactctcttatgcaactgaacgatgatacccgattgtactcaaacgacttcaattccggcgaagcgaacaccagtgacgcattcaactggactgtcgattctgaaaacagaactaatctgtcatgcgagggttgtcttagtccctcttgtctcagcctgttgcacctccaggaaaagaactggtcagcactgctcactgcggtagtgataatactcactattgctggcaatattctcgtaattatggcagtctccttggagaagaaactccaaaacgccacaaattattttcttatgagccttgccatcgcagatatgctcttgggatttttggtgatgcctgtgagtatgctcacgatactgtatggatatcgctggcctctgccgtctaaactttgcgctgtgtggatttacttggatgtccttttttcaactgcgagtattatgcatctttgcgccattagtcttgataggtatgtggctatccaaaatcctgcccaccattcccgctttaatagtagaactaaggcttttctgaaaataatagcagtgtggaccatatctgtcggcataagcatgcctatccccgtatttggacttcaagatgactcaaaggtattcaaagaagggtcatgtctgctggccgatgacaatttcgtgcttattggatccttcgtcagtttcttcattcctttgacaatcatggtgattacctactttcttacgattaaatctttgcaaaaggaggctactctgtgcgtcagcgacctcggcactcgggccaaatctcggaacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaaccttttccttgccagcttctcattccttccccagtcctctctttccagtgagaaacttttccaacgatccatacatagggagccgggtagttatacaggacggcggacgatgcaatcaattagtaatgagcaaaaggcttgtaaggtactcggcatagtcttctttctgtttgtggtgatgtggtgtcccttctttataacgaatatcatggcagtgatctgcaaggaatcatgcaatgaggatgtgatcggggcacttctgaacgttttcgtgtggatagggtatctgtcaagtgctgtgaacccactggtctataccttgtttaataagacataccgctcagccttttcacggtatattcaatgtcagtataaggaaaacaagaaacctctgcaacttattcttgtgaacactatccctgccctggcttataagtcatcacagttgcagatgggccagaaaaaaaattccaagcaggacgcgaagacaacagacaacgattgtagtatggttgccctcggcaagcagcacagtgaagaagcgagcaaagacaatagtgatggcgtaaacgaaaaagtcagttgtgtataa

SEQ ID NO: 28 - protein, 740 aa, Artificial sequence
  Synthetic polypeptide
  misc_feature (205)..(205): X is any amino acid or an amino acid selected from the group consisting of F, A, G, V, I, L, M, S, T
MKTIIALSYIFCLVFADYKDDDDAMDILCEENTSLSSTTNSLMQLNDDTRLYSNDFNSGEANTSDAFNWTVDSENRTNLSCEGCLSPSCLSLLHLQEKNWSALLTAVVIILTIAGNILVIMAVSLEKKLQNATNYFLMSLAIADMLLGFLVMPVSMLTILYGYRWPLPSKLCAVWIYLDVLFSTASIMHLCAISLDRYVAIQNPXHHSRFNSRTKAFLKIIAVWTISVGISMPIPVFGLQDDSKVFKEGSCLLADDNFVLIGSFVSFFIPLTIMVITYFLTIKSLQKEATLCVSDLGTRAKSRNVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNLFLASFSFLPQSSLSSEKLFQRSIHREPGSYTGRRTMQSISNEQKACKVLGIVFFLFVVMWCPFFITNIMAVICKESCNEDVIGALLNVFVWIGYLSSAVNPLVYTLFNKTYRSAFSRYIQCQYKENKKPLQLILVNTIPALAYKSSQLQMGQKKNSKQDAKTTDNDCSMVALGKQHSEEASKDNSDGVNEKVSCV

SEQ ID NO: 29 - DNA, 613 bp, Artificial sequence
  Synthetic polynucleotide
aacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagc

SEQ ID NO: 30 - protein, 662 aa, Artificial sequence
  Synthetic polypeptide
  misc_feature (129)..(129): X is any amino acid or an amino acid selected from the group consisting of F, A, G, V, I, L, M, S, T
  misc_feature (648)..(648): X is L or M
MRTLNTSAMDGTGLVVERDFSVRILTACFLSLLILSTLLGNTLVCAAVIRFRHLRSKVTNFFVISLAVSDLLVAVLVMPWKAVAEIAGFWPFGSFCNIWVAFDIMCSTASILNLCVISVDRYWAISSPXRYERKMTPKAAFILISVAWTLSVLISFIPVQLSWHKAKPTSPSDGNATSLAETIDNCDSSLSRTYAISSSVISFYIPVAIMIVTYTRIYRIAQKQLQKIDLSSLINVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNNHDQLKRETKVLKTLSVIMGVFVCCWLPFFILNCILPFCGSGETQPFCIDSNTFDVFVWFGWANSSLNPIIYAFNADFRKAFSTLLGCYRLCPATNNAIETVSINNNGAAMFSSHHEPRGSISKECNLVYLIPHAVGSSEDLKKEEAAGIARPLEKLSPALSVILDYDTDVSXEKIQPITQNGQHPT

SEQ ID NO: 31 - protein, 670 aa, Artificial sequence
  Synthetic polypeptide
  misc_feature (164)..(164): X is any amino acid or an amino acid selected from the group consisting of F, A, G, V, I, L, M, S, T
MGAGVLVLGASEPGNLSSAAPLPDGAATAARLLVPASPPASLLPPASESPEPLSQQWTAGMGLLMALIVLLIVAGNVLVIVAIAKTPRLQTLTNLFIMSLASADLVMGLLVVPFGATIVVWGRWEYGSFFCELWTSVDVLCVTASIETLCVIALDRYLAITSPXRYQSLLTRARARGLVCTVWAISALVSFLPILMHWWRAESDEARRCYNDPKCCDFVTNRAYAIASSVVSFYVPLCIMAFVYLRVFREAQKQLQKIDLSSLINVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNNHDQLREQKALKTLGIIMGVFTLCWLPFFLANVVKAFHRELVPDRLFVFFNWLGYANSAFNPIIYCRSPDFRKAFQGLLCCARRAARRRHATHGDRPRASGCLARPGPPPSPGAASDDDDDDVVGATPPARLLEPWAGCNGGAAADSDSSLDEPCRPGFASESKV

SEQ ID NO: 32 - protein, 629 aa, Artificial sequence
  Synthetic polypeptide
  misc_feature (139)..(139): X is any amino acid or an amino acid selected from the group consisting of F, A, G, V, I, L, M, S, T
MGQPGNGSAFLLAPNRSHAPDHDVTQQRDEVWVVGMGIVMSLIVLAIVFGNVLVITAIAKFERLQTVTNYFITSLACADLVMGLAVVPFGAAHILMKMWTFGNFWCEFWTSIDVLCVTASIETLCVIAVDRYFAITSPXKYQSLLTKNKARVIILMVWIVSGLTSFLPIQMHWYRATHQEAINCYANETCCDFFTNQAYAIASSIVSFYVPLVIMVFVYSRVFQEAKRQLQKIDLSSLINVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNNHDQLKEHKALKTLGIIMGTFTLCWLPFFIVNIVHVIQDNLIRKEVYILLNWIGYVNSGFNPLIYCRSPDFRIAFQELLCLRRSRYPNVRPNNGYIYNAHSWQSENREQSKGSSGDSDHAEGNLAKEECLSADKTDSNGNCSKAQMRVL

SEQ ID NO: 33 - protein, 675 aa, Artificial sequence
  Synthetic polypeptide
  misc_feature (181)..(181): X is any amino acid or an amino acid selected from the group consisting of F, A, G, V, I, L, M, S, T
MDILCEENTSLSSTTNSLMQLNDDTRLYSNDFNSGEANTSDAFNWTVDSENRTNLSCEGCLSPSCLSLLHLQEKNWSALLTAVVIILTIAGNILVIMAVSLEKKLQNATNYFLMSLAIADMLLGFLVMPVSMLTILYGYRWPLPSKLCAVWIYLDVLFSTASIMHLCAISLDRYVAIQNPXHHSRFNSRTKAFLKIIAVWTISVGISMPIPVFGLQDDSKVFKEGSCLLADDNFVLIGSFVSFFIPLTIMVITYFLTIKSLQKQLQKIDLSSLINVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNNHDQLNEQKACKVLGIVFFLFVVMWCPFFITNIMAVICKESCNEDVIGALLNVFVWIGYLSSAVNPLVYTLFNKTYRSAFSRYIQCQYKENKKPLQLILVNTIPALAYKSSQLQMGQKKNSKQDAKTTDNDCSMVALGKQHSEEASKDNSDGVNEKVSCV

SEQ ID NO: 34 - protein, 649 aa, Artificial sequence
  Synthetic polypeptide
  misc_feature (110)..(110): X is any amino acid or an amino acid selected from the group consisting of F, A, G, V, I, L, M, S, T
MPIMGSSVYITVELAIAVLAILGNVLVCWAVWLNSNLQNVTNYFVVSLAAADIAVGVLAIPFAITISTGFCAACHGCLFIACFVLVLTQSSIFSLLAIAIDRYIAIRIPXRYNGLVTGTRAKGIIAICWVLSFAIGLTPMLGWNNCGQPKEGKNHSQGCGEGQVACLFEDVVPMNYMVYFNFFACVLVPLLLMLGVYLRIFLAARRQLQKIDLSSLINVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNNHDQLKEVHAAKSLAIIVGLFALCWLPLHIINCFTFFCPDCSHAPLWLMYLAIVLSHTNSVVNPFIYAYRIREFRQTFRKIIRSHVLRQQEPFKAAGTSARVLAAHGSDGEQVSLRLNGHPPGVWANGSAPHPERRPNGYALGLVSGGSAQESQGNTGLPDVELLSHELKGVCPEPPGLDDPLAQDGAGVS

SEQ ID NO: 35 - protein, 565 aa, Artificial sequence
  Synthetic polypeptide
  misc_feature (139)..(139): X is any amino acid or an amino acid selected from the group consisting of F, A, G, V, I, L, M, S, T
MGSLQPDAGNASWNGTEAPGGGARATPYSLQVTLTLVCLAGLLMLLTVFGNVLVIIAVFTSRALKAPQNLFLVSLASADILVATLVIPFSLANEVMGYWYFGKAWCEIYLALDVLFCTSSIVHLCAISLDRYWSITQAXEYNLKRTPRRIKAIIITVWVISAVISFPPLISIEKKGGGGGPQPAEPRCEINDQKWYVISSCIGSFFAPCLIMILVYVRIYQIAKRQLQKIDLSSLINVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNNHDQLREKRFTFVLAVVIGVFVVCWFPFFFTYTLTAVGCSVPRTLFKFFFWFGYCNSSLNPVIYTIFNHDFRRAFKKILCRGDRKRIV

SEQ ID NO: 36 - protein, 630 aa, Artificial sequence
  Synthetic polypeptide
  misc_feature (164)..(164): X is any amino acid or an amino acid selected from the group consisting of F, A, G, V, I, L, M, S, T
MDSPIQIFRGEPGPTCAPSACLPPNSSAWFPGWAEPDSNGSAGSEDAQLEPAHISPAIPVIITAVYSVVFVVGLVGNSLVMFVIIRYTKMKTATNIYIFNLALADALVTTTMPFQSTVYLMNSWPFGDVLCKIVISIDYYNMFTSIFTLTMMSVDRYIAVCHPXKALDFRTPLKAKIINICIWLLSSSVGISAIVLGGTKVREDVDVIECSLQFPDDDYSWWDLFMKICVFIFAFVIPVLIIIVCYTLMILRLKSQLQKIDLSSLINVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNNHDQLREKDRNLRRITRLVLVVVAVFVVCWTPIHIFILVEALGSTSHSTAALAAYYFCIALGYTNAALNPILYAFLDENFKRCFRDFCFPLKMRMERQATARVRNTVQDPAYLRDIDGMNKPV

SEQ ID NO: 37 - protein, 650 aa, Artificial sequence
  Synthetic polypeptide
  misc_feature (175)..(175): X is any amino acid or an amino acid selected from the group consisting of F, A, G, V, I, L, M, S, T
MDSSAAPTNASNCTDALAYSSCSPAPSPGSWVNLSHLDGNLSDPCGPNRTDLGGRDSLCPPTGSPSMITAITIMALYSIVCVVGLFGNFLVMYVIVRYTKMKTATNIYIFNLALADALATSTLPFQSVNYLMGTWPFGTILCKIVISIDYYNMFTSIFTLCTMSVDRYIAVCHPXKALDFRTPRNAKIINVCNWILSSAIGLPVMFMATTKYRQGSIDCTLTFSHPTWYWENLLKICVFIFAFIMPVLIITVCYGLMILRLKSQLQKIDLSSLINVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNNHDQLKEKDRNLRRITRMVLVVVAVFIVCWTPIHIYVIIKALVTIPETTFQTVSWHFCIALGYTNSCLNPVLYAFLDENFKRCFREFCIPTSSNIEQQNSTRIRQNTRDHPSTANTVDRTNHQLENLEAETAPLP

SEQ ID NO: 38 - protein, 585 aa, Artificial sequence
  Synthetic polypeptide
  misc_feature (154)..(154): X is any amino acid or an amino acid selected from the group consisting of F, A, G, V, I, L, M, S, T
MEPAPSAGAELQPPLFANASDAYPSACPSAGANASGPPGARSASSLALAIAITALYSAVCAVGLLGNVLVMFGIVRYTKMKTATNIYIFNLALADALATSTLPFQSAKYLMETWPFGELLCKAVLSIDYYNMFTSIFTATMMSVDRYIAVCHPXKALDFRTPAKAKLINICIWVLASGVGVPIMVMAVTRPRDGAVVCMLQFPSPSWYWDTVTKICVFLFAFVVPILIITVCYGLMLLRLRSQLQKIDLSSLINVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNNHDQLKEKDRSLRRITRMVLVVVGAFVVCWAPIHIFVIVWTLVDIDRRDPLVVAALHLCIALGYANSSLNPVLYAFLDENFKRCFRQLCRA

SEQ ID NO: 39 - protein, 609 aa, Artificial sequence
  Synthetic polypeptide
  misc_feature (146)..(146): X is any amino acid or an amino acid selected from the group consisting of F, A, G, V, I, L, M, S, T
MSENGSFANCCEAGGWAVRPGWSGAGSARPSRTPRPPWVAPALSAVLIVTTAVDVVGNLLVILSVLRNRKLRNAGNLFLVSLALADLVVAFYPYPLILVAIFYDGWALGEEHCKASAFVMGLSVIGSVFNITAIAINRYCYICHSXAYHRIYRRWHTPLHICLIWLLTVVALLPNFFVGSLEYDPRIYSCTFIQTASTQYTAAVVVIHFLLPIAVVSFCYLRIWVLVLQARRQLQKIDLSSLINVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNNHDQLKPSDLRSFLTMFVVFVIFAICWAPLNCIGLAVAINPQEMAPQIPEGLFVTSYLLAYFNSCLNAIVYGLLNQNFRREYKRILLALWNPRHCIQDASKGSHAEGLQSPAPPIIGVQHQADAL

SEQ ID NO: 40 - protein, 692 aa, Artificial sequence
  Synthetic polypeptide
  misc_feature (222)..(222): X is any amino acid or an amino acid selected from the group consisting of F, A, G, V, I, L, M, S, T
MKSILDGLADTTFRTITTDLLYVGSNDIQYEDIKGDMASKLGYFPQKFPLTSFRGSPFQEKMTAGDNPQLVPADQVNITEFYNKSLSSFKENEENIQCGENFMDIECFMVLNPSQQLAIAVLSLTLGTFTVLENLLVLCVILHSRSLRCRPSYHFIGSLAVADLLGSVIFVYSFIDFHVFHRKDSRNVFLFKLGGVTASFTASVGSLFLTAIDRYISIHRPXAYKRIVTRPKAVVAFCLMWTIAIVIAVLPLLGWNCEKLQSVCSDIFPHIDETYLMFWIGVTSVLLLFIVYAYMYILWKAHSHAVRMIQRQLQKIDLSSLINVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNNHDQLRMDIRLAKTLVLILVVLIICWGPLLAIMVYDVFGKMNKLIKTVFAFCSMLCLLNSTVNPIIYALRSKDLRHAFRSMFPSCEGTAQPLDNSMGDSDCLHKHANNAASVHRAAESCIKSTVKIAKV

SEQ ID NO: 41 - protein, 554 aa, Artificial sequence
  Synthetic polypeptide
  misc_feature (133)..(133): X is any amino acid or an amino acid selected from the group consisting of F, A, G, V, I, L, M, S, T
MSLPNSSCLLEDKMCEGNKTTMASPQLMPLVVVLSTICLVTVGLNLLVLYAVRSERKLHTVGNLYIVSLSVADLIVGAVVMPMNILYLLMSKWSLGRPLCLFWLSMDYVASTASIFSVFILCIDRYRSVQQPXRYLKYRTKTRASATILGAWFLSFLWVIPILGWNHFMQQTSVRREDKCETDFYDVTWFKVMTAIINFYLPTLLMLWFYAKIYKAVRQLQKIDLSSLINVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNNHDQLRERKAAKQLGFIMAAFILCWIPYFIFFMVIAFCKNCCNEHLHMFTIWLGYINSTLNPLIYPLCNENFKKTFKRILHIRS

SEQ ID NO: 42 - protein, 627 aa, Artificial sequence
  Synthetic polypeptide
MNSTLFSQVENHSVHSNFSEKNAQLLAFENDDCHLPLAMIFTLALAYGAVIILGVSGNLALIIIILKQKEMRNVTNILIVNLSFSDLLVAIMCLPFTFVYTLMDHWVFGEAMCKLNPFVQCVSITVSIFSLVLIAVERHQLIINPRGWRPNNRHAYVGIAVIWVLAVASSLPFLIYQVMTDEPFQNVTLDAYKDKYVCFDQFPSDSHRLSYTTLLLVLQYFGPLCFIFICYFKIYIRLKRRQLQKIDLSSLINVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNNHDQLSETKRINIMLLSIVVAFAVCWLPLTIFNTVFDWNHQIIATCNHNLLFLLCHLTAMISTCVNPIFYGFLNKNFQRDLQFFFNFCDFRSRDDDYETIAMSTMHTDVSKTSLKQASPVAFKKINNNDDNEKI

SEQ ID NO: 43 - protein, 558 aa, Artificial sequence
  Synthetic polypeptide
  misc_feature (129)..(129): X is any amino acid or an amino acid selected from the group consisting of F, A, G, V, I, L, M, S, T
MNNSTNSSNNSLALTSPYKTFEVVFIVLVAGSLSLVTIIGNILVMVSIKVNRHLQTVNNYFLFSLACADLIIGVFSMNLYTLYTVIGYWPLGPVVCDLWLALDYVVSNASVMNLLIISFDRYFCVTKPXTYPVKRTTKMAGMMIAAAWVLSFILWAPAILFWQFIVGVRTVEDGECYIQFFSNAAVTFGTAIAAFYLPVIIMTVLYWHISRASKSQLQKIDLSSLINVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNNHDQLREKKVTRTILAILLAFIITWAPYNVMVLINTFCAPCIPNTVWTIGYWLCYINSTINPACYALCNATFKKTFKHLLMCHYKNIGATR

SEQ ID NO: 44 - protein, 637 aa, Artificial sequence
  Synthetic polypeptide
  misc_feature (152)..(152): X is any amino acid or an amino acid selected from the group consisting of F, A, G, V, I, L, M, S, T
MEPSATPGAQMGVPPGSREPSPVPPDYEDEFLRYLWRDYLYPKQYEWVLIAAYVAVFVVALVGNTLVCLAVWRNHHMRTVTNYFIVNLSLADVLVTAICLPASLLVDITESWLFGHALCKVIPYLQAVSVSVAVLTLSFIALDRWYAICHPXLFKSTARRARGSILGIWAVSLAIMVPQAAVMECSSVLPELANRTRLFSVCDERWADDLYPKIYHSCFFIVTYLAPLGLMAMAYFQIFRKLWGRQLQKIDLSSLINVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNNHDQLRARRKTAKMLMVVLLVFALCYLPISVLNVLKRVFGMFRQASDREAVYACFTFSHWLVYANSAANPIIYNFLSGKFREQFKAAFSCCLPGLGPCGSLKAPSPRSSASHKSLSLQSRCSISKISEHVVLTSVTTVLP

SEQ ID NO: 45 - DNA, 1677 bp, Artificial sequence
  Synthetic polynucleotide
atggacccccttaacctctcatggtacgacgatgatcttgagaggcagaactggtcccgaccattcaatgggtctgatggtaaggctgaccggcctcattacaattattatgcgaccctgcttactcttcttatcgctgtgatcgtattcggcaacgtcttggtttgcatggcagtctctagggaaaaagcgctccagacgacaactaattacttgattgtgagtctggctgtagctgacttgcttgtggcgaccctggtgatgccatgggtcgtatacttggaagtcgttggcgagtggaagttttctaggattcattgcgacatatttgtaactctggacgtaatgatgtgtactgcttccattttgaacctctgcgctatatccattgacaggtacacggcggttgctatgccgatgctttataatacccggtattcaagcaaaaggcgagtaactgtgatgataagcattgtatgggtgctcagtttcacaattagctgccctctgctcttcggccttaacaacgcggatcaaaatgaatgcatcatcgcaaacccggcttttgtggtttatagcagcattgttagcttctatgtgccattcatagttacgctccttgtttatataaaaatttatatcgtgcttaggcgccgccgaaaacgagttaacaccaagcggagcagcctgagctcactcattaatgtatatatcaaagctgataagcaaaaaaacggtatcaaggctaattttaagatcagacataatatagaggatggaggcgttcaactggcctaccactaccagcaaaacacgccgatcggggatgggccagtacttctgccagataaccattatctctcagttcaaagcaaactctctaaggaccctaatgagaaacgagatcatatggttctgctcgaattcgttacagccgccggtatcacacttgggatggacgagttgtataagggtggaacaggagggtcaatggtaagcaaaggcgaggagctgtttacgggggtcgtcccgatacttgttgaactcgacggcgatgtcaacgggcacaaattctcagtgagtggcgagggggaaggagacgccacttatggaaaactgacattgaaattcatatgtacgactgggaagttgcctgtgccttggcctacgctcgttactacacttacttacggggtacagtgtttcagtaggtatccagatcacatgaaacagcacgattttttcaagagtgcaatgccggaaggatatatacaagaaagaactattttctttaaagatgacggcaactataaaacgcgagcagaggtgaagtttgagggcgataccttggttaataggatcgaactcaaaggcatagacttcaaagaagacggaaacattctgggtcacaaactggaatacaacaatcatgaccaactgcagaaggaaaagaaggccacgcaaatgttggcaatcgtgctcggcgtgttcataatctgctggcttccattttttataacgcatatattgaacatacactgtgattgcaatattccaccagtcctgtatagtgcgtttacgtggttgggttatgtgaattctgcggttaacccgatcatttacaccacgttcaacatagaattccgaaaggcattcctcaaaatattgcattgttag

SEQ ID NO: 46 - protein, 550 aa, Artificial sequence
  Synthetic polypeptide
  misc_feature (140)..(140): X is any amino acid or an amino acid selected from the group consisting of F, A, G, V, I, L, M, S, T
MDPLNLSWYDDDLERQNWSRPFNGSDGKADRPHYNYYATLLTLLIAVIVFGNVLVCMAVSREKALQTTTNYLIVSLAVADLLVATLVMPWVVYLEVVGEWKFSRIHCDIFVTLDVMMCTASILNLCAISIDRYTAVAMPXLYNTRYSSKRRVTVMISIVWVLSFTISCPLLFGLNNADQNECIIANPAFVVYSSIVSFYVPFIVTLLVYIKIYIVLRRRRKLSSLINVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNNHDQLQKEKKATQMLAIVLGVFIICWLPFFITHILNIHCDCNIPPVLYSAFTWLGYVNSAVNPIIYTTFNIEFRKAFLKILHC

SEQ ID NO: 47 - DNA, 1671 bp, Artificial sequence
  Synthetic polynucleotide
atggggaacagatccactgcagatgcagacggtcttctcgcaggccggggacctgctgccggagcgagcgctggggcttccgcaggtcttgctgggcagggggccgcggccttggttggaggcgttttgcttataggggccgttcttgctggcaatagtttggtatgtgtttcagttgcgacagagcgcgcacttcagacgccgactaactcctttatagtgagtttggctgctgcagatctcttgttggcattgttggtactcccactgttcgtttattcagaagtacagggtggcgcatggctcctgtcacccaggttgtgtgatgccttgatggccatggatgttatgctgtgtaccgcttctatctttaacctttgtgctatcagtgttgacagattcgtcgcggtcgcggtccctctgaggtataaccggcaaggaggcagcaggaggcaactgctgctgatcggcgcaacttggctcctctccgcagcagtggccgcgcctgttctgtgtggtctcaacgacgttcgcggcagagacccggctgtatgtcgcctcgaggatagagattatgtcgtatactcaagtgtgtgttccttttttcttccttgcccactgatgcttctgttgtattgggctacctttagaggactgcaacgctgggaagtcctgagctcactcattaatgtatatatcaaagctgataagcaaaaaaacggtatcaaggctaattttaagatcagacataatatagaggatggaggcgttcaactggcctaccactaccagcaaaacacgccgatcggggatgggccagtacttctgccagataaccattatctctcagttcaaagcaaactctctaaggaccctaatgagaaacgagatcatatggttctgctcgaattcgttacagccgccggtatcacacttgggatggacgagttgtataagggtggaacaggagggtcaatggtaagcaaaggcgaggagctgtttacgggggtcgtcccgatacttgttgaactcgacggcgatgtcaacgggcacaaattctcagtgagtggcgagggggaaggagacgccacttatggaaaactgacattgaaattcatatgtacgactgggaagttgcctgtgccttggcctacgctcgttactacacttacttacggggtacagtgtttcagtaggtatccagatcacatgaaacagcacgattttttcaagagtgcaatgccggaaggatatatacaagaaagaactattttctttaaagatgacggcaactataaaacgcgagcagaggtgaagtttgagggcgataccttggttaataggatcgaactcaaaggcatagacttcaaagaagacggaaacattctgggtcacaaactggaatacaacaatcatgaccaactgggccgcgaacggaaagccatgcgagttttgccggtggtagtaggggcattccttctttgttggacccctttttttgtggtgcatataacgcaggctctgtgcccggcctgttctgtcccaccccgcctcgtgtcagctgtcacttggttgggttacgtaaactcagccctcaatccagttatctatacggttttcaatgccgagttcaggaatgtttttaggaaggcccttagagcctgttgttag

SEQ ID NO: 48 - protein, 556 aa, Artificial sequence
  Synthetic polypeptide
  misc_feature (141)..(141): X is any amino acid or an amino acid selected from the group consisting of F, A, G, V, I, L, M, S, T
MGNRSTADADGLLAGRGPAAGASAGASAGLAGQGAAALVGGVLLIGAVLAGNSLVCVSVATERALQTPTNSFIVSLAAADLLLALLVLPLFVYSEVQGGAWLLSPRLCDALMAMDVMLCTASIFNLCAISVDRFVAVAVPXRYNRQGGSRRQLLLIGATWLLSAAVAAPVLCGLNDVRGRDPAVCRLEDRDYVVYSSVCSFFLPCPLMLLLYWATFRGLQRWEVLSSLINVYIKADKQKNGIKANFKIRHNIEDGGVQLAYHYQQNTPIGDGPVLLPDNHYLSVQSKLSKDPNEKRDHMVLLEFVTAAGITLGMDELYKGGTGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYIQERTIFFKDDGNYKTRAEVKFEGDTLVNRIELKGIDFKEDGNILGHKLEYNNHDQLGRERKAMRVLPVVVGAFLLCWTPFFVVHITQALCPACSVPPRLVSAVTWLGYVNSALNPVIYTVFNAEFRNVFRKALRACC

SEQ ID NO: 49 - protein, 10 aa, Artificial sequence
  Synthetic polypeptide
  misc_feature (5)..(6): cpGFP linker
LSSGYMHDQL

SEQ ID NO: 50 - protein, 10 aa, Artificial sequence
  Synthetic polypeptide
  misc_feature (5)..(6): cpGFP linker
LSSLINHDQL

SEQ ID NO: 51 - protein, 10 aa, Artificial sequence
  Synthetic polypeptide
  misc_feature (4)..(4): Xaa can be any naturally occurring amino acid
  misc_feature (5)..(6): cpGFP linker
  misc_feature (7)..(7): Xaa can be any naturally occurring amino acid
LSSXXXXDQL

SEQ ID NO: 52 - DNA, 2085 bp, Artificial sequence
  Synthetic polynucleotide
atgaagacgatcatcgccctgagctacatcttctgcctggtgttcgccgactacaaggacgatgatgacgccatggacatactttgtgaagagaatacttcactctcttctactactaactctcttatgcaactgaacgatgatacccgattgtactcaaacgacttcaattccggcgaagcgaacaccagtgacgcattcaactggactgtcgattctgaaaacagaactaatctgtcatgcgagggttgtcttagtccctcttgtctcagcctgttgcacctccaggaaaagaactggtcagcactgctcactgcggtagtgataatactcactattgctggcaatattctcgtaattatggcagtctccttggagaagaaactccaaaacgccacaaattattttcttatgagccttgccatcgcagatatgctcttgggatttttggtgatgcctgtgagtatgctcacgatactgtatggatatcgctggcctctgccgtctaaactttgcgctgtgtggatttacttggatgtccttttttcaactgcgagtattatgcatctttgcgccattagtcttgataggtatgtggctatccaaaatcctgctcaccattcccgctttaatagtagaactaaggcttttctgaaaataatagcagtgtggaccatatctgtcggcataagcatgcctatccccgtatttggacttcaagatgactcaaaggtattcaaagaagggtcatgtctgctggccgatgacaatttcgtgcttattggatccttcgtcagtttcttcattcctttgacaatcatggtgattacctactttcttacgattaaatctttgcaaaagcagctgagctcaggttataacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaacatgcatgaccaactgaatgagcaaaaggcttgtaaggtactcggcatagtcttctttctgtttgtggtgatgtggtgtcccttctttataacgaatatcatggcagtgatctgcaaggaatcatgcaatgaggatgtgatcggggcacttctgaacgttttcgtgtggatagggtatctgtcaagtgctgtgaacccactggtctataccttgtttaataagacataccgctcagccttttcacggtatattcaatgtcagtataaggaaaacaagaaacctctgcaacttattcttgtgaacactatccctgccctggcttataagtcatcacagttgcagatgggccagaaaaaaaattccaagcaggacgcgaagacaacagacaacgattgtagtatggttgccctcggcaagcagcacagtgaagaagcgagcaaagacaatagtgatggcgtaaacgaaaaagtcagttgtgtataa

SEQ ID NO: 53 - DNA, 2112 bp, Artificial sequence
  Synthetic polynucleotide
atgaagacgatcatcgccctgagctacatcttctgcctggtgttcgccgactacaaggacgatgatgacgccatggacatactttgtgaagagaatacttcactctcttctactactaactctcttatgcaactgaacgatgatacccgattgtactcaaacgacttcaattccggcgaagcgaacaccagtgacgcattcaactggactgtcgattctgaaaacagaactaatctgtcatgcgagggttgtcttagtccctcttgtctcagcctgttgcacctccaggaaaagaactggtcagcactgctcactgcggtagtgataatactcactattgctggcaatattctcgtaattatggcagtctccttggagaagaaactccaaaacgccacaaattattttcttatgagccttgccatcgcagatatgctcttgggatttttggtgatgcctgtgagtatgctcacgatactgtatggatatcgctggcctctgccgtctaaactttgcgctgtgtggatttacttggatgtccttttttcaactgcgagtattatgcatctttgcgccattagtcttgataggtatgtggctatccaaaatcctaagcaccattcccgctttaatagtagaactaaggcttttctgaaaataatagcagtgtggaccatatctgtcggcataagcatgcctatccccgtatttggacttcaagatgactcaaaggtattcaaagaagggtcatgtctgctggccgatgacaatttcgtgcttattggcagcttcgtcagtttcttcattcctttgacaatcatggtgattacctactttcttacgattaaatctttgcaaaagcagctgagctcaggttataacgtctatatcaaggccgacaagcagaagaacggcatcaaggcgaacttcaagatccgccacaacatcgaggacggcggcgtgcagctcgcctaccactaccagcagaacacccccatcggcgacggccccgtgctgctgcccgacaaccactacctgagcgtgcagtccaaactttcgaaagaccccaacgagaagcgcgatcacatggtcctgctggagttcgtgaccgccgccgggatcactctcggcatggacgagctgtacaagggcggtaccggagggagcatggtgagcaagggcgaggagctgttcaccggggtggtgcccatcctggtcgagctggacggcgacgtaaacggccacaagttcagcgtgtccggcgagggtgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacatccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactttaatatgcatgaccaactgaatgagcaaaaggcttgtaaggtactcggcatagtcttctttctgtttgtggtgatgtggtgtcccttctttataacgaatatcatggcagtgatctgcaaggaatcatgcaatgaggatgtgatcggggcacttctgaacgttttcgtgtggatagggtatctgtcaagtgctgtgaacccactggtctataccttgtttaataagacataccgctcagccttttcacggtatattcaatgtcagtataaggaaaacaagaaacctctgcaacttattcttgtgaacactatccctgccctggcttataagtcatcacagttgcagatgggccagaaaaaaaattccaagcaggacgcgaagacaacagacaacgattgtagtatggttgccctcggcaagcagcacagtgaagaagcgagcaaagacaatagtgatggcgtaaacgaaaaagtcagttgtgtattctgctacgagaacgaggtgtaa